SEQUENCE LISTING (11 sequences)

SEQ ID NO: 1 - protein, 472 aa, Mus musculus
MDKLGQIIKLGQLIYEQCEKMKYCRKQCQRLGNRVHGLLQPLQRLQAQGKKNLPDDITAALGRFDEVLKEANQQIEKFSKKSHIWKFVSVGNDKILFHEVNEKLRDVWEELLLLLQVYHWNTVSDVSQPASWQQEDRQDAEEDGNENMKVILMQLQISVEEINKTLKQCSLKPTQEIPQDLQIKEIPKEHLGPPWTKLKTSKMSTIYRGEYHRSPVTIKVFNNPQAESVGIVRFTFNDEIKTMKKFDSPNILRIFGICIDQTVKPPEFSIVMEYCELGTLRELLDREKDLTMSVRSLLVLRAARGLYRLHHSETLHRNISSSSFLVAGGYQVKLAGFELSKTQNSISRTAKSTKAERSSSTIYVSPERLKNPFCLYDIKAEIYSFGIVLWEIATGKIPFEGCDSKKIRELVAEDKKQEPVGQDCPELLREIINECRAHEPSQRPSVDGRSLSGRERILERLSAVEESTDKKV

SEQ ID NO: 2 - protein, 471 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 3 - protein, 8 aa, Artificial Sequence
  synthetic peptide
SIINFEKL

SEQ ID NO: 4 - protein, 16 aa, Artificial Sequence
  synthetic peptide
SQAVHAAHAEINEAGR

SEQ ID NO: 5 - protein, 27 aa, Artificial Sequence
  synthetic peptide
PLLPFYPPDEALEIGLELNSSALPPTE

SEQ ID NO: 6 - protein, 27 aa, Artificial Sequence
  synthetic peptide
VILPQAPSGPSYATYLQPAQAQMLTPP

SEQ ID NO: 7 - protein, 27 aa, Artificial Sequence
  synthetic peptide
DKPLRRNNSYTSYIMAICGMPLDSFRA

SEQ ID NO: 8 - protein, 27 aa, Artificial Sequence
  synthetic peptide
EVIQTSKYYMRDVIAIESAWLLELAPH

SEQ ID NO: 9 - protein, 27 aa, Artificial Sequence
  synthetic peptide
EHIHRAGGLFVADAIQVGFGRIGKHFW

SEQ ID NO: 10 - protein, 27 aa, Artificial Sequence
  synthetic peptide
PSKPSFQEFVDWENVSPELNSTDQPFL

SEQ ID NO: 11 - protein, 27 aa, Artificial Sequence
  synthetic peptide
PSKPSFQEFVDWEKVSPELNSTDQPFL

(19) United States
(12) Patent Application Publication
(10) Pub. No.: US 2021/0121522 A1
(43) Pub. Date: Apr. 29, 2021

(54) MIXED-LINEAGE KINASE DOMAIN-LIKE PROTEIN IN IMMUNOTHERAPEUTIC CANCER CONTROL

(71) Applicants: VIB VZW, Gent (BE); UNIVERSITY GENT, Gent (BE)

(72) Inventors: Xavier Saelens, Ieper (BE); Lien Van Hoecke, Lochristi (BE); Stefaan De Koker, Ressegem (BE); Johan Grooten, Sint-Niklaas (BE)

(21) Appl. No.: 16/645,121

(22) PCT Filed: Sep. 7, 2018
(86) PCT No.: PCT/EP2018/074201

§ 371 Date: Mar. 6, 2020

(30) Foreign Application Priority Data
Sep. 7, 2017 (EP) 17189929.7
May 22, 2018 (GB) 1808377.4

(51) Int. Cl. A61K 38/17; A61K 45/06; C12N 9/12; A61P 35/00

(52) U.S. Cl. CPC A61K 38/1709; A61K 45/06; C12Y 207/99; A61P 35/00; C12N 9/12

(57) ABSTRACT

The invention relates to the field of immuno-oncology. More in particular, it relates to applying the mixed-lineage kinase domain-like protein (MLKL) or variants thereof in immunotherapeutic treatment of cancer. The MLKL or variant thereof is inducing an adaptive immune response to cancer cells leading to treatment of primary tumors and preventing development of secondary tumor or tumor metastasis.

### FIELD OF THE INVENTION

The invention relates to the field of immuno-oncology. More in particular, it relates to applying the mixed-lineage kinase domain-like protein (MLKL) or variants thereof in immunotherapeutic treatment of cancer. The application of MLKL or variant thereof is inducing an adaptive immune response to cancer cells leading to treatment of primary tumors and preventing development of secondary tumor or tumor metastasis.

### BACKGROUND

Worldwide, cancer is a leading cause of death and still until today several malignancies remain incurable or cannot be treated successfully (Torre et al. 2012, CA: a cancer journal for clinicians 65:87-108). Thus, the search for new strategies in anti-tumor therapies is still ongoing. Over the past decade, immunotherapies, which are based on (re)-activating anti-tumor T cells, e.g. by so called check point inhibitors, have substantially increased the success of cancer treatment (Schreiber et al. 2011, Science 331:1565-1570; Hodi & Dranoff 2010, J Cutaneous Pathol 37 Suppl 1:48-53; Hodi et al. 2010, NEJM 363:711-723; Hodi 2010, Asia-Pacific J Clin Oncol 6 Suppl 1:S16-S23). However, until today it remains very challenging to induce a protective or curative anti-tumor T cell response in patients since the majority of patients do not respond to immunotherapy. New insight in the working mechanism of more conventional anti-cancer treatment modalities such as radiotherapy and certain chemotherapeutics (anthracyclines) showed that cancer cells can die in an immunogenic fashion (Zitvogel et al. 2010, Cell 140:798-804; Krysko et al. 2012, Nature Rev Cancer 12:860-875). Immunogenic cell death is a common denominator for diverse cell death pathways that result in the release or exposure of damage-associated molecular patterns (DAMPs) that are normally confined to the intracellular space. These DAMPS are subsequently recognized by Batf3 dependent CD103 DCs that have the capacity to cross-present antigens from the dying cells to T cells and thereby prime effector T cell responses. When DAMP release coincides with the uptake of tumor (neo)-antigens by DCs, potent T cell responses can be elicited against those antigens.

Next to immunogenic death of neoplastic cells that has been documented in response to anthracycline treatment or radio-therapy, necroptosis—a form of regulated necrosis—can also result in an immunogenic response. This was demonstrated by the injection of necroptotic cancer cells into nave mice, which resulted in the maturation of dendritic cells (DCs) and the cross-priming of cytotoxic T-cells (Aaes et al. 2016, Cell Rep 15:274-287). Moreover, a prophylactic injection of necroptotic cancer cells was associated with partial immunity against challenge with live homologous tumor cells in mice (Aaes et al. 2016, Cell Rep 15:274-287; Yatim et al. 2015, Science 350:328-334). The necroptosis-inducer of choice used by Aaes et al. 2016 (Cell Rep 15:274-287) was RIPK3. Vaccines or therapies that can elicit immunogenic cell death might therefore yield the robust T cell response that is required to combat tumors. However, the injection of dying cancer cells in the clinic is impractical and time consuming because it requires excision of tumor cells, the ex vivo induction of immunogenic cell death in the tumor cells and subsequent immunization of the patient with autologous dying tumor cells. Such therapeutic modality will clearly also raise ethical concerns and face harsh regulatory hurdles as reproducible procedures avoiding re-transmission of live cancer cells in a patient will need to be designed.

An anti-tumor T cell response should preferentially be directed against epitopes derived from antigens that are selectively displayed by the tumor cells and not by normal cells. Such so called neo-antigens are the products of tumor-specific mutations and are ideal targets for cancer immunotherapy. Unfortunately, every patient's tumor possesses a unique set of mutations, known as the mutanome, which must first be identified before a personalized therapeutic vaccine can be applied. This is a very time consuming and expensive process that makes the systematic targeting of neo-antigens by vaccine approaches very challenging.

WO01/60991 discloses a series of amino acid and nucleotide sequences of human kinases dubbed as “PKIN”. PKIN-11 corresponds to full-length mixed-lineage kinase domain-like protein (MLKL) as also referred to in WO2010/122135. Both WO01/60991 and WO2010/122135 wrongly refer to MLKL as being an active protein kinase instead of being an inactive pseudokinase (Murphy et al. 2015, Immunity 39:443-453). WO01/60991 does not disclose any functional or other data on PKIN-11 (or any other PKIN).

The data provided in WO2010/122135 are irreconcilably confusing as independently both overexpression of MLKL (Example 3) and inhibition of MLKL by siRNA (FIG. 11) decreases viability of U373-MG, H1299 and MCF7 cells. WO2010/122135 further defines MLKL as an oncogene (thus involved in initiation and progression of tumors). Induction of cell death by lentiviral-driven expression of MLKL or truncated MLKL-variant (truncated to contain basically the full N-terminal four-helical bundle domain) was reported to induce necroptosis in healthy human embryonic kidney-derived 293T (HEK293T) cells (Dondelinger et al. 2014, Cell Rep 7:971-981). HeLa cells stably expressing RIPK3 (receptor-interacting kinase 3) and transfected with plasmid-encoded MLKL showed about 10% of cell death with undimerized MLKL, and about 30% of cell death with dimerized MLKL (MLKL recombinantly modified to comprise an inducible dimerizing fragment). This cell death was dependent on RIPK3 function (Wang et al. 2014, Mol Cell 54:133-146). It is meanwhile widely accepted that both RIPK3 and MLKL are required in order to enable necroptosis to happen (Geserick et al. 2015, Cell Death and Disease 6:e1884; Murphy et al. 2013, Immunity 39:443-453; Sun et al. 2012, Cell 148:213-217; Tanzer et al. 2015, Biochem J 471:255-265) and reactivation of RIPK3 expression (absent or very low in many tumor cells) was suggested as an option for treating metastatic melanoma (Geserick et al. 2015, Cell Death and Disease 6:e1884). Except for the administration of necroptotic tumor cells, the above described experiments were performed in vitro, and thus do not provide any dues on induction of necrosis or induction of immunogenic responses in the more complex in vivo environment. However, even RIPK3 may not be sufficient to elicit tumor immunity; instead NF-KB-induced transcription was reported to be essential for this process (Yatim et al. 2015, Science 350:328-334). Recently, a new function of MLKL was described by Yoon et al. 2017 (Immunity 47:51-65) as a regulator of endosomal trafficking and extracellular vesicle generation.

The core necroptotic pathway involves phosphorylation of receptor interacting protein kinase 3 (RIPK3), which subsequently phosphorylates mixed lineage kinase domain-like protein (MLKL) (Sun et al. 2012, Cell 148:213-227; Zhao et al. 2012, PNAS 109:5322-5327; Murphy et al. 2013, Immunity 39:443-453; Wang et al. 2014, Mol Cell 54:133-146; Dondelinger et al. 2014, Cell Reports 7:971-981; Cai et al. 2014, Nature Cell Biol 16:55-65). Phosphorylated MLKL oligomerizes and subsequently translocates to the plasma-membrane where it inflicts membrane permeabilization and necroptosis (Wang et al. 2014, Mol Cell 54:133-146; Dondelinger et al. 2014, Cell Reports 7:971-981; Cai et al. 2014, Nature Cell Biol 16:55-65; Su et al. 2014, Structure 22:1489-1500; Tanzer et al. 2016, Cell Death Diff 23:1185-1197; Hildebrand et al. 2014, PNAS 111:15072-15077). Strikingly, genetic and epigenetic changes in the pathways that lead to necroptosis have been described for many tumor types. Strongly reduced RIPK3 expression levels, the kinase that phosphorylates and thereby activates MLKL, for example, have been documented in colon carcinoma and are frequent in acute myeloid and chronic lymphocytic leukemia (Moriwaki et al. 2015, Cell Death Dis 6:e1636). Moreover, in pancreatic cancers, reduced MLKL expression is associated with decreased survival (Colbert et al. 2013, Cancer 119:3148-3155; He et al. 2013, Oncotargets Ther 6:1539-1543).

In any case, in situ induction of necroptosis in tumors continues to represent a major challenge, as many tumor types display genetic and epigenetic alterations in the pathways leading to necroptosis. Even more challenging is the in vivo/in situ induction of tumor-specific immune responses, certainly in view of tumor intrinsically/actively/adaptively avoiding or suppressing such responses.

### SUMMARY OF THE INVENTION

In a first aspect, the invention relates to a nucleic acid encoding a mixed-lineage kinase domain-like protein (MLKL) or an isolated MLKL protein for use in (a method of) immunotherapeutic treatment, immunotherapeutic suppression or immunotherapeutic inhibition of a tumor, cancer, or neoplasm in a mammal harboring a tumor, cancer or neoplasm.

Alternatively, the invention relates to a nucleic acid encoding a mixed-lineage kinase domain-like protein (MLKL) or an isolated MLKL protein for use in (a method of) inducing or enhancing necroptotic-like death of tumor, cancer or neoplasm cells in a mammal harboring a tumor, cancer or neoplasm.

Further alternatively, the invention relates to a nucleic acid encoding a mixed-lineage kinase domain-like protein (MLKL) or an isolated MLKL protein for use in (a method of) inducing or enhancing an immune response to tumor, cancer, or neoplasm cells in a mammal harboring a tumor, cancer or neoplasm. In particular, the immune response may be an adaptive immune response or may be a cellular immune response

The above alternatives may be combined in any way, and may further, individually or already combined in any way, be combined with the second aspect of the invention.

In a second aspect, the invention relates to a nucleic acid encoding a mixed-lineage kinase domain-like protein (MLKL) or an isolated MLKL protein for use in (a method of) treating, suppressing or inhibiting secondary tumor, cancer or neoplasm growth, or for use in (a method of) treating, suppressing or inhibiting tumor, cancer or neoplasm metastasis, in a mammal harboring a tumor, cancer or neoplasm.

In any of the above, the tumor, cancer or neoplasm cell may in particularly be deficient in receptor-interacting serine/threonine protein kinase 3 (RIPK3).

In any of the above, the nucleic acid encoding a MLKL or isolated MLKL protein may be combined with a further therapy against the tumor, cancer or neoplasm. Such further therapy may for instance be surgery, radiation, chemotherapy, immune checkpoint or other immune stimulating therapy, neo-antigen or neo-epitope vaccination, cancer vaccine administration, oncolytic virus therapy, antibody therapy, or any other nucleic acid therapy targeting or treating the tumor, cancer or neoplasm.

In any of the above, the nucleic acid encoding a MLKL may be encoding a full-length wild-type MLKL protein, a full-length MLKL protein comprising an amino acid substitution, a fragment of wild-type MLKL protein, or a fragment of a MLKL protein wherein the fragment is comprising an amino acid substitution. Likewise, in any of the above, the isolated MLKL protein may be a full-length wild-type MLKL protein, a full-length MLKL protein comprising an amino acid substitution, a fragment of wild-type MLKL protein, or a fragment of a MLKL protein wherein the fragment is comprising an amino acid substitution.

The invention further relates to nucleic acids encoding a full-length wild-type MLKL protein, a full-length MLKL protein comprising an amino acid substitution, a fragment of wild-type MLKL protein, or a fragment of a MLKL protein wherein the fragment is comprising an amino acid substitution, for use as a medicament.

The invention also relates to isolated full-length wild-type MLKL proteins, isolated full-length MLKL proteins comprising an amino acid substitution, isolated fragments of wild-type MLKL protein, or isolated fragments of a MLKL protein wherein the fragments are comprising an amino acid substitution, for use as a medicament.

Pharmaceutical compositions are also part of the invention and these compositions can comprise an isolated full-length wild-type MLKL protein, an isolated full-length MLKL protein comprising an amino acid substitution, an isolated fragment of wild-type MLKL protein, or an isolated fragment of a MLKL protein wherein the fragment is comprising an amino acid substitution; or can comprise a nucleic acid encoding a full-length wild-type MLKL protein, a full-length MLKL protein comprising an amino acid substitution, a fragment of wild-type MLKL protein, or a fragment of a MLKL protein wherein the fragment is comprising an amino acid substitution; or can comprise a combination of any thereof.

Such pharmaceutical compositions may be for use in (a method of) immunotherapeutic treatment, immunotherapeutic suppression or immunotherapeutic inhibition of a tumor, cancer, or neoplasm in a mammal; for use in (a method of) inducing or enhancing necroptotic-like death of tumor, cancer or neoplasm cells in a mammal; for use in (a method of) inducing or enhancing an immune response to tumor, cancer, or neoplasm cells in a mammal; for use in (a method of) treating, suppressing or inhibiting secondary tumor, cancer or neoplasm growth in a mammal; or for use in (a method of) treating, suppressing or inhibiting tumor, cancer or neoplasm metastasis, in a mammal; wherein the mammal is harboring a tumor, cancer or neoplasm. In these uses/methods, such pharmaceutical compositions may combined with further therapy as described above.

In any of the above, the nucleic acid may be a hypo-inflammatory nucleic acid or a modified nucleic acid.

In any of the above, the nucleic acid may be DNA or RNA. In case of it being DNA, it may be naked DNA, plasmid DNA, DNA included in a viral vector, or complexed DNA (e.g. complexed with lipids or nanomaterials). In case of it being RNA, it may be naked RNA, RNA included in a viral vector, mRNA, or complexed (m)RNA (e.g. complexed with lipids or nanomaterials). Combinations (in any order or timing) of any of these are also envisaged by the current invention. If, in any of the above, the nucleic acid is mRNA, the mRNA may comprise elements such as a 5′ cap and/or a 3′ poly(A)tail and/or a 5′ untranslated region and/or a 3′ untranslated region.

In order to obtain the outlined clinical or therapeutic effects, the nucleic acid encoding a MLKL protein or the isolated MLKL protein for use (in methods) outlined above is administered to the tumor, cancer or neoplasm. Such administration may for instance be by intra-tumor, intra-cancer or intra-neoplasm delivery, or may for instance be remote administration of the nucleic acid (administration remotely from the tumor, cancer or neoplasm), optionally combined with for instance a tumor-, cancer- or neoplasm-targeting moiety.

In any of the above, the nucleic acid encoding a MLKL protein according to the invention may be designed such that expression of MLKL protein in the tumor, cancer or neoplasm is transient, or, in the alternative, inducible.

### DESCRIPTION TO THE FIGURES

FIG. 1. Intra-tumoral MLKL mRNA protects against primary tumor growth In a B16 and CT26 tumor model

500,000 B16-OVA cells (A) or CT26-OVA (B) were s.c. inoculated in the right flank of C57BL/6J mice or Balb/cAnNCrI mice. At day 6 and 10 mice were intra-tumoral injected with saline or 10 μg mRNA encoding luciferase, tBid or MLKL followed by electroporation (two pulses of 20 ms and 120 V/cm). Tumor growth was measured over time. When the tumor became bigger than 2,000 mm³ mice were sacrificed. n=5 representative for three independent experiments. **p<0.01; ***p<0.001; ****p<0.0001 (Log-rank test of Kaplan Meier curves)

FIG. 2. Intra-tumoral MLKL mRNA protects against tumor rechallenge in a B16 and CT26 tumor model

500,000 B16-OVA cells (A) or CT26-OVA cells (B) were s.c. inoculated in the flank of C57BL/6J mice or Balb/cAnNCrI mice. At day 6 and 10 mice were intra-tumoral injected with saline or 10 μg mRNA encoding luciferase, tBid or MLKL followed by electroporation (two pulses of 20 ms and 120 V/cm). The primary tumor was removed at day 12 and two days later a second inoculation of 500,000 B16 or CT26 cells in the left flank of the mice was performed. Tumor growth was measured over time. When the tumor became bigger than 2000 mm³ mice were sacrificed. n=5 representative for three independent experiments. *p<0.0.5; **p<0.01 (Log-rank test of Kaplan Meier curves)

FIG. 3. Intra-tumoral MLKL mRNA protects against metastasis in a B16 and CT26 tumor model

500,000 B16-OVA cells (A) or CT26-OVA cells (B) were s.c. inoculated in the flank of C57BL/6J mice or Balb/cAnNCrI mice. At day 6 and 10 mice were intra-tumoral injected with saline or 10 μg mRNA encoding luciferase, tBid or MLKL followed by electroporation (two pulses of 20 ms and 120 V/cm). The primary tumor was removed at day 12. Two days later, 200,000 B16-F10 melanoma cells or CT26 cells were injected intravenously (i.v.) as a model for metastasis. Mice were sacrificed 12 days after i.v. injection and tumor nodules in the lungs were counted. n=5 representative for two independent experiments. **p<0.01 (Kruskal-Wallis test)

FIG. 4. Intra-tumoral treatment with MLKL mRNA instigates anti-tumor CD8⁺ and CD4⁺ T-cell immunity

500,000 B16 cells were s.c. inoculated in the flank of C57BL/6J mice.
A) Two days prior to treatment, CFSE-labelled OT-I or OT-II cells were adoptively transferred to the inoculated C57BL/6J mice. At day 12, mice were intra-tumoral injected with saline or 10 μg mRNA encoding luciferase, tBid or MLKL followed by electroporation (two pulses of 20 ms and 120 V/cm). Four days after immunization the draining inguinal lymph nodes were isolated and OT-I cell or OT-II cell proliferation was analyzed by flow cytometry. n=5 representative for two independent experiments. **p<0.01; ***p<0.001 (Kruskal-Wallis test)
B) Mice were intra-tumoral injected with saline or 10 μg mRNA encoding luciferase, tBid or MLKL followed by electroporation (two pulses of 20 ms and 120 V/cm) at day 6 and 10. Three days after the second treatment, a mixture of CFSE labeled naïve splenocytes pulsed with control peptide (CFSE^{low}) or OVA peptide (CFSE^{high}) were adoptively transferred to the treated mice. Specific killing was measured two day later by flow cytometry. Data are presented as means of (1−((CFSE^{high}/CFSE^{low})^{immunized mice}/(CFSE^{high}/CFSE^{low})^{mock mice}))×100. n=5 representative for three independent experiments. ***p<0.001 (Kruskal-Wallis test)
C) Mice were intra-tumoral injected with saline or 10 μg mRNA encoding luciferase, tBid or MLKL followed by electroporation (two pulses of 20 ms and 120 V/cm) at day 6 and 10. Three days after the second treatment, spleens were isolated and the number of MHC-class I and class II binding OVA peptide-specific interferon-γ spot-forming splenocytes was determined by enzyme-linked immunosorbent spot (ELISPOT) *p<0.05; ***p<0.001 (Kruskal-Wallis test)

FIG. 5. Intra-tumoral treatment with MLKL mRNA induces T cell responses directed against neo-epitopes in B16 and CT26 tumor model

500,000 B16 cells (A) or CT26 cells (B) were s.c. inoculated in the flank of C57BL/6J mice (A) or Balb/cAnNCrI mice (B). Mice were intra-tumoral injected with saline or 10 μg mRNA encoding luciferase, tBid or MLKL followed by electroporation (two pulses of 20 ms and 120 V/cm) at day 6 and 10. Three days after the second treatment, spleens were isolated and the number of neo-epitope-specific intereferon-γ spot-forming splenocytes was determined by enzyme-linked immunosorbent spot (ELISPOT). For the B16 model (A) we used the CD4 T cell B16M30 epitope and for the CT26 model (B) the CD8 T cell CT26-M26 epitope and the CD4 T cell CT26-M20, CT26-M03, CT26-M37 and CT26-M27 epitopes. These epitopes have been described in Kreiter et al 2015 (Nature 520:692-696) as mutant neo-epitopes that can be used to induce anti-tumor immunity. *p<0.05; **p<0.01 (Kruskal-Wallis test)

FIG. 6. Lymphocyte infiltration in the tumor-draining lymph node after treatment with MLKL mRNA

500,000 B16-OVA cells were s.c. inoculated in the flank of C57BL/6J mice. At day 6 and 10, mice were intra-tumoral injected with saline or 10 μg mRNA encoding luciferase, tBid or MLKL followed by electroporation (two pulses of 20 ms and 120 V/cm). Two days after the second treatment the tumor draining lymph node was dissected and the influx of monocyte derived dendritic cells (moDCs), conventional dendritic cells (cDC) type 1 and type 2 was analyzed via flow cytometry.

FIG. 7. CD8α DCs, CD8⁺ and CD4⁺ T cells are important in the protection mechanism of intra-tumoral treatment with MLKL mRNA
A) 500,000 B16-OVA cells were s.c. inoculated in the flank of CCR7^{ko} mice. Two days prior to treatment, CFSE-labelled OT-I cells were adoptively transferred to the inoculated CCR7^{ko} mice. Mice were intra-tumoral injected with saline or 10 μg mRNA encoding luciferase, tBid or MLKL followed by electroporation (two pulses of 20 ms and 120 V/cm) at day 12. Four days after treatment the draining inguinal lymph nodes were isolated and OT-I cell proliferation was analyzed by flow cytometry. n=5
B) 500,000 B16-OVA cells were s.c. inoculated in the flank of batf3^{ko} mice. Mice were intra-tumoral injected with saline or 10 μg mRNA encoding luciferase, tBid or MLKL followed by electroporation (two pulses of 20 ms and 120 V/cm) at day 6 and 10. Three days after the second treatment, a mixture of CFSE labeled splenocytes pulsed with control (CFSE^{low}) or OVA peptide (CFSE^{high}) were adoptively transferred to the immunized mice. Specific killing was measured two day later by flow cytometry. Data are presented as means of (1−((CFSE^{high}/CFSE^{low})^{immunized mice}/(CFSE^{high}/CFSE^{low})^{mock mice}))×100; n=5
C) 500,000 B16-OVA cells were s.c. inoculated in the flank of ifnar^{ko} mice. Mice were intra-tumoral injected with saline or 10 μg mRNA encoding luciferase, tBid or MLKL followed by electroporation (two pulses of 20 ms and 120 V/cm) at day 6 and 10. Three days after the second treatment, a mixture of CFSE labeled splenocytes pulsed with control (CFSE^{low}) or OVA peptide (CFSE^{high}) were adoptively transferred to the immunized mice. Specific killing was measured two day later by flow cytometry. Data are presented as means of (1−((CFSE^{high}/CFSE^{low})^{immunized mice}/(CFSE^{high}/CFSE^{low})^{mock mice}))×100; n=5
D) 500,000 B16 cells were s.c. inoculated in the flank of C57BL/6J mice. At day 6 and 10 mice were intra-tumoral injected with saline or 10 μg mRNA encoding luciferase, tBid or MLKL followed by electroporation (two pulses of 20 ms and 120 V/cm). Tumor growth was measured over time. When the tumor became bigger than 2,000 mm³ mice were sacrificed. At day 5 and 10 after tumor inoculation CD8⁺ T cells were depleted via i.p. injection of 200 μg anti-mouse CD8α antibody.
E) Kaplan-Meier plot showing the impact of CD4⁺ T cell depletion and CD8⁺ T cell depletion on the anti-tumor effect of intratumor MLKL-mRNA treatment.

FIG. 8. characterization of designed mRNAs
A) Designed hypo-inflammatory mRNA's: the 5′ and 3′ untranslated region (UTR) of human B-globulin (HBB) was added upstream and downstream of the coding sequences to increase the stability of the mRNAs. A poly-A (60) tail was added 3′ of the constructs. Next an O-methylated 5′ m7-cap was ligated postranscriptionally to the in vitro produced mRNAs.
B) mRNA coding for MLKL and mRNA coding for MLKL where RNAse A was added to loaded on a 1% agarose gel.
C) In vitro transfection and translation efficiency: Cy5-labeled mRNA coding for GFP was transfected into B16-OVA cells. At different time points after transfection Cy5 and GFP fluorescence were analyzed by flow cytometry. Cy5 positivity is a measurement of transfection efficiency while GFP positivity is a measurement of translation efficiency. Gating strategy: cells were selected based on forward scatter (FSC) and side scatter (SSC). Next cy5 and GFP positivity was analyzed at different time points after transfection.
D) In vivo translation efficiency: 500,000 B16-OVA cells were s.c. inoculated in the flank of C57BL/6J mice. At day 6 mice were intra-tumoral injected with 10 μg luciferase encoding mRNA followed by electroporation (left mice) or not (right mice). D-luciferine was injected intraperitoneally at different time points after mRNA injection and the luciferase activity was measured by whole body imaging.

FIG. 9. MLKL coding mRNA induces necroptotic like cell death while tBid coding mRNA induces apoptotic like cell death In vitro and in vivo.
(A) B16-OVA cells were transfected in vitro with no mRNA, luciferase encoding mRNA, tBid encoding mRNA or MLKL encoding mRNA. At different time points cells were collected and stained with SYTOX blue for death cells and annexin-V for phosphatidylserine exposure at the membrane. Percentages of annexin⁺ SYTOX⁺ cells (left) and annexin⁻ SYTOX⁺ cells (right) of the total single cell population are shown.
(B) B16-OVA cells were transfected in vitro with saline or mRNA encoding luciferase, tBid or MLKL. The cell death progression was measured by sytox green fluorescence and caspase activity was measured by DEVD-AMC cleavage over time. Caspase activity was in some conditions blocked with the Pan-Caspase inhibitor zVAD-fmk.
(C) B16-OVA cells were transfected in vitro with saline or mRNA encoding luciferase, tBid or MLKL. The cell death progression was visualized via time-lapse microscopy.
(D) 500 000 B16-OVA tumor cells were subcutaneously (s.c.) inoculated in the flank of C57BL/6J mice. 7 days after inoculation the tumor was injected with 10 μg mRNA encoding luciferase, tBid or MLKL followed by electroporation (two pulses of 20 ms and 120 V/cm). 24 h after electroporation, tumors were isolated and stained with SYTOX blue for death cells and annexin-V for phosphatidylserine exposure at the membrane. Graphs show percentages of sytox⁺ cells and representative flow cytometry plots.

FIG. 10. gating strategy annexin-V and Sytox blue staining

First single cells were selected based on the FSC and SSC. Next annexin-V and sytox blue positivity was analyzed.

FIG. 11. gating strategy OT-I or OT-II proliferation

First single cells were selected based on the FSC and SSC. Next T-cells were gated as CD3⁺ CD19⁻ cells. In this T-cell population CD8⁺ T cells (in OT-I proliferation assay) or CD4⁺ T cells (in OT-II proliferation assay) were selected. The FITC profile of the OVA⁺ CD8⁺ T or CD4⁺ T cells was analyzed.

FIG. 12. gating strategy killing assay

First single cells were selected based on the FSC and SSC. Next the CFSE profile was analyzed in the CFSE⁺ population.

FIG. 13. Gating strategy BMDC

First alive single cells were selected based on the FSC, SSC and live/death staining. Next macrophages and BMDC were gated based on the CD11c⁺ and MHCII⁺ expression. In this gate macrophages were further identified as CSF1R⁺ cells while BMDC were identified as CD26⁺ cells.

FIG. 14. Gating strategy dendritic cells

First alive single cells were selected based on the FSC, SSC and live/death staining. Next T-cells and B-cells were gated out based on the CD3 and CD19 expression respectively. moDC were identified as CD64⁺MHCII⁺ cells, cDC1 were identified as MHCII⁺CD11c⁺XCR1⁺ cells and cDC1 were identified as MHCII⁺CD11c⁺CD172α⁺ cells.

FIG. 15. MLKL encoding mRNA Induces cell death in vitro and In vivo. In vitro cell death characterization. B16-OVA cells were transfected with PBS or with Fluc- (luciferase), tBid- or MLKL-mRNA. (A) Western blot analysis of the expression of MLKL, caspase-3 and cleaved caspase-3 in cell lysates prepared 24 hours after mRNA transfection. Tubulin served as a loading control. (B) All B16 cells were transfected with a plasmid with the coding sequence of luciferase under control of the NF-κb promoter and a plasmid expressing β-galactosidase for normalization purpose. Twenty four hours later, the cells were transfected with PBS, GFP, tBid or MLKL-mRNA or, as a positive control for NF-κb activation, with TRAF6 expressing plasmid or stimulated with TNF. The normalized luciferase activity in the lysates was determined at different time points after mRNA transfection. (C) B16 cells were transfected with a GFP expression plasmid. Twenty four hours later, the cells were transfected with PBS, Fluc-, tBid- or MLKL-mRNA and the cells were treated or not with actinomycin D as indicated. Twenty four hours after mRNA transfection, cell death and GFP expression were quantified using flow cytometry.

FIG. 16. Intratumor MLKL-mRNA protects better than doxorubicine treatment against primary tumor growth in a B16 tumor model. (A) Schematic representation of the experiment. B16 cells were inoculated s.c. in the right flank of C57BL/6J (n=8 per group). On day 6 and 10, intra tumor injection of PBS, Fluc mRNA, tBid mRNA or MLKL mRNA followed by electroporation was performed. Doxorubicin (dox, 3 mg/kg per injection) was administered i.p. or intra tumorally (i.t.) every second day starting on day 6. One group of mice received 3 intra tumor injections of dox on day 6, 8 and 10. (B) Tumor growth progression overtime depicted for the individual mice in each group. The animals were euthanized when the tumor had reached a size of 1,000 mm³. (C) Survival curves and (D) body weight changes of the treated mice. **p<0.01, ***p<0.001, ****p<0.0001, ns non-significant determined by Log-rank test of the Kaplan Meier survival curves and by one-way ANOVA for the body weight graphs. (E) Hematologic analyses of the number of lymphocytes in blood collected on days 11, 18 and 25 from the treated mice. Each bar represents the average of 8 mice. The Y axis depicts the number of lymphocytes per μl of blood.

FIG. 17. Intratumor MLKL-mRNA treatment protects against tumor rechallenge and reduces growth of a pre-existing untreated tumor. B16-cells were s.c. inoculated in the right flank of C57BL/6J mice. Three days later B16-cells were s.c. inoculated in the left flank. On day 6 and 10 the tumors on the right flank were injected with saline or 10 μg mRNA encoding Fluc, tBid or MLKL followed by electroporation. The growth of the tumor that had been inoculated in the left flank was measured over time. Mice were euthanized when the tumor of the right flank reached 1000 mm³ in size. The experiment was performed once with 5 mice per group in the PBS and luciferase mRNA set up, and 8 mice per group in the tBid- and MLKL-mRNA treatment groups.

FIG. 18. Combined MLKL-mRNA treatment with anti-PD1 inhibition improves the anti-tumor outcome. (A) Schematic representation of the experiment. B16-cells were s.c. inoculated in the right flank of C57BL/6J mice on day 0. Three days later B16-cells were s.c. inoculated in the left flank. On day 6 and 10 the tumor on the right flank was injected with saline or 10 μg mRNA encoding Fluc or MLKL followed by electroporation. Starting from day 6, 200 μg anti PD-1 or isotype control antibody was administered every three days i.p., for 3 weeks or until the ethical endpoint was reached. The growth of the tumor in the right (B) and left flank (C) was monitored, and mice were euthanized when the tumor in the right flank had reached a size of 1000 mm³. *p<0.0.5 (Log-rank test of Kaplan Meier curves). The experiment was performed once with 8 mice per group.

FIG. 19. Lymphocyte infiltration and T cell activation after MLKL-mRNA treatment depends on Batf3 DCs and type I IFN signaling. C57BL/6J or the indicated knockout mice (n=5 per group) were inoculated with B16-OVA cells and treated once (A+B) or twice (C+D+E) with mRNA encoding Fluc or MLKL (A) One day after the first treatment, the tumor was dissected and the influx of conventional type 1 (cDC1) and type 2 DCs (cDC2) was analyzed by flow cytometry. Results are shown as dot plots. **p<0.01 (Mann-Whitney U test). (B) Influx of cDC1 and cDC2 cells in the tumor draining lymph node on day two after the first treatment analyzed by flow cytometry.

FIG. 20. Intratumor MLKL-mRNA treatment protects against primary tumor growth of human RL cells in mice with a humanized immune system. (A) Human melanoma cell lines (501 Mel, BLM, SK-Mel28), human early passage cultures (M010817 and M000921) and human B lymphoma cells (RL cells) were transfected with PBS or with mRNA encoding Fluc or human MLKL. Twenty four hours after transfection cells were collected and analyzed by flow cytometry. The graph shows the percentages of sytox⁺ cells (left) and flow cytometry plots of transfected RL cells (right). (B) Newborn NSG mice (1-2 days of age) were sublethally irradiated and subsequently received 1.10⁵ CD34⁺ human stem cells isolated from HLA-A2 positive cord blood by injection in the liver. Thirteen weeks after stem cell transfer, 2,5×10⁶ human RL follicular lymphoma cells were inoculated s.c. into the mice. On days six and ten (treatment 1 and 2, respectively) the tumors were injected with saline or 10 μg mRNA encoding Fluc or human MLKL followed by electroporation. Starting eight days after tumor inoculation and during the treatments, 30 μg Flt3 ligand was given daily. Tumor growth was measured over time. The animals were culled when the tumor had reached a size of 100 mm². ****p<0.001 (Log-rank test of Kaplan Meier curves).

FIG. 21. Intratumor MLKL-mRNA protects against experimental lung colonization in a B16 and CT26 tumor model. (A) Schematic representation of the experiment. B16-OVA (B) were s.c. inoculated in the flank of C57BL/6J or BALB/cAnNCrI mice, respectively. After intratumor mRNA treatment 1 and 2 and primary tumor removal, the animals received an intravenous injection of B16-F10 melanoma cells (B). Mice were sacrificed 12 days or 22 days after i.v. injection and the number of tumor nodules in the lungs were counted. Results are shown as dot plots. Results shown in (B) are representative for three independent experiments for the day 26 samples, each with 8 mice per group, and from one experiment for the day 36 sampling with 8 mice per group. *p<0.0.5; ***p<0.001; ****p<0.0001; ns non-significant (Kruskal-Wallis test with Dunn's post hoc multiple comparison test).

FIG. 22. Intratumor delivery of MLKL-pDNA protects against primary tumor growth in a B16 tumor model. (A) Schematic representation of the experiment. B16-OVA cells were s.c. inoculated in the right flank of C57BL/6J mice (n=5 per group). On day 6 and 10 (treatment 1 and 2, respectively) the tumors were injected with saline or 100 μg pCAXL or pCAXL-MLKL followed by electroporation. Tumor growth was measured over time. The animals were euthanized when the tumor had reached a size of 1,000 mm³. The upper graph panels in (B) depict tumor growth curves of individual mice and the lower graph depicts percentage survival. ns non-significant; **p<0.01 (Log-rank test of Kaplan Meier curves). The results shown are from 1 experiment that has not yet been repeated.

FIG. 23. Transfection of MLKL encoding mRNA in B16 cells does not induce phosphorylation of the MLKL protein. One million B16 cells were transfected with PBS or with 1 μg of mRNA encoding luciferase, tBid or MLKL. Twenty four hours after transfection, MLKL and phosphorylated MLKL expression were analyzed in the cell lysates using western blotting. As a positive control for phosphorylation of MLKL, L929sAhFas cells were stimulated with TNF during 8 hours and cell lysates were analyzed by western blotting using anti-MLKL (Millipore, MABC604) and anti-phospho-MLKL antibodies (Abcam; an196436).

FIG. 24. Transfection of mRNA encoding a constitutively active mutant of MLKL results in increased cell death. One million B16 cells were transfected with PBS or with 1 μg of mRNA encoding luciferase, tBid, MLKL or MLKLS345D (caMLKL). Twenty four hours after transfection, cell death was monitored by flow cytometry based on sytox blue uptake. Data points represent the percentage of sytox positive cells in the total population of cells. Horizontal lines represent the mean and standard deviation (SD). The insets above the graph are representative flow cytometry plots with forward scatter (FSC) scaled linearly in the X axis and the sytox blue fluorescence scaled logarithmically in the Y axis.

FIG. 25. Effect of intratumor delivery of mRNA encoding different full-length and truncated variants of MLKL (A) Schematic representation of the experiment. B16-OVA cells were s.c. inoculated in the right flank of C57BL/6J mice (n=5 per group for PBS and luciferase; n=8 for MLKL, constitutively active MLKL (caMLKL), non-phosphorylatable MLKL (iaMLKL), MLKL fragment (1-180) and MLKL fragment (180-464). On day 6 and 10 (treatment 1 and 2, respectively) the tumors were injected with saline or 10 μg mRNA encoding Fluc, MLKL, caMLKL, iaMLKL, MLKL (1-180), MLKL (180-464) followed by electroporation. Tumor growth was measured over time. The animals were euthanized when the tumor had reached a size of 1,000 mm³. The upper 7 panels in (B) depict tumor growth curves of individual mice and the lower graph depicts percentage survival. ns non-significant,**p<0.01, ***p<0.001, ****p<0.0001 (Log-rank test of Kaplan Meier curves). The results shown are from 1 experiment.

FIG. 26. Intratumor delivery of MLKL-mRNA provides better protection than RIPK3-mRNA. (A) Schematic representation of the experiment. B16-OVA cells were s.c. inoculated in the right flank of C57BL/6J mice (n=5 per group for PBS and luciferase; n=8 for RIPK3 and MLKL). On day 6 and 10 (treatment 1 and 2, respectively) the tumors were injected with saline or 10 μg mRNA encoding Fluc, RIPK3 or MLKL followed by electroporation. Tumor growth was measured over time. The animals were euthanized when the tumor had reached a size of 1,000 mm³. The upper 4 panels in (B) depict tumor growth curves of individual mice and the lower graph depicts percentage survival. ***p<0.001 (Log-rank test of Kaplan Meier curves). The results shown are from 1 experiment.

### DETAILED DESCRIPTION TO THE INVENTION

Many factors and processes are decisive over whether or not an initial single tumor cell will be able to create, and support, its ecosystem and, therewith, growth. In an attempt to create a simplifying overview, Blank et al. 2016 (Science 352: 658-660) designed a visually appealing “cancer immunogram” in which currently known factors and processes influencing tumor growth/survival are grouped in seven classes of parameters. For each individual patient/tumor, the status of the seven classes of parameters can be plotted, the resulting plot giving insight in treatment options. On the other hand, such immunogram illustrates the complexity of cancer and cries for providing ever more potential therapies from which the most promising can be picked for treatment of an individual cancer in an individual patient. Although immuno-oncology already provided remarkable successes in the form of new cancer therapies, it is a field still in full expansion and its full potential in all likelihood is far from fulfilled. In work leading to the current invention, delivery of mRNA or plasmid DNA encoding the mixed-lineage kinase domain-like protein (MLKL), a fragment thereof, or a variant thereof, was demonstrated to be a promising novel immunotherapeutic anti-cancer treatment. This treatment strongly stalled the growth of primary tumors and protected against distal and metastatic tumors. Importantly, MLKL-mRNA treatment of established tumors elicited a strong CD4⁺ and CD8⁺ T cell response directed against multiple tumor specific neo-antigen epitopes. This strategy to induce a tumor (neo-)antigen-specific T cell response requires no prior knowledge of the nature of these tumor (neo-)antigens and therewith holds promise to be a generic antitumor therapeutic/immunotherapeutic approach. Mechanistically, intra-tumoral MLKL nucleic acid/mRNA/DNA treatment was rapidly followed by a strong influx of dendritic cells (DCs: cDC1s and cDC2s) into the draining lymph nodes. Anti-tumor immunity depended on cross presenting DCs as well as on CD4⁺ and CD8⁺ T cells.

Based hereon, the invention is defined in the following aspects and embodiments.

In a first aspect, the invention relates to a nucleic acid or a (pharmaceutical) composition comprising the nucleic acid for use in (a method of) immunotherapeutic treatment, immunotherapeutic suppression or immunotherapeutic inhibition of a tumor, cancer, or neoplasm in a mammal harboring a tumor, cancer or neoplasm, wherein the nucleic acid is encoding a mixed-lineage kinase domain-like protein (MLKL). Furthermore, the invention relates to a MLKL protein or a (pharmaceutical) composition comprising the MLKL protein for use in (a method of) immunotherapeutic treatment or immunotherapeutic suppression of a tumor, cancer, or neoplasm in a mammal harboring a tumor, cancer or neoplasm.

Alternatively, the invention relates to a nucleic acid or a (pharmaceutical) composition comprising the nucleic acid for use in (a method of) inducing or enhancing necroptotic-like death of tumor, cancer or neoplasm cells in a mammal harboring a tumor, cancer or neoplasm, wherein the nucleic acid is encoding a mixed-lineage kinase domain-like protein (MLKL). Furthermore, the invention relates to a MLKL protein or a (pharmaceutical) composition comprising the MLKL protein for use in (a method of) inducing or enhancing necroptotic-like death of a tumor, cancer, or neoplasm in a mammal harboring a tumor, cancer or neoplasm. Necroptotic-like death may be fully absent or negligible in the tumor, cancer or neoplasm cells prior to administration of the nucleic acid (composition) encoding a MLKL protein or prior to administration of a MLKL protein (composition), and the administration of MLKL-encoding nucleic acid (composition) or MLKL protein (composition) is inducing the process. Alternatively, necroptotic-like death may already be occurring to some extent in the tumor, cancer or neoplasm cells prior to administration of the nucleic acid (composition) encoding a MLKL protein or prior to administration of a MLKL protein (composition) and the administration of MLKL-encoding nucleic acid (composition) or MLKL protein (composition) is enhancing the process.

In particular, the necroptotic-like tumor, cancer or neoplasm cell death is capable of eliciting an immune response, in particular a tumor-, cancer- or neoplasm-specific immune response.

Further alternatively, the invention relates to a nucleic acid or a (pharmaceutical) composition comprising the nucleic acid for use in (a method of) inducing or enhancing an immune response to a tumor, cancer, or neoplasm cells in a mammal harboring a tumor, cancer or neoplasm, wherein the nucleic acid is encoding a mixed-lineage kinase domain-like protein (MLKL). Furthermore, the invention relates to a MLKL protein or a (pharmaceutical) composition comprising the MLKL protein for use in (a method of) inducing or enhancing an immune response to a tumor, cancer, or neoplasm in a mammal harboring a tumor, cancer or neoplasm. An adaptive immune response may be fully absent or negligible in the mammal harboring the tumor, cancer or neoplasm cells prior to administration of the nucleic acid (composition) encoding a MLKL protein or prior to administration of a MLKL protein (composition), and the administration of MLKL-encoding nucleic acid (composition) or MLKL protein (composition) is inducing the process. Alternatively, an adaptive immune response to may already be occurring to some extent in the mammal harboring a tumor, cancer or neoplasm cells prior to administration of the nucleic acid (composition) encoding a MLKL protein or prior to administration of a MLKL protein (composition) and the administration of MLKL-encoding nucleic acid (composition) or MLKL protein (composition) is enhancing the process.

The above alternatives may be combined in any way, and may further be combined with the second aspect of the invention.

In a second aspect, the invention relates to a nucleic acid or a (pharmaceutical) composition comprising the nucleic acid for use in (a method of) treating, suppressing or inhibiting secondary tumor, cancer or neoplasm growth or for use in treating, suppressing or inhibiting tumor, cancer or neoplasm metastasis, in a mammal harboring a tumor, cancer or neoplasm, wherein the nucleic acid is encoding a mixed-lineage kinase domain-like protein (MLKL). Furthermore, the invention relates to a MLKL protein or a (pharmaceutical) composition comprising the MLKL protein for use in (a method of) treating, suppressing or inhibiting secondary tumor, cancer or neoplasm growth, or for use in treating, suppressing or inhibiting tumor, cancer or neoplasm metastasis in a mammal harboring a tumor, cancer or neoplasm.

In any of the above aspects and embodiments, the “nucleic acid encoding a MLKL protein” may be encoding a full-length wild-type MLKL protein, a full-length MLKL protein comprising an amino acid substitution (variant or mutant MLKL protein), a fragment of wild-type MLKL protein (MLKL protein fragment), or a fragment of a MLKL protein wherein the fragment is comprising an amino acid substitution (relative to the wild-type MLKL protein fragment; variant or mutant MLKL protein fragment). Nucleic acid in this context is not meant to be a single copy of a nucleic acid molecule but instead is meant to be a population of identical nucleic acid molecules (homogenous population in as far as isolation and purification technologies allow). Likewise, in any of the above instances, the “isolated MLKL protein” may be a full-length wild-type MLKL protein, a full-length MLKL protein comprising an amino acid substitution (variant or mutant MLKL protein), a fragment of wild-type MLKL protein (MLKL protein fragment), or a fragment of a MLKL protein wherein the fragment is comprising an amino acid substitution (relative to the wild-type MLKL protein fragment; variant or mutant MLKL protein fragment). Isolated protein in this context is not meant to be a single molecule of a protein but instead is meant to be a population of identical protein molecules (homogenous population in as far as isolation and purification technologies allow).

Further aspects of the invention relate to (i) nucleic acids encoding a full-length wild-type MLKL protein, a full-length MLKL protein comprising an amino acid substitution, a fragment of wild-type MLKL protein, or a fragment of a MLKL protein wherein the fragment is comprising an amino acid substitution, all for use as a medicament; and (ii) to isolated full-length wild-type MLKL proteins, isolated full-length MLKL proteins comprising an amino acid substitution, isolated fragments of wild-type MLKL protein, or isolated fragments of a MLKL protein wherein the fragments are comprising an amino acid substitution, all for use as a medicament.

In the above instances wherein an immune response is induced, this may be an adaptive immune response, in particular this may be a cellular immune response.

In any of the above aspects and embodiments, the MLKL protein or MLKL protein encoded by the nucleic acid may comprise a variation such as an amino acid mutation rendering it into a “phosphomimetic” MLKL variant, or rendering it into a non-phosphorylatable MLKL variant. The MLKL protein or MLKL protein encoded by the nucleic acid may be a truncated version of wild-type full-length MLKL protein (truncated MLKL protein, or MLKL protein fragment) or may be a truncated version of a MLKL protein (fragment of MLKL protein) wherein the truncated MLKL/MLKL protein fragment is comprising a variation or mutation (fragment of a variant MLKL protein, or truncated variant MLKL protein); in particular, a truncated MLKL protein may for instance be only an N-terminal part (known as four α-helical domain or 4HD, see further) or may for instance be only the C-terminal pseudokinase domain of MLKL; any MLKL fragment or truncated MLKL protein may optionally further comprise one or more amino acid variations or mutations (relative to wild-type MLKL protein). Further in particular the variant or fragment of MLKL is a membrane-permeabilizing variant of MLKL or a membrane-permeabilizing fragment of MLKL.

In any of the above aspects and embodiments, the MLKL protein, variant MLKL protein, fragment of MLKL protein or fragment of a variant MLKL protein in particular is an isolated protein, such as isolated and/or purified after recombinant production in a suitable host.

A mammal “harboring a tumor, cancer or neoplasm” is meant to be a mammal suspected to have, to carry, or to suffer from a tumor, cancer or neoplasm present at any place or organ in the body of the mammal; it may alternatively refer to a mammal actually diagnosed to have, to carry, or to suffer from a tumor, cancer or neoplasm present at any place or organ in the body of the mammal. The diagnosis can be performed by means of any available technology or methodology.

The uses and methods described above in general comprise the administration of the MLKL protein, MLKL variant protein or MLKL fragment protein or nucleic acid encoding MLKL, MLKL variant or MLKL fragment (such as, but not limited to, a membrane-permeabilizing variant of MLKL or a membrane-permeabilizing fragment of MLKL) to the mammal in need thereof, i.e., harboring a tumor, cancer or neoplasm in need of treatment. The administration of the MLKL protein, MLKL variant protein or MLKL fragment protein or nucleic acid encoding MLKL, MLKL variant or MLKL fragment (such as, but not limited to, a membrane-permeabilizing variant of MLKL or a membrane-permeabilizing fragment of MLKL) is leading to the described clinical and/or therapeutic response(s); in general an effective amount of MLKL protein, MLKL variant protein or MLKL fragment protein or nucleic acid encoding MLKL, MLKL variant or MLKL fragment (such as, but not limited to, a membrane-permeabilizing variant of MLKL or a membrane-permeabilizing fragment of MLKL) is administered to the mammal in need thereof in order to obtain the described clinical and/or therapeutic response(s). The effective amount of MLKL protein, MLKL variant protein or MLKL fragment protein or nucleic acid encoding MLKL, MLKL variant or MLKL fragment (such as, but not limited to, a membrane-permeabilizing variant of MLKL or a membrane-permeabilizing fragment of MLKL) will depend on many factors such as route of administration and tumor mass and will need to be determined on a case-by-case basis by the physician.

Terminology as used in describing the aspects of the invention is described in the following sections.

Mixed-Lineage Kinase-Domain Like Protein (MLKL) and Variants

The full-length human MLKL protein is a 471-amino acid defined by Genbank accession number NP 689862 (full-length murine MLKL: Genbank accession number NP_001297542.1). A second human MLKL isoform having 263 amino acids is defined by Genbank accession number NP 001135969 and is identical to full-length MLKL in the N-terminal amino acids 1-178 and C-terminal amino acids 414-471 but is lacking the mid-region of full-length MLKL. Phosphorylated MLKL (phosphorylation by RIPK3, in the necrosome) is translocated to the plasma membrane, a process required to achieve necroptotic membrane rupture (e.g. Murphy et al. 2013, Immunity 39:443-453). The S2D mutant of MLKL (comprising the S345D and S347D mutations in murine MLKL, highlighted in the below sequence alignment; corresponding amino acids in human MLKL being S358 and S360, see below sequence alignment) is a phosphomimetic, constitutively active MLKL variant able to induce necroptosis in the absence of RIPK3 (e.g. Murphy et al. 2013, Immunity 39:443-453). Human MLKL with the S358D+S360D mutations can thus be considered as an active (phosphomimetic) variant. Human MLKL with the T357E+S358D mutations was disclosed to be an alternative active phosphomimetic variant (Xia et al. 2016, Cell Res 26:517-528). Both threonine 357 (T357) and serine 358 (S358) of human MLKL were reported to be phosphorylated by RIPK3 (Sun et al. 2012, Cell 148:213-227). Phosphorylation of a single of these Ser or Thr residues, and thus mutation of a single of these Ser or Thr residues, may be sufficient for converting wild-type MLKL into “activated” MLKL. Versions of MLKL comprising one or more amino acid substitutions relative to the wild-type MLKL are referred to herein as variants of MLKL or point mutants of MLKL. Fragments or truncated forms of MLKL may likewise comprise such amino acid substitutions.

| | | | |
|---|---|---|---|
| murine | 1 | MDKLGQIIKLGQLIYEQCEKMKYCRKQCQRLGNRVHGLLQPLQRLQAQGKKNLPDD-ITA | 59 |
| human | 1 | MENLKHIITLGQVIHKRCEEMKYCKKQCRRLGHRVLGLIKPLEMLQDQGKRSVPSEKLTT | 60 |
| murine | 60 | ALGRFDEVLKEANQQIEKFSKKSHIWKFVSVGNDKILFHEVNEKLRDVWEELLLLLQVYH | 119 |
| human | 61 | AMNRFKAALEEANGEIEKFSNRSNICRFLTASQDKILFKDVNRKLSDVWKELSLLLQVEQ | 120 |
| murine | 120 | WNTVSDVSQPASWQQEDRQDAEED---------GNENMKVILMQLQISVEEINKTLKQ-C | 169 |
| human | 121 | RMPVSPISQGASWAQEDQQDADEDRRAFQMLRRDNEKIEASLRRLEINMKEIKETLRQYL | 180 |
| murine | 170 | SLKPTQEIPQDLQIKEIPKEHL-GPPWTKLKTSKMSTIYRGEYHRSPVTIKVFNNPQAES | 228 |
| human | 181 | PPKCMQEIPQE-QIKEIKKEQLSGSPWILLRENEVSTLYKGEYHRAPVAIKVFKKLQAGS | 239 |
| murine | 229 | VGIVRFTFNDEIKTMKKFDSPNILRIFGICIDQTVKPPEFSIVMEYCELGTLRELLDREK | 288 |
| human | 240 | IAIVRQTFNKEIKTMKKFESPNILRIFGICIDETVTPPQFSIVMEYCELGTLRELLDREK | 299 |
| murine | 289 | 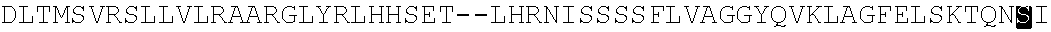 | 346 |
| human | 300 | DLTLGKRMVLVLGAARGLYRLHHSEAPELHGKIRSSNFLVTQGYQVKLAGFELRKTQTSM | 359 |
| murine | 347 | 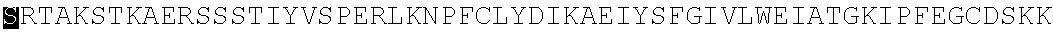 | 406 |
| human | 360 | SLGTTREKTDRVKSTAYLSPQELEDVFYQYDVKSEIYSFGIVLWEIATGDIPFQGCNSEK | 419 |
| murine | 407 | IRELVAEDKKQEPVGQDCPELLREIINECRAHEPSQRPSVDGRSLSGRERILERLSAVEE | 466 |
| human | 420 | IRKLVAVKRQQEPLGEDCPSELREIIDECRAHDPSVRPSVD--------EILKKLSTFSK | 471 |
| murine | 467 | STDKKV 472 [SEQ ID NO: 1] | |
| human | | ------ [SEQ ID NO: 2] | |

Using liposomes, cation-channel formation by MLKL was demonstrated (Xia et al. 2016, Cell Res 26:517-528). MLKL forms tetramers, depending on disulfide bond formation (C169S and C275S mutations in murine MLKL abolishing tetramer formation), and further assembles in octamers. Octamer formation, however, does not require disulfide bond formation as C169S/C275s mutant murine MLKL is capable or forming octamers and of inducing necroptosis. An artificial disulfide bond between C86-residues in human MLKL can be detected but is not functionally relevant as the C86S mutation does not prevent octamer formation and induction of necroptosis. Variants of human MLKL shown to induce octamer formation and/or necroptosis include: T122A; T122S; T122C; T122C+C18S+C24S+C28S; E76A+K77A; W85A+K89A; N92A+D93A+K94A; E102A+K103A (Huang et al. 2017, Mol Cell Biol 37:e00497; Xia et al. 2016, Cell Res 26:517-528). Inactive variants of human MLKL include S79A+K81A and R105A+D106A and apparently MLKL or MLKL fragments comprising a C-terminal extension (such as the FLAG epitope) (Huang et al. 2017, Mol Cell Biol 37:e00497; Hildebrand et al. 2014, Proc Nat Acad Sci USA 111:15072-15077).

Further variants of MLKL include variants truncated to comprise the N-terminal four α-helical domains (4HD) shown both to be able to induce necroptosis (Dondelinger et al. 2014, Cell Rep 7:971-981) as well as disruption of liposome membranes, including fragments with the N-terminal 178 or N-terminal 125 amino acids of human MLKL (Xia et al. 2016, Cell Res 26:517-528). The 4HD domain (sometimes referred to as amino acids 1-124, 1-125, 1-179, 1-180, etc.), but not the pseudokinase domain (sometimes referred to as amino acids 179-464, 180-464, etc.) was shown to be required for both membrane association and cell-killing activity (Hildebrand et al. 2014, Proc Natl Acad Sci USA 111:15072-15077).

Based on the above in vitro data (no information available on in vivo/in situ responses) combined with the teachings of the current invention, all variants of essentially full-length MLKL and all truncated variants (such as the truncated human isoform), possibly combined with amino acid variations allowed in the full-length MLKL, still capable of forming octamers and/or still capable of disrupting membranes (such as in liposomes) are considered MLKL variants capable of replication the technical effects according to the invention as claimed. Assays for determining the ability of MLKL variants (comprising amino acid variation or mutation (1 or more) relative to full-length MLKL or relative to truncated MLKL or MLKL fragment) to disrupt membranes or to form octamers are available as described in the references cited above.

Experimental work outlined herein lead to several surprising and fully unexpected results. First: the results indicate that both the 4HD domain fragment of MLKL, the pseudokinase domain of MLKL, as well as a phosphomimetic S345D mutant version of full-length MLKL and a non-phosphorylatable S345A mutant version of full-length MLKL all are capable of retarding tumor growth in an in vivo mouse tumor model (the S345D and S345A mutations are relative to murine wild-type MLKL; see SEQ ID NO:1); fragments and variants of MLKL protein other than those described above may thus likewise replicate the therapeutic effects initially seen with wild-type full-length MLKL. Secondly, the results surprisingly indicate that the anti-tumor and adaptive immune response-inducing activities of MLKL appear to be independent of RIPK3 activity. Finally, the anti-tumor and adaptive immune response-inducing activities of MLKL were shown to be stronger than when RIPK3 is administered in a similar way.

In any of the above aspects and embodiments, the tumor, cancer or neoplasm; or the tumor, cancer or neoplasm cell may be deficient in RIPK3.

Immunotherapeutic Treatment and Immune Response

Immunotherapeutic treatment as used herein refers to the reactivation and/or stimulation and/or reconstitution of the immune response of a mammal towards a condition such as a tumor, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumor or anti-neoplasm immune response; adaptive immune response to the tumor, cancer or neoplasm).

Not all insults capable of inducing death of tumor or cancer cells result in immunogenic cell death (ICD) of these tumor or cancer cells, thus not reactivating and/or stimulating and/or reconstituting the immune response of a mammal towards these tumor or cancer cells. Necrosis of the murine cancer cell line TC-1 induced by freeze/thawing for example does not generate ICD. The same cell line treated with TSZ (a combination of tumor necrosis factor-α, the caspase-9 activator SMAC and the pan-caspase inhibitor z-VAD-FMK) induces necrosis and TSZ-treated TC-1 cells are able to induce a protective anticancer immune response which was abolished by knocking out Ripk3 or MlkI. The anthracycline mitoxantrone (MTX) or oxaliplatin (OXA) induced cell death of wild-type, Ripk3-deficient and MlkI-deficient TC-1 cells, but only MTX- or OXA-treated wild-type TC-1 cells induced a protective ICD response (Yang et al. 2016, Oncoimmunology 5:e1149673). Administration of/vaccination with necroptotic tumor cells has been shown to induce anti-tumor immunity (Aaes et al. 2016, Cell Rep 15:274-287).

Necroptosis is often impaired during tumorigenesis, and induction of necrosis is assumed to exert a bimodal action: direct elimination of tumor cells at the one hand, and indirect elimination of tumor cells by invoking (reactivating, stimulating and/or reconstituting) the host's innate and adaptive immune response to the tumor cells. Such adaptive immune response is aiding in clearing the tumor cells (Meng et al. 2016, Oncotarget 7:57391-57413). Although MLKL is known to be involved in the necroptosis pathway, Induction of ICD by MLKL (such as administered as nucleic acid therapy or as protein) has so far never been demonstrated neither in vitro nor in vivo. In view of the requirement for MLKL to be phosphorylated by RIPK3 to induce necroptosis, it is moreover all the more surprising that such therapy is effective in RIPK3-deficient cells in vitro and in vivo (CT26 cells are RIPK3 deficient: Aaes et al. 2016, Cell Rep 15:274-287; B16 cells are RIPK3 deficient: Morgan & Kim 2015, BMB Rep 48:303-312). Therefore, in any of the aspects and embodiments of the invention, the tumor, cancer or neoplasm cell may in particularly be deficient in receptor-interacting serine/threonine protein kinase 3 (RIPK3).

When the adaptive immune response to the cancer, tumor or neoplasm cells is mediated by or is involving cells of the immune system (such as one or more of CD4+ T-cells, CD8+T-cells, antigen-presenting cells (APCs), dendritic cells (DCs, e.g. cDC1 and/or cDC2)) the (adaptive) immune response is a (adaptive) cellular immune response.

Inducing an (adaptive) immune response to tumor, cancer or neoplasm cells herein refers to a process that (re-)activates the host's immune response to the tumor, cancer or neoplasm; the induced (adaptive) immune response can, but does not need to be sufficient to fully eradicate a primary tumor, cancer or neoplasm. Likewise, the induced (adaptive) immune response can, but does not need to be sufficient to treat, suppress or inhibit secondary tumor, cancer or neoplasm growth and/or tumor, cancer or neoplasm metastasis. Independent thereof, the induced (adaptive) immune response is useful in (a method for) immunotherapeutic treatment of a tumor, cancer, or neoplasm.

“Treatment”/“treating” refers to any rate of reduction, retardation or inhibition of the progress of the disease or disorder compared to the progress or expected progress of the disease or disorder when left untreated. More desirable, the treatment results in no/zero progress of the disease or disorder (i.e. full inhibition or full inhibition of progression) or even in any rate of regression of the already developed disease or disorder. “Suppressing” can in this context be used as alternative for “treating”.

Necroptosis

Several mechanisms of programmed cell death (PCD) exist in nature, of which apoptosis is probably currently best characterized. A critical factor in the apoptosis process is the presence of active caspases such as caspase-8 or -3 for example. Necroptosis, characterized by organelle swelling and membrane integrity loss and now also recognized as a mechanism of PCD, is in contrast to apoptosis independent of caspase activity but relies on RIPK3 activity for phosphorylation of the pseudokinase MLKL in the necrosome. Apoptotic cells produce “find me” and “eat me” signals to enable fast phagocytosis by macrophages, thus suppressing inflammation (as required for normal development and homeostasis). Although necroptosis is considered to trigger an inflammatory response, it was recently shown that the initial phase of necroptosis (prior to actual cell death) may be an immunologically silent phase in producing “find me” and “eat me” signals characteristic for apoptosis, concomitant with phagocytosis of “necroptotic bodies”. The process was shown to involve phosphorylated MLKL (Zargarian et al. 2017, PLos Biol 15:e2002711). Necroptotic-like death of tumor cells refers to a PCD process of tumor cells that has the hallmarks of necroptosis, i.e., at least is characterized by organelle swelling and loss of membrane integrity. The occurrence of such process can be validated by means of administering a candidate necroptosis-inducing agent to e.g. in vitro cultured tumor cells.

Inducing necroptosis or necroptotic-like death of tumor, cancer or neoplasm cells herein refers to a process that (re-)activates necrosis of tumor, cancer or neoplasm cells; the induced necroptosis or necroptotic-like death of tumor, cancer or neoplasm cells can, but does not need to be sufficient to fully eradicate a primary tumor, cancer or neoplasm. Likewise, the induced necroptosis or necroptotic-like death of tumor, cancer or neoplasm cells can, but does not need to induce ICD sufficient to fully eradicate a primary tumor, cancer or neoplasm. Likewise, the induced necroptosis or necroptotic-like death of tumor, cancer or neoplasm cells can, but does not need to be sufficient to treat, suppress or inhibit secondary tumor, cancer or neoplasm growth and/or tumor, cancer or neoplasm metastasis. Likewise, the induced necroptosis or necroptotic-like death of tumor, cancer or neoplasm cells can, but does not need to be to induce ICD sufficient to treat, suppress or inhibit secondary tumor, cancer or neoplasm growth and/or tumor, cancer or neoplasm metastasis. Independent thereof, the induced necroptosis or necroptotic-like death of tumor, cancer or neoplasm cells is useful in (a method of) treatment, such as immunotherapeutic treatment of a tumor, cancer, or neoplasm; and useful in (a method of) treatment, suppression or inhibition of secondary tumor, cancer or neoplasm growth and/or tumor, cancer or neoplasm metastasis.

Tumor, Cancer, Neoplasm

The terms tumor and cancer are sometimes used interchangeably but can be distinguished from each other. A tumor refers to “a mass” which can be benign (more or less harmless) or malignant (cancerous). A cancer is a threatening type of tumor. A tumor is sometimes referred to as a neoplasm: an abnormal cell growth, usually faster compared to growth of normal cells. Benign tumors or neoplasms are non-malignant/non-cancerous, are usually localized and usually do not spread/metastasize to other locations. Because of their size, they can affect neighboring organs and may therefore need removal and/or treatment. A cancer, malignant tumor or malignant neoplasm is cancerous in nature, can metastasize, and sometimes re-occurs at the site from which it was removed (relapse).

The initial site where a cancer starts to develop gives rise to the primary cancer. When cancer cells break away from the primary cancer (“seed”), they can move (via blood or lymph fluid) to another site even remote from the initial site. If the other site allows settlement and growth of these moving cancer cells, a new cancer, called secondary cancer, can emerge (“soil”). The process leading to secondary cancer is also termed metastasis, and secondary cancers are also termed metastases. For instance, liver cancer can arise as primary cancer, but can also be a secondary cancer originating from a primary breast cancer, bowel cancer or lung cancer; some types of cancer show an organ-specific pattern of metastasis.

Most cancer deaths are in fact caused by metastases, rather than by primary tumors (Chambers et al. 2002, Nature Rev Cancer 2:563-572).

In 2012, cancer was the second leading cause of deaths in the USA, but coming very close to the first leading cause being heart diseases. For 2016, the estimated number of new cancer cases (both sexes where relevant) in the USA are, ranked from highest to lowest, breast cancer, lung and bronchus cancer, prostate cancer, colon cancer, skin melanoma and urinary bladder cancer, non-Hodgkin lymphoma, thyroid cancer and kidney and renal pelvis cancer, uterine corpus cancer, pancreas cancer, and rectum cancer and liver and intrahepatic bile duct cancer; jointly about 1,293 million new cases (circa 77% of total expected new cases) (Siegel et al. 2016, CA Cancer. Clin 66:7-30). These, including all other possible types of cancer are targets for the treatment as experimentally supported herein.

Nucleic Acid Therapy

Interest in nucleic acid-based therapies has increased over the years. Key in (viral) DNA-based therapy is the presence in the vector of transcription signals enabling production of translatable mRNA in the target cell. In view of concerns regarding the safety of DNA and vector-based therapy, the use of antigen-encoding translatable (m)RNA for vaccination has gained traction. Compared to viral vectors or plasmid DNA, (m)RNA-based therapy present several advantages. In lacking the ability to integrate in the host genome, it is presumed to be much safer (no inadvertent mutations, and transient expression of the encoded protein leading to controlled antigen exposure and minimization of tolerance induction). Potentially foreign sequences such as plasmid backbone or viral promotors are not required, reducing the risk in raising an immune response. Further, it offers the possibility to transfect slow or non-dividing cells as RNA does not need to cross the nuclear barrier for protein expression. Especially in the context of the current invention wherein it is the purpose to drive tumor cells into necroptosis, these potential drawbacks of DNA or (viral)vector-based are less of a concern. Adaptation to result in transient, or in the alternative, inducible expression of the target protein and/or targeted delivery of the nucleic acid to the tumor, cancer or neoplasm may nevertheless be of use. Direct intra-tumor, intra-cancer or intra-neoplasm delivery (e.g. upon tumor, cancer or neoplasm biopsy or upon surgical debulking of a tumor, cancer or neoplasm) represents a further method of targeted delivery.

Methods for administering nucleic acids include methods applying non-viral (DNA or RNA) or viral nucleic acids (DNA or RNA viral vectors). Methods for non-viral gene therapy include the injection of naked DNA (circular or linear), electroporation, the gene gun, sonoporation, magnetofection, the use of oligonucleotides, lipoplexes (e.g. complexes of nucleic acid with DOTAP or DOPE or combinations thereof, complexes with other cationic lipids), dendrimers, viral-like particles, inorganic nanoparticles, hydrodynamic delivery, photochemical internalization (Berg et al. 2010, Methods Mol Biol 635:133-145) or combinations thereof.

Many different vectors have been used in human nucleic acid therapy trials and a listing can be found on http://www.abedia.com/wiley/vectors.php. Currently the major groups are adenovirus or adeno-associated virus vectors (in about 21% and 7% of the clinical trials, respectively), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses) are used in nucleic acid therapy and are not excluded in the context of the current invention.

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Tumor-, cancer- or neoplasm-targeting strategies may also be applied to the nucleic acid (nucleic acid combined with tumor-, cancer-, or neoplasm-targeting moiety); these include passive targeting (mostly achieved by adapted formulation) or active targeting (e.g. by coupling a nucleic acid-comprising nanoparticle with folate or transferrin, or with an aptamer or antibody binding to an target cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

Any other modification of the DNA or RNA to enhance efficacy of nucleic acid therapy is likewise envisaged to be useful in the context of the applications of the nucleic acid encoding a MLKL protein (MLKL; in particular wild-type MLKL), a variant of MLKL, a fragment of MLKL, or a fragment of a variant of MLKL (in particular the variant or fragment of MLKL is a membrane-permeabilizing variant of MLKL or a membrane-permeabilizing fragment of MLKL) as outlined herein. The enhanced efficacy can reside in enhanced expression, enhanced delivery properties, enhanced stability and the like. The applications of the nucleic acid encoding a MLKL protein (MLKL; in particular wild-type MLKL), a variant of MLKL, a fragment of MLKL, or a fragment of a variant of MLKL (in particular the variant or fragment of MLKL is a membrane-permeabilizing variant of MLKL or a membrane-permeabilizing fragment of MLKL) as outlined herein may thus rely on using a modified nucleic acid as described above, or as described in the next section. It is also conceivable to deliver nucleic acid encoding a MLKL protein (MLKL; in particular wild-type MLKL), a variant of MLKL, a fragment of MLKL, or a fragment of a variant of MLKL (in particular the variant or fragment of MLKL is a membrane-permeabilizing variant of MLKL or a membrane-permeabilizing fragment of MLKL) in an oncolytic virus, or in combination with an oncolytic virus. Oncolytic viruses are reviewed in e.g. Kaufman et al. 2015 (Nat Rev Drug Discov 14:642-662).

Hypoinflammatory Nucleic Acids

A known problem with e.g. adenoviral nucleic acid therapy is its triggering of an inflammatory response. Less inflammatory (hypoinflammatory) helper-dependent or gutless adenovirus vectors, can alternatively be used as hypoinflammatory adenoviral vector for nucleic acid therapy. Other solutions include covalent modification of the viral capsid proteins (e.g. by PEGylation), modifying the adenoviral fiber knob (composition), vector encapsulation in a polymer, and/or serotype switching or reverting to non-human adenoviral vectors (e.g. Ahi et al. 2011, Curr Gene Ther 11:307-320).

Naked DNA nucleic acid therapy can likewise provoke inflammatory responses. Linear DNA from which the bacterial backbone sequences were removed was reported to be less inflammatory (hypoinflammatory) than linear DNA comprising the bacterial backbone sequences and to be less inflammatory than circular DNA (Zhu et al. 2009, Biomed Pharmacother 63:129-135). Reducing the amount of unmethylated CpG motifs or sequential injection of cationic liposomes followed by naked plasmid DNA are other alternatives to arrive at hypoinflammatory DNA therapy (Niidome & Huang 2002, Gene Therapy 9:1647-1652).

In case of RNA-based expression constructs, it was also reported that they can induce inflammatory immune responses which could ameliorate their efficacy. Kariko et al. 2005 (Immunity 23:165-175) established that modified to heavily modified eukaryotic RNA is not immunostimulatory compared to nearly unmodified RNA (eukaryotic or other). On the other hand, mRNA lacking poly(A)-tail is also immunostimulatory (even from a eukaryotic source). This led to the suggestion of including naturally occurring modified nucleosides (more than 100 exist, a list is available on http://mods.rna.albany.edu/mods/), such as 5-methylcytidine and pseudouridine, in therapeutic RNA (Pollard et al. 2013 Mol Ther 21:251-259). Hypoinflammatory RNA as referred to herein is heterologous RNA constructed such as to minimize potential inflammatory responses by including naturally occurring modified nucleosides wherein the modified nucleosides are preferably unique to and frequently used in RNA of the species in which the heterologous hypoinflammatory RNA is to be administered.

In view of the explanation of nucleic acid therapy and hypo-inflammatory nucleic acids, the described aspects and embodiments of the invention can be refined. Thus in any of the aspects and embodiments of the invention, the nucleic acid encoding a MLKL protein (MLKL; in particular wild-type MLKL), a variant of MLKL, a fragment of MLKL, or a fragment of a variant of MLKL (in particular the variant or fragment of MLKL is a membrane-permeabilizing fragment of MLKL or a membrane-permeabilizing variant of MLKL) may be DNA or RNA. In case of it being DNA, it may be naked DNA, plasmid DNA, DNA included in a viral vector, or complexed DNA (e.g. complexed with lipids or nanomaterials). In case of it being RNA, it may be naked RNA, RNA included in a viral vector, mRNA, or complexed (m)RNA (e.g. complexed with lipids or nanomaterials). Combinations (in any order or timing) of any of these are also envisaged by the current invention.

If, in any of the aspects and embodiments of the invention, the nucleic acid encoding a MLKL protein (MLKL; in particular wild-type MLKL), a variant of MLKL, a fragment of MLKL, or a fragment of a variant of MLKL (in particular the variant or fragment of MLKL is a membrane-permeabilizing fragment of MLKL or a membrane-permeabilizing variant of MLKL) is mRNA, the mRNA may comprise elements such as a 5′ cap and/or a 3′ poly(A)tail and/or a 5′ untranslated region and/or a 3′ untranslated region.

In any of the aspects and embodiments of the invention, the nucleic acid a encoding MLKL protein (MLKL; in particular wild-type MLKL), a variant of MLKL, a fragment of MLKL, or a fragment of a variant of MLKL (in particular the variant or fragment of MLKL is a membrane-permeabilizing fragment of MLKL or a membrane-permeabilizing variant of MLKL) may be a hypo-inflammatory nucleic acid or modified nucleic acid.

In order to obtain the outlined clinical effects, the nucleic acid a encoding MLKL protein (MLKL; in particular wild-type MLKL), a variant of MLKL, a fragment of MLKL, or a fragment of a variant of MLKL (in particular the variant or fragment of MLKL is a membrane-permeabilizing fragment of MLKL or a membrane-permeabilizing variant of MLKL) for use (in methods) outlined in any of the aspects and embodiments of the invention is administered to the tumor, cancer or neoplasm. Such administration may for instance be by intra-tumor, intra-cancer or intra-neoplasm delivery, or may be for instance be remote administration of the nucleic acid (administration remotely from the tumor, cancer or neoplasm), optionally combined with for instance a tumor-, cancer- or neoplasm-targeting moiety.

In any of the aspects and embodiments of the invention, the nucleic acid encoding a MLKL protein (MLKL; in particular wild-type MLKL), a variant of MLKL, a fragment of MLKL, or a fragment of a variant of MLKL (in particular the variant or fragment of MLKL is a membrane-permeabilizing fragment or variant of MLKL) may be designed such that expression in the tumor, cancer or neoplasm of the mixed-lineage kinase domain-like protein (MLKL) protein (MLKL; in particular wild-type MLKL), a variant of MLKL, a fragment of MLKL, or a fragment of a variant of MLKL (in particular the variant or fragment of MLKL is a membrane-permeabilizing variant of MLKL or a membrane-permeabilizing fragment of MLKL) is transient, or, in the alternative, inducible.

Protein Transduction/Transfection/Delivery into Cells

Transduction of a protein, such as a MLKL protein (full-length or fragment of a wild-type or variant MLKL), into live cells is a further possibility to obtain the therapeutic effects of MLKL as described herein. One way of getting a protein into a cell is via coupling the protein of interest with a protein transduction domain (PTD) or cell-penetrating peptide (CPP) wherein the CPP is providing the ability to carry cell-impermeable molecules inside live cells. As discussed by Schwarze et al. 2000 (Trends Cell Biol 10:290-295), this technology is also applicable in vivo. One potential hurdle that may need to be overcome is entrapment of the transduced protein in endosomes. Several strategies to overcome this have been discussed by Erazo-Oliveras et al. 2012 (Pharmaceuticals 5:1177-1209) and include the use of multivalent CPPs, of pH-dependent membrane active peptides (PMAPs, e.g. the HA2 fusion peptide, a 23-amino acid peptide of the N-terminus of the hemagglutinin HA2 subunit of the influenza virus X31), of PMAP-CPP chimeras, or of CPP-mediated photochemical internalization (excitation of a photosensitizer coupled to a CPP leads to endosomolytic activity possibly mediated by reactive oxygen species). Another solution to overcome endosome entrapment is the co-incubation of the to-be transduced protein with an endosomolytic agent such as a dimerized from of the cell-penetrating peptide TAT, which may even obviate the need for coupling of the protein of interest to a PTD/CPP (Erazo-Oliveras et al. 2014, Nat Methods-11:861-867).

A further methodology for intracellular delivery of a protein (or other macromolecule) of interest is TOP (induced transduction by osmocytosis and propanebetaine), as described by D'Astolfo et al. 2015 (Cell 161:674-690). Other methods rely on diffusion of large cargo, such as a protein of interest, through transient openings in the cell membrane caused by electroporation or laser pulsing (Wu et al. 2015, Nat Methods 12:439-443), or by microfluidic-based cell squeezing (Sharei et al. 2013, Proc Natl Acad Sci USA 110:2082-2087). Further alternatives rely on enhancing endocytosis by packaging of a protein of interest into nanoparticles or nanocapsules (e.g. Slowing et al. 2007, J Am Chem Soc 129:8845-8849), on fusing with a supercharged protein (Thompson et al. 2012, Methods Enzymol 503:293-319), or on microinjection. Bulkescher et al. 2017 (Genome Res 27:1752-1758) disclosed a solid-phase reverse transfection method of cellular delivery of large biomolecules such as proteins, based on surface coating with mixtures containing transfection reagent, protein, and the carrier molecules. Yet a further method relies on photoporation relying on gold-coated nanoparticles (Vermeulen et al. 2018, Int J Mol Sci 19:2400).

Administration of a MLKL protein to a mammal harboring a tumor, cancer or neoplasm may for instance be by intra-tumor, intra-cancer or intra-neoplasm delivery. The administration of MLKL protein may alternatively be remote (administration remotely from the tumor, cancer or neoplasm); in this case the MLKL protein is optionally combined with or (recombinantly or non-recombinantly) fused to for instance a tumor-, cancer- or neoplasm-targeting moiety.

Production of a protein of interest, whether or not fused to e.g. a CPP, PTD, PMAP, etc., can be performed recombinantly. Such recombinant expression of a protein of interest (such as MLKL protein (wild-type or containing a variation or mutation), a fragment of MLKL protein, a variant MLKL protein) may be in a prokaryotic host (e.g. Escherichia coli) although there may be an advantage to produce protein such as by expression in eukaryotic cells (e.g. mammalian cell line such as CHO or COS, insect cells, yeast cells such as Pichia pastoris). Such cell lines may be capable of expressing the protein of interest either in a transient, inducible, or constitutive fashion. Other recombinant production systems include cultured plant cells, whole plants, duckweed, and algae.

Receptor-Interacting Serine/Threonine-Protein Kinase 3

Receptor-interacting serine/threonine-protein kinase 3 is also known as RIPK3 or RIP3. The human RIPK3 protein is a 518-amino acid protein (Genbank Accession No. NP 006862; murine isoforms of RIPK3 protein: Genbank Accession Nos. NP 001157579, NP 001157580, NP 064339). At least two splice variants of human RIPK3 have been identified (Yang et al. 2005, Biochem Biophys Res Commun 332:181-187). The loss of RIPK3 expression in many cancer cells and the effect thereof on repression of TNF-α or chemotherapeutic-induced necrosis is documented in He et al. 2009 (Cell 137:1100-1111) and Koo et al. 2015 (Cell Res 25:707-725).

Pharmaceutical Compositions

The therapeutic modality of the current invention, i.e. either of a MLKL protein or of a nucleic acid encoding a MLKL protein (as described hereinabove) may be comprised in a composition (MLKL protein composition/MLKL-encoding nucleic acid composition). In particular the composition is a pharmaceutical composition, in particular in a pharmaceutically acceptable composition. The therapeutic modality of the current invention may be part of a (pharmaceutical) kit, such as a separate, individual, or separately packaged pharmaceutical composition. In some instances, such (pharmaceutical) kit may comprise one or more further therapeutic modalities (active ingredients, medicaments) in the form of one or more separate, individual, or separately packaged pharmaceutical composition(s).

A pharmaceutical composition in general comprises, besides the active ingredient(s) or medicament(s), components useful in stabilizing, storing and/or administering the active ingredient or medicament. Such components are commonly referred to herein as “pharmaceutical carrier” or “pharmaceutically acceptable carrier”.

Combination Therapy

The therapeutic modality of the current invention is an MLKL protein, a variant MLKL protein, a fragment of MLKL protein, or a fragment of a variant MLKL protein; or is a nucleic acid encoding a MLKL protein (MLKL; in particular wild-type MLKL), a variant of MLKL, a fragment of MLKL, or a fragment of a variant of MLKL; all as described hereinabove. The therapeutic modality may be comprised in a pharmaceutical composition as described hereinabove. The scope of the therapeutic modality of the current invention can be further expanded as it may in itself consist of a combination (in any way or form; simultaneously or in any order) of for instance a nucleic acid encoding a MLKL protein (or fragment or variant thereof, see above) and a MLKL protein (or fragment or variant thereof, see above).

The therapeutic modality of the current invention, or the (pharmaceutical) composition comprising it, can be combined (in any way or form; simultaneously or in any order) with one or more further antitumor, anticancer or antineoplastic therapy in a combination therapy. Several types of antitumor, anticancer or antineoplastic therapy are listed hereunder. It will be clear, however, that none of these lists is meant to be exhaustive and is included merely for illustrative purposes. In one embodiment, the combination involves combination of separate or individual or separately packaged pharmaceutical compositions, one of these compositions comprising the therapeutic modality of the current invention. In another embodiment, the therapeutic modality of the current invention is a pharmaceutical composition itself further comprising one or more active ingredients or medicaments different from the therapeutic modality of the current invention.

As referred to hereinabove, administration of the therapeutic modality of the current invention (whether or not already comprising a further therapeutic agent), or the (pharmaceutical) composition comprising it, could for instance occur at the time of surgical removal of the (primary or secondary) tumor, cancer or neoplasm (debulking the tumor, cancer or neoplasm mass) although it may be preferred to perform the administration of the therapeutic modality of the current invention, or the (pharmaceutical) composition comprising it, prior to surgical removal in order to provide sufficient time and/or sufficient (remaining) tumor, cancer or neoplasm cells for the immunotherapeutic potential of the therapeutic modality of the current invention to develop. In many, if not all, cases a biopsy is taken of a tumor, cancer or neoplasm; as this procedure provides access to the tumor, cancer or neoplasm, the therapeutic modality of the current invention, or the (pharmaceutical) composition comprising it, could be administered at this timepoint. Combination of administration of the therapeutic modality of the current invention, or of the (pharmaceutical) composition comprising it, with radiation therapy or chemotherapy can also be envisaged.

The above approach of administration of the therapeutic modality of the current invention, or of the (pharmaceutical) composition comprising it, to induce tumor antigen-specific T cell responses early on in cancer patients has other benefits. By triggering a protective adaptive immune response, time can be bought for subsequent characterization of the tumor mutanome (e.g. by exome sequencing and bio-informatic prediction tools), and the primed immune response induced by treatment with (a composition comprising) the MLKL protein, a variant MLKL protein, a fragment of MLKL protein, or a fragment of a variant MLKL protein; or by treatment with (a composition comprising) a nucleic acid encoding a MLKL protein (MLKL; in particular wild-type MLKL), a variant of MLKL, a fragment of MLKL, or a fragment of a variant of MLKL (all as described hereinabove) can subsequently be boosted with, for example, a follow up therapeutic vaccination based on the identified neo-epitopes. A combination therapy as envisaged herein thus can include one or more steps such as characterization of the tumor mutanome (compared to normal or healthy cells or non-tumor cells), designing a (personalized) neo-epitope vaccine, designing CAR T-cells (CAR: chimeric antigen receptors, also known as chimeric immunoreceptors, chimeric T cell receptors or artificial T cell receptors), administration of a neo-epitope vaccine, administration of CAR T-cells.

As referred to hereinabove, the lack or suppression of necrosis of tumor or cancer cells may compromise the efficacy of anticancer agents (Meng et al. 2016, Oncotarget 7:57391-57413). This is a further reason for possibly including the therapeutic modality of the current invention (simultaneously or in any order) with one or more other antitumor, anticancer or antineoplastic agent(s) in a combination therapy.

Without being exhaustive, antitumor, anticancer or antineoplastic agents include alkylating agents (nitrogen mustards: melphalan, cyclophosphamide, ifosfamide; nitrosoureas; alkylsulfonates; ethyleneimines; triazene; methyl hydrazines; platinum coordination complexes: cisplatin, carboplatin, oxaliplatin), antimetabolites (folate antagonists: methotrexate; purine antagonists; pyrimidine antagonists: 5-fluorouracil, cytarabibe), natural plant products (Vinca alkaloids: vincristine, vinblastine; taxanes: paclitaxel, docetaxel; epipodophyllotoxins: etoposide; camptothecins: irinotecan), natural microorganism products (antibiotics: doxorubicin, bleomycin; enzymes: L-asparaginase), hormones and antagonists (corticosteroids: prednisone, dexamethasone; estrogens: ethinyloestradiol; antiestrogens: tamoxifen; progesteron derivative: megestrol acetate; androgen: testosterone propionate; antiandrogen: flutamide, bicalutamide; aromatase inhibitor: letrozole, anastrazole; 5-alpha reductase inhibitor: finasteride; GnRH analogue: leuprolide, buserelin; growth hormone, glucagon and insulin inhibitor: octreotide). Other antineoplastic or antitumor agents include hydroxyurea, imatinib mesylate, epirubicin, bortezomib, zoledronic acid, geftinib, leucovorin, pamidronate, and gemcitabine.

Without being exhaustive, antitumor, anticancer or antineoplastic antibodies (antibody therapy) include rituximab, bevacizumab, ibritumomab tiuxetan, tositumomab, brentuximab vedotin, gemtuzumab ozogamicin, alemtuzumab, adecatumumab, labetuzumab, pemtumomab, oregovomab, minretumomab, farletuzumab, etaracizumab, volociximab, cetuximab, panitumumab, nimotuzumab, trastuzumab, pertuzumab, mapatumumab, denosumab, and sibrotuzumab.

A particular class of antitumor, anticancer or antineoplastic agents are designed to stimulate the immune system (immune checkpoint or other immunostimulating therapy). These include so-called immune checkpoint inhibitors or inhibitors of co-inhibitory receptors and include PD-1 (Programmed cell death 1) inhibitors (e.g. pembrolizumab, nivolumab, pidilizumab), PD-L1 (Programmed cell death 1 ligand) inhibitors (e.g. atezolizumab, avelumab, durvalumab), CTLA-4 (Cytotoxic T-lymphocyte associated protein 4; CD152) inhibitors (e.g. ipilimumab, tremelimumab) (e.g. Sharon et al. 2014, Chin J Canc 33:434-444). PD-1 and CTLA-4 are members of the immunoglobulin superfamily of co-receptors expressed on T-cells. Inhibition of other co-inhibitory receptors under evaluation as antitumor, anticancer or antineoplastic agents include inhibitors of Lag-3 (lymphocyte activation gene 3), Tim-3 (T cell immunoglobulin 3) and TIGIT (T cell immunoglobulin and ITM domain) (Anderson et al. 2016, Immunity 44:989-1004). Stimulation of members of the TNFR superfamily of co-receptors expressed on T-cells, such as stimulation of 4-1BB (CD137), OX40 (CD134) or GITR (glucocorticoid-induced TNF receptor family-related gene), is also evaluated for antitumor, anticancer or antineoplastic therapy (Peggs et al. 2009, Clin Exp Immunol 157:9-19). The list of such agent stimulating the immune system is ever growing.

Further antitumor, anticancer or antineoplastic agents include immune-stimulating agents such as—or neo-epitope cancer vaccines (neo-antigen or neo-epitope vaccination; based on the patient's sequencing data to look for tumor-specific mutations, thus leading to a form of personalized immunotherapy; Kaiser 2017, Science 356:112; Sahin et al. 2017, Nature 547:222-226) and some Toll-like receptor (TLR) ligands (Kaczanowska et al. 2013, J Leukoc Biol 93:847-863).

Yet further antitumor, anticancer or antineoplastic agents include oncolytic viruses (oncolytic virus therapy) such as employed in oncolytic virus immunotherapy (Kaufman et al. 2015, Nat Rev Drug Discov 14:642-662), any other cancer vaccine (cancer vaccine administration; Guo et al. 2013, Adv Cancer Res 119:421-475), and any other anticancer nucleic acid therapy (wherein “other” refers to it being different from therapy with a therapeutic modality of the current invention as outlined hereinabove).

Therefore, in any of the aspects and embodiments of the invention, the MLKL protein, variant MLKL protein, fragment of MLKL protein, or fragment of a variant MLKL protein; or the nucleic acid encoding a MLKL protein (MLKL; in particular wild-type MLKL), variant of MLKL, fragment of MLKL, or fragment of a variant of MLKL; or the therapeutic modality of the invention; or the (pharmaceutical) composition comprising a therapeutic modality—all as described hereinabove—may be further combined with another therapy against the tumor, cancer or neoplasm. Such other or further therapies include for instance surgery, radiation, chemotherapy, immune checkpoint or other immunostimulating therapy, neo-antigen or neo-epitope vaccination, cancer vaccine administration, oncolytic virus therapy, antibody therapy, or any other nucleic acid therapy targeting or treating the tumor, cancer or neoplasm.

In case of combination therapy, the nucleic acid encoding a MLKL or the isolated MLKL protein may be provided as a separate or individual or separately packaged pharmaceutical composition, with the further therapy or therapies (in case not being surgery or radiation) being provided in one or more further separate or individual or separately packaged pharmaceutical composition or compositions. Alternatively, the nucleic acid encoding a MLKL or the isolated MLKL protein may be provided as a separate or individual or separately packaged pharmaceutical composition, itself comprising a further therapeutic agent or itself comprising more than one further therapeutic agents (in case the further therapy is not surgery or radiation). In this alternative, further additional therapy or therapies (different from surgery or radiation) can be provided in one or more additional separate or individual or separately packaged pharmaceutical composition or compositions.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

The term “defined by SEQ ID NO:X” as used herein refers to a biological sequence consisting of the sequence of amino acids or nucleotides given in the SEQ ID NO:X. For instance, an antigen defined in/by SEQ ID NO:X consists of the amino acid sequence given in SEQ ID NO:X. A further example is an amino acid sequence comprising SEQ ID NO:X, which refers to an amino acid sequence longer than the amino acid sequence given in SEQ ID NO:X but entirely comprising the amino acid sequence given in SEQ ID NO:X (wherein the amino acid sequence given in SEQ ID NO:X can be located N-terminally or C-terminally in the longer amino acid sequence, or can be embedded in the longer amino acid sequence), or to an amino acid sequence consisting of the amino acid sequence given in SEQ ID NO:X.

The group of mammals includes, besides humans, mammals such as primates, cattle, horses, sheep, goats, pigs, rabbits, mice, rats, guinea pigs, llama's, dromedaries and camels.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

The content of the documents cited herein are incorporated by reference.

### Examples

### A. Murine Tumor Cell Lines

A.1. Materials and Methods

A.1.1. Cell Line and Culture Conditions

Cell culture experiments were performed using the murine tumor cell line B16, B16F10 or CT26. Cells were cultured in Dulbecco's modified Eagle's medium (DMEM) supplemented with 10% fetal calf serum, 2 mM L-glutamine, 0.4 mM Na-pyruvate, non-essential amino acids, 100 U/ml penicillin and 0.1 mg/ml streptomycin at 37° C. in a humidified atmosphere containing 5% CO₂. Murine tumor cells used were melanoma cell lines (B16, B16-OVA, B16-F10) and colon carcinoma cells (CT-26). Human melanoma cell lines (501 Mel, BLM, SK-mel28) and early passage cultures (M018017 and M000921) were kindly provided by Dr. Geert Berx from Ghent University. RL cells were purchased from the American Type Culture Collection (ATCC) and cultured in conditions specified by the manufacturer. All cells used were tested for mycoplasma.

A.1.2. Production of In Vitro Transcribed mRNA

The coding information for Fluc, mouse tBid, mouse MLKL and human MLKL were cloned into the in-house generated plasmid vector pIVTstab that contains a T7 promoter, 5′ and 3′ untranslated region (UTR) of human β globulin (HBB) and a poly A₆₀ tail. The mRNA expression plasmids pIVTstab-GFP, pIVTstab-Luc, pIVTstab-tBid and pIVTstab-MLKL were all propagated in E. coli MC1061 competent cells (Stratagene, La Jolla, Calif., USA) and purified using endotoxin-free QIAGEN-tip500 columns (Qiagen, Chatsworth, Calif., USA). The MLKL and tBid encoding plasmids were linearized with PstI (New England Biolabs, MA, USA) whereas the OVA, GFP and luciferase encoding plasmids were linearized with SpeI (New England Biolabs, MA, USA). The linearized plasmids were purified using a PCR purification kit (Roche, Upper Bavaria, Germany). The mRNA was transcribed using the T7 mMessage Machine Kit (Ambion, Austin, Tx, USA) according to the manufacturer's instruction. 5-methylcytidine and pseudouridine (TriLink, San Diego, Calif., USA) was used in the transcription reactions instead of respectively cytidine and uridine. The in vitro transcribed mRNA was purified by lithium chloride precipitation and the mRNA was simultaneously capped and 2′-O-methylated to synthesize Cap 1 RNA from uncapped RNA using the ScriptCap m⁷G Capping system kit together with the ScriptCap 2′-O-methyltransferase kit (Ambion, Austin, Tx, USA) according to the manufacturer's instruction.

A.1.3. Transfection In Vitro

Cells were plated 24 hours before transfection in a six-well or 96-well plate at 10⁶ or 10⁴ cells/well, respectively. One million B16 cells were transfected with 1 μg of mRNA complexed with Lipofectamine® RNAiMAX (Life Technologies, Ghent; Belgium) diluted in OptiMem to obtain a total volume of 300 μl according to the manufacturer's instruction. The transfection mix was added to the cells and cells were incubated at 37° C., 5% carbon dioxide during a time period depending on the experiment. Transfection efficiency was evaluated by measuring uptake of cy-5 labelled eGFP mRNA and onset of translation of the transfected mRNA by determining GFP fluorescence at different time points after transfection using flow cytometry. The flow cytometric experiment was performed on a triple-laser (B-V-R) LSR-II (Becton Dickinson, San Jose, Calif., USA) and analyzed with FlowJo (Treestar, Oreg.)

A.1.4. Cell Death Assay by Flow Cytometry and Caspase Activity

One million B16 cells were transfected with saline or 1 μg mRNA encoding luciferase, tBid or MLKL and at different time points the cells were analyzed. The cells were washed in Annexin V binding buffer (BD Biosciences, 556454), followed by a staining with Sytox Blue Nucleic Acid Stain (Molecular Probes, S11348) and APC Annexin V alexa fluor 488 conjugate (Molecular Probes, A13201). The experiments were performed on a triple-laser (B-V-R) LSR-II (Becton Dickinson, San Jose, Calif., USA) and analyzed using FlowJo software (Treestar, Oreg.). First single cells were selected based on their forward scatter (FSC) and side scatter (SSC). Next necroptotic and apoptotic cells were identified based on annexin-V and SYTOX blue positivity.

B16 cells (10⁶ cells/well in 6-well plate) were analyzed at different time points after transfection with saline or 1 μg of mRNA encoding Fluc, tBid or MLKL. The extent of membrane permeability was assessed by staining with Sytox Blue Nucleic Acid Stain (Molecular Probes, S11348). The cells were analyzed on a triple-laser (B-V-R) LSR-II (Becton Dickinson, San Jose, Calif., USA). First single cells were selected based on their forward scatter (FSC) and side scatter (SSC). Next dead cells were identified based on SYTOX blue positivity (FIG. 10: gating strategy). The flow cytometry data were analyzed with FlowJo (Treestar, Oreg.).

To analyze caspase activity and cell death a FLUOstar OMEGA (BM, labtech) assay was performed. Therefore, 5.10³ cells were seeded in a transparent 96-well plate and transfected with saline or 5 ng of mRNA encoding Fluc, tBid or MLKL 2 μM of SYTOX Green nucleic acid stain (Molecular Probes (S7020) and 33 mM of Ac-DEVD-AMC (Peptanova, 317-V) was added to the cells. Cell death was measured based on SYTOX Green fluorescence (excitation 485 nm, emission 520 nm) and set relative to the signal of 0.05% of triton X-100 treated cells. Caspase activity was measured by cleavage of Av-DEVD-AMC into fluorescent 7-amino-4-methylcoumarin (AMC) (excitation 355 nm, emission 460 nm). The DEVDase activity is expressed as fold induction compared to the maximal fluorescence intensity value of cells treated with 10 000 U/ml TNF (eBioscience) and 2 μM TAK inhibitor (Analyticon Discovery GmbH). To analyze MLKL protein expression and caspase 3 cleavage, a Western blot was performed. B16 cells (10⁶ cells/well in 6-well plate) were transfected with saline or 1 μg of mRNA encoding Fluc, tBid or MLKL. Twenty four hours after transfection, the lysates were separated by SDS-PAGE (10% acrylamide) and MLKL and caspase-3 were visualized by Western blotting with antibodies directed against MLKL and full length and cleaved caspase-3.

To analyze the possible induction of NF-Kb upon cell death evoked by the mRNAs a luciferase assay was performed. B16 cells were seeded at 5×10⁴ cells per well in 24-well plates 24 hours before transfection. Cells were transfected with 50 ng of a plasmid in which the coding sequence of luciferase is under the control of the NF-κb promoter and 100 ng of a plasmid expressing β-galactosidase. Twenty four hours later the B16 cells were transfected with saline or 100 ng of mRNA encoding GFP, tBid or MLKL or, as a positive control, the B16 cells were transfected with 25 ng TRAF6 expression plasmid or stimulated with 100 U/ml TNF. At different time points, cells were lysed with luciferase lysis buffer (25 mM Tris-phosphate, 2 mM DTT, 2 mM CDTA, 10% glycerol and 1% Triton X-100). Luciferase activity was measured with a GloMax® 96 Microplate Luminometer (Promega) by adding luciferin to the lysates. To normalize the luciferase activity, the B-galactosidase activity was measured on a iMark microplate reader (Biorad). The ratio of the β-galactosidase and luciferase activities was determined to normalize for transfection efficiency.

To investigate if the evoked cell death requires transcription a flow cytometry experiment was performed. All B16 cells (10⁶ cells/well in 6-well plate) were transfected with 1 μg of a GFP expressing plasmid and with saline or 1 μg of mRNA encoding Fluc, tBid or MLKL. Next to this transfection, in half of the condition actinomycin D (1 μg/ml) was added to the cell culture medium. Twenty four hours after transfection, the extent of membrane permeability was assessed by staining with Sytox Blue Nucleic Acid Stain (Molecular Probes, S11348). The cells were analyzed on a triple-laser (B-V-R) LSR-II (Becton Dickinson, San Jose, Calif., USA). First single cells were selected based on their forward scatter (FSC) and side scatter (SSC). Next dead cells and GFP expressing cells were identified based on SYTOX blue positivity (FIG. 10: gating strategy) of GFP positivity. The flow cytometry data were analyzed with FlowJo (Treestar, Oreg.).

A.1.5. Cell Death Assay on FLUOstar OMEGA

To analyze caspase activity and cell death a FLUOstar OMEGA (BM, labtech) assay was performed. Five thousand cells were seeded in a transparent 96-well plate 24 h before the analyses. Cells were transfected with saline or 1 μg mRNA encoding luciferase, tBid or MLKL. Two μM SYTOX Green nucleic acid stain (Molecular Probes (S7020) and 33 mM Ac-DEVD-AMC (Peptanova, 317-V) were added to the cells. Maximum cell death was obtained by treatment with Triton X-100 (0.05%). This allowed the expression of the cell death as a percent of the control of maximal SYTOX Green fluorescence (excitation 485 nm, emission 520 nm). If the executor caspase 3/7 are activated during cell death, they cleave Av-DEVD-AMC only upon plasma membrane rupture, resulting in the release of fluorescent 7-amino-4-methulcoumarin (AMC) (excitation 355 nm, emission 460 nm). The DEVD activity is expressed as fold induction compared to the maximal fluorescence intensity value.

A.1.6. Live Cell Imaging

Fifteen thousand cells were seeded per well of an eight-well chamber (iBidi) in 200 μl complete growth medium. Twenty four hours later, cells were transfected with saline or 1 μg mRNA encoding luciferase (Fluc), tBid or MLKL just before imaging. Live-cell imaging was performed on a Leica Sp5 AOBS confocal microscope (Leica), using 40×HOC PL Apo UV 1.25 na oil objective. Images were acquired in a sequential mode every 30 min.

A.1.7. Transfection In Vivo

C57BL/6 mice and Balb/cAnNCrI mice were shaved at the site of tumor growth. 10 μg mRNA dissolved in 10 μl Hank's Balanced Salt Solution (HBSS) (Gibco®) was injected in the tumor using a U-100 insulin needle (BD Biosciences, San Diego, Calif., USA). Next a conductive gel (EKO-GEL, ultrasound transmission gel, Egna, Italy) was applied at the tumor site to ensure electrical contact with the skin and electroporation was performed. Two pulses of 20 ms and 120 V/cm were delivered through spaced plate electrodes by a ECM® 830 Electroporation System (BTX® Harvard apparatus)

A.1.8. Mice

Female C57BL/6 mice were purchased from Charles River France. Female Balb/cAnNCrI mice were purchased from Charles River Italy (via France). OT-I mice carrying a transgenic CD8⁺ T cell receptor specific for the MHC-I restricted OVA peptide (257-264), OT-II mice carrying a transgenic CD4⁺ T cell receptor specific for the MHCII restricted OVA peptide (323-339), the CCR-7 deficient mice, the batf3 deficient mice and the Type I IFN deficient mice were bred at the breeding facility of the Vlaams Instituut voor Biotechnology (VIB, Ghent, Belgium). NSG mice were bred at the breeding facility of the university hospital Ghent (UZ Ghent, Belgium). All mice were 7-10 weeks old at the start of the experiment. Animals were housed under specific pathogen-free conditions in individually ventilated cages in a controlled 12 h day-night cycle and given food and water ad libitum. All procedures involving animals were approved by the local Ghent University ethics committee (accreditation nr. LA1400536, Belgium), in accordance with European guidelines. Mice experiments are covered under the following EC applications: EC2016-010 and ECD17/11.

A.1.9. Tumor Implantation and Tumor Growth Measurement

In total, 5×10⁵ B16 (OVA) cells or CT26 cells diluted in 100 μl HBSS were injected subcutaneously into the right flank of each C57BL/6 or Balb/cAnNCrI mice, respectively. At day 6 and 10 after inoculation of the tumor cells, the mRNA was injected in the tumor and the tumor was subsequently electroporated. For the comparison of the mRNA treatment with an antracyline treatment, B16 inoculated mice received doxorubicine (3 mg/kg) at d6, d8 and d10 or during three weeks every two days. Unless otherwise indicated, these doxorubicine treatments were done perilesionally, which is subcutaneously at the tumor border. For the combination therapy, 200 μg anti-PDL1 or an isotype control antibody was injected intraperitoneal during three weeks every three days.

Depending on the set-up of the experiment, the primary tumor was removed and 5×10⁵ B16 cells or CT26 cells diluted in 100 μl HBSS were injected subcutaneously into the left flank of each C57BL/6 or Balb/cAnNCrI mice respectively or 2×10 B16F10 or CT26 cells were injected i.v. The tumor size was measured every two days with an electronic digital caliper. Tumor volume was calculated as the length×width×height (in mm³). The mice were euthanized when the volume of the tumor reached 2000 mm³. For the experimental lung colonization assay experiments, mice were euthanized 12 days after i.v. injection of the tumor cells and tumor nodules were counted. In one experiment tBid and MLKL mRNA treated animals were sacrificed 22 days after i.v. injection of B16F10 cells. In the CT26 model, lung tumor burden was quantified after tracheal ink (1:10 diluted in PBS) injection and fixation with Fekete's solution (5 ml 70% ethanol, 0.5 ml formalin, and 0.25 ml glacial acetic acid).

A.1.10. In Vivo Bioluminescence Imaging

For in vivo imaging, mice were inoculated with 5×10⁵ B16 cells. Six days after inoculation 10 μg mRNA coding for luciferase was injected in the tumor. 150 mg/kg of D-luciferin (PerkinElmer, Waktham, Mass., USA) in PBS was injected i.v. at different time points and luciferase expression was monitored using an IVIS lumina II imaging system. Photon flux was quantified using the Living Image 4.4 software (all from Caliper life sciences, Hopkinton, Mass., USA).

A.1.11. Generation of Mouse BMDCs

BMDCs were differentiated from the femurs and tibias of 7-week-old C57BL/6 mice for 8 days using RPMI medium (Gibco), supplemented with 5% heat-inactivated fetal calf serum, L-glutamine (0.03%), sodium pyruvate (0.4 mM), 2-mercapthoethanol (50 mM) and mGM-CSF (20 ng/ml). Fresh culture medium was added on day 3 and on day 6 the medium was refreshed.

A.1.12. Analysis of Maturation of BMDCs Upon Co-Culturing

B16 cells were transfected with no mRNA, luciferase mRNA, tBid mRNA or MKL mRNA. Four hours later the cells were collected, washed and co-cultured with BMDCs in a 1:10 ratio for 24 h. Next the co-cultured cells were harvested, incubated with anti-CD16/CD32 (500× dilution) (BD Biosciences, San Diego, Calif., USA), immunostained with CD11c-PerCP-cy5.5 (200× dilution), MHCII-APC-cy7 (100× dilution), CD26-FITC (200× dilution), CSF1R-APC (200× dilution), (Invitrogen), CD-80-V450 (200× dilution), CD40-PE (400× dilution), CD86-PE-cy7 (400× dilution) (all BD Biosciences, San Diego, Calif., USA) and Fixable Viability Dye (1000× dilution). The experiments were performed on a four-laser Fortessa (Becton Dickinson, San Jose, Calif., USA) and analyzed using FlowJo software (Treestar, Oreg.). First live single cells were identified based on SSC, FSC and live dead stain. Macrophages and BMDCs were gated based on CD11C⁺ and MHCII⁺. Next BMDCs were identified on their CD26 expression and the lack of CSF1R expression.

A.1.13. Analysis of DC Infiltration in the Draining Lymph Nodes

Five hundred thousand B16 cells diluted in 100 μl HBSS were injected subcutaneously into the right flank of each C57BL/6 mouse. At day six and ten after inoculation of the tumor cells, mRNA was injected in the tumor and the tumor was subsequently electroporated. Two days after the second treatment, the draining lymph nodes were dissected and passed through 70 μm nylon strainers (BD Biosciences, San Diego, Calif., USA) to obtain single cell suspensions. Cells were stained with anti-CD16/CD32 (500× dilution) (BD Biosciences, San Diego, Calif., USA) to block Fc receptors followed by staining with Fixable Viability Dye efluor 780 (1000× dilution) (eBioscience), Ly6C-FITC (BD Biosciences), XCR1-BV510 (Biolegend), CD172a-PerCP-efluor710 (eBioscience), CD64-biotin BV786 SA (Biolegend), CD207-AF647 (eBioscience), CD11c-PE-efluor610 (eBioscience), MHCII-AF700 (eBioscience), CD3-PE-cy5 (eBioscience), CD19-Pe-cy5 (eBioscience), CD40-PE, CD86-Pe-cy7 and CD80-efluor450. The experiments were performed on a five-laser fortessa (Becton Dickinson, San Jose, Calif., USA) and analyzed using FlowJo software (Treestar, Oreg.). First live single cells were identified based on SSC, FSC and live/dead stain. T and B cells were gated out based on their CD3 and CD19 positivity respectively. Subsequently moDC were identified as CD64⁺MHCII⁺ cells, cDC1 as CD64⁻ MHCII⁺ CD11c⁺ XCR1⁺ and cDC2 as CD64⁻ MHCII⁺ CD11c⁺ CD127α⁺ cells. The activation status of cDC1s and cDC2s was analyzed based on the CD40, CD86 and CD80 expression level.

A.1.14. In Vivo T Cell Proliferation Assay

Two days before mRNA immunization, 2.10⁶ OT-I or OT-II cells were purified and labeled with 5 μM carbocyfluorescein diacetate succinimedyl ester (CFSE; Invitrogen, Merelbeke, Belgium). Two million CFSE-labelled OT-I or OT-II cells were i.v. injected into mice that had been s.c. inoculated with B16 cells two days before mRNA treatment. Four days after the mRNA treatment draining lymph nodes were isolated and OT-I or OT-II cell division was analyzed by flow cytometry. Cells were stained with anti-CD16/CD32 (500× dilution) (BD Biosciences, San Diego, Calif., USA) to block Fc receptors followed by staining with Fixable Viability Dye (1000× dilution) (eBioscience), anti-CD8 Pe-cy7 (eBioscience), anti-CD3 efluor450 (eBioscience), anti-CD19 APC (BD Biosciences) (all 200× dilution) and MHC-I dextramer H-2 Kb/SINFEKL-PE (10× dilution) (immundex, Copenhagen, Denmark). The experiments were performed on a triple-laser (B-V-R) LSR-II (Becton Dickinson, San Jose, Calif., USA) and analyzed using FlowJo software (Treestar, Oreg.). Single cells are gated based on FSC and SSC. Living cells are selected and gated for CD3⁺ CD19⁻ T cells. Within CD8⁺ T cells or CD4⁺ T cells, OVA-specificity is gated by labeling with MHC-I SINFEKL-PE dextramer.

A.1.15. In Vivo Killing Assay

Splenocytes from female mice were pulsed with 1 μg/ml of the MHC-I restricted OVA₂₅₇₋₂₆₄ peptide or HIV-1 gag peptide as a control before labeling with 5 μM or 0.5 μM CFSE (Invitrogen, Merelbeke, Belgium) respectively. Labelled cells were mixed at a 1:1 ratio and a total of 1.5×10⁷ mixed cells were adoptively transferred into immunized mice three days after boost (second mRNA treatment). Splenocytes from mice were isolated 48 hrs later and passed through 70 μm nylon strainers (BD Biosciences, San Diego, Calif., USA) to obtain single cell suspensions. Red blood cells were lysed using ACK red blood cell lysis buffer (BioWhittaker, Wakersville, Md., USA). Next the splenocytes were analyzed on flow cytometry. Percentage antigen-specific killing was determined using the following formula: (1−(% CFSEₕᵢ cells/% CFSE^{low} cells)^{immunized mice}/(% CFSE^{hi} cells/% CSFE^{low} cells)^{non-immunized mice})×100. The experiments were performed on a triple-laser (B-V-R) LSR-II (Becton Dickinson, San Jose, Calif., USA) and analyzed using FlowJo software (Treestar, Oreg.). Single cells were gated based on their SSC and FSC. Next CFSE positive cells were selected.

A.1.16. Elispot

C57BL/6 mice were inoculated with 5×10⁵ B16 cells and at day six and ten mice were treated with saline or 10 μg mRNA encoding luciferase, tBid or MLKL. Two days after the second treatment, spleens were isolated and passed through 70 μm nylon strainers (BD Biosciences, San Diego, Calif., USA) to obtain single cell suspensions. Red blood cells were lysed using ACK red blood cell lysis buffer (BioWhittaker, Wakersville, Md., USA) and 2.5×10⁵ cells were cultured for 24 hours on IFN-γ (Diaclone, Besancon, France) pre-coated 96-well plates in the presence of 10 μg/ml peptide.

The following synthetic, HPLC-purified peptides were used for restimulation: OVA 257-264 (SIINFEKL; SEQ ID NO:3), OVA 323-339 (SQAVHAAHAEINEAGR; SEQ ID NO:4), CT26-M20 (PLLPFYPPDEALEIGLELNSSALPPTE; SEQ ID NO:5), CT26-M26 (VILPQAPSGPSYATYLQPAQAQMLTPP; SEQ ID NO:6), CT26-M03 (DKPLRRNNSYTSYIMAICGMPLDSFRA; SEQ ID NO:7), CT26-M37 (EVIQTSKYYMRDVIAIESAWLLELAPH; SEQ ID NO:8), CT26-M27 (EHIHRAGGLFVADAQVGFGRIGKHFW; SEQ ID NO:9), B16-M30 mut (PSKPSFQEFVDWENVSPELNSTDQPFL; SEQ ID NO:10), B16-M30 WT (PSKPSFQEFVDWEKVSPELNSTDQPFL; SEQ ID NO:11).

A.1.17. CD8 and CD4 Depletion

In total, 5×10⁵ B16 cells diluted in 100 μl HBSS were injected subcutaneously into the right flank of each C57BL/6 mice. At day 6 and 10 after inoculation of the tumor cells, the mRNA was injected in the tumor and the tumor was subsequently electroporated. In the CD8+ depletion assay, 200 μg anti-mouse CD8α antibody (clone YTS 169.4, BioXCell) was i.p. injected at day five and ten. In the CD4+ depletion assay, 200 μg anti-mouse CD4 antibody (done GK1.5, BioXCell) was i.p. injected at day three, six and nine. The tumor size was measured every two days with an electronic digital caliper. Tumor volume was calculated as the length×width×height (in mm³). The mice were euthanized when the volume of the tumor reached 2000 mm³.

A.1.18. In Vivo mRNA Electroporation

C57BL/6 mice and BALB/cAnNCrI mice that had been inoculated s.c. with tumor cells, were shaved at the site of tumor growth. 10 μg of mRNA dissolved in 10 μl of Hank's Balanced Salt Solution (HBSS; Gibco) was injected in the tumor using a U-100 insulin needle (BD Biosciences, San Diego, Calif., USA). Next a conductive gel (EKO-GEL, ultrasound transmission gel, Egna, Italy) was applied at the tumor site to ensure electrical contact of the electrodes with the skin and electroporation was performed. Two pulses of 20 ms and 120 V/cm were delivered through spaced plate electrodes by a ECM® 830 Electroporation System (BTX® Harvard apparatus). Mice were treated this way on day six and ten after tumor cell inoculation.

A.1.19. Mice with a Humanized Immune System

To obtain hematopoietic stem cells (HSC) that HLA type matched with the human RL tumor cells used for the antitumor experiment, cord blood cells were stained with HLA-A2-FITC (BD Pharmingen) or with HLA-ABC-PE (BD Pharmingen) as a positive control prior to HSC isolation. Samples were acquired on an Attune Nxt Acoustic Focusing Cytometer (Life Technologies). HLAA2⁺ samples were selected for CD34⁺ stem cells. To that end, viable mononuclear cells were isolated by gradient separation to isolate the viable mononuclear cells. Next, CD34⁺ cells were isolated using a direct CD34⁺ progenitor cell isolation kit (Miltenyi). To evaluate the purity of the isolated stem cells a flow cytometric staining was performed (human CD3-PE (BD Pharmingen)/human-CD34-APC (BD Pharmingen)). Samples were acquired on an Attune Nxt Acoustic Focusing Cytometer (Life Technologies). The purity of the injected cells reached 92-98%. To generate mice with a humanized immune system, newborn NSG mice (2 days old) received a sublethal irradiation of 100 cGy followed by an intrahepatic delivery of CD34⁺ stem cells. Eight weeks and 12 weeks after CD34⁺ stem cell transfer, peripheral blood was analyzed for the presence of both human and mouse CD45⁺ (both BD) cells to analyze the effect of the engraftment. Samples were acquired on a LSR flow cytometer (BD) and analyzed by FACS Diva software (BD). The mice were s.c. inoculated with 2.5×10⁶ human RL follicular lymphoma cells at 13 weeks after stem cell transfer. From day 8 onwards, mice received on a daily base 30 μg FLT3L protein intraperitoneally. On days 11 and 15 after RL cell injection, the tumors were injected with saline or with 10 μg mRNA encoding Fluc or human MLKL followed by electroporation. Tumor growth was measured over time. The animals were euthanized when the tumor had reached a size of 1000 mm².

A.1.20. Hematologic Analysis

On day 11, 18 and 25 blood was collected from the tail vein in EDTA-coated microvette tubes (Sarstedt), and analyzed in a Hemavet 950FS (Drew Scientific) whole blood counter.

A.2. Results

A.2.1. mRNA Encoding MLKL Induces Necroptotic-Like Cell Death in Tumor Cells In Vitro and In Vivo

In vitro transcribed mRNA was used to express MLKL or control genes of interest because gene delivery by mRNA is safe and efficient. Moreover, in vitro transcription of capped and polyadenylated mRNA is a scalable process that can be made fully compliant with Good Manufacturing Practices (Grunwitz et al. 2017, Curr Top Microbiol Immunol 405:145-164; Diken et al. 2017, Curr Issues Mol Biol 22:113-128). Hypo-inflammatory mRNA was produced by replacing cytidine and uridine by 5-methylcytidine and pseudouridine, respectively. The transcripts contained a 5′ cap and 3′ poly(A) tail and the encoded open reading frame of interest was flanked by stabilizing 5′ and 3′ untranslated regions (FIGS. 8A and 8B).

Fluorescently labeled mRNA coding for GFP was rapidly taken up and translated following transfection of B16 melanoma cells in vitro (FIG. 8C). In vivo intra-tumoral mRNA delivery was performed by electroporation. Intra-tumor delivery of mRNA encoding luciferase resulted in a peak of reporter gene expression 12 h after electroporation (FIG. 8D). Next, mRNAs were designed that code for key signaling proteins of cell death, namely MLKL and tBid (truncated Bcl2-like inducer of cell death). MLKL is crucial for the execution of necroptosis while tBid, the caspase-cleaved form of Bid, is an inducer of intrinsic apoptotic cell death (Murphy et al. 2013, Immunity 39:443-453; Li et al. 1998, Cell 94:491-501; Letai et al. 2002, Cancer Cell 2:183-192). Transfection of B16 melanoma cells with mRNA encoding MLKL or tBid resulted in cell death but only tBid mRNA transfection gave rise to cells that became annexin V positive, which is a hallmark of apoptosis (FIGS. 9A & 15A). Transfection with tBid mRNA resulted in caspase activity and caspase-3 cleavage/maturation, and in cell death that could be prevented by adding the pan-caspase inhibitor zVAD-fmk (FIG. 9B). In contrast, caspase activity and caspase-3 cleavage/maturation were not detectable in MLKL mRNA transfected B16 melanoma cells and cell death was not affected by zVAD-fmk (FIGS. 9B & 15A). Cell death induced by mRNA encoding tBid or MLKL did not activate NF-Kb signaling (FIG. 15B). Moreover, the extent of cell death following tBid or MLKL mRNA transfection was comparable in the presence or absence of actinomycin D, suggesting that de novo transcription was not required (FIG. 15C).

Time-lapse microscopy was performed to visualize the morphology over time of the cell death progression in vitro of B16 cells after transfection with mRNA coding for MLKL or tBid. This imaging method revealed rounding up followed by swelling of the cells and eventually plasma membrane permeabilization of MLKL mRNA transfected cells, all of which are hallmarks of necroptotic cell death (Majno et al. 1995, Am J Pathol 146:3-15). Cells that had been transfected with tBid mRNA rounded up and showed membrane blebbing, which is a characteristic feature of apoptotic cell death (FIG. 9C). Finally, it was also evaluated if in vivo electroporation of intra-tumor injected MLKL and tBid encoding mRNA would result in cell death. Flow cytometry analysis of tumor cells that were isolated 24 h after mRNA transfection, revealed that MLKL mRNA electroporation resulted in cell death without annexin V exposure whereas tBid mRNA electroporation of tumor cells was associated with dying cells that became annexin V positive (FIG. 9D). MLKL- and tBid-mRNA electroporation resulted in a very similar level of cell death, which was approximately 3 fold higher than the extent of tumor cell death in the saline and irrelevant control mRNA settings (FIG. 9D).

Taken together these results show that it is possible to induce cell death in B16 cells with in vitro transcribed hypo-inflammatory mRNA coding for MLKL or tBid. The induced cell death shows necroptotic features in the case of MLKL transfection and apoptotic features in the case of tBid transfection.

A.2.2. Intra-Tumoral Delivery of MLKL Encoding mRNA Stalls Primary Tumor Growth and Protects Against Tumor Rechallenge and Metastasis

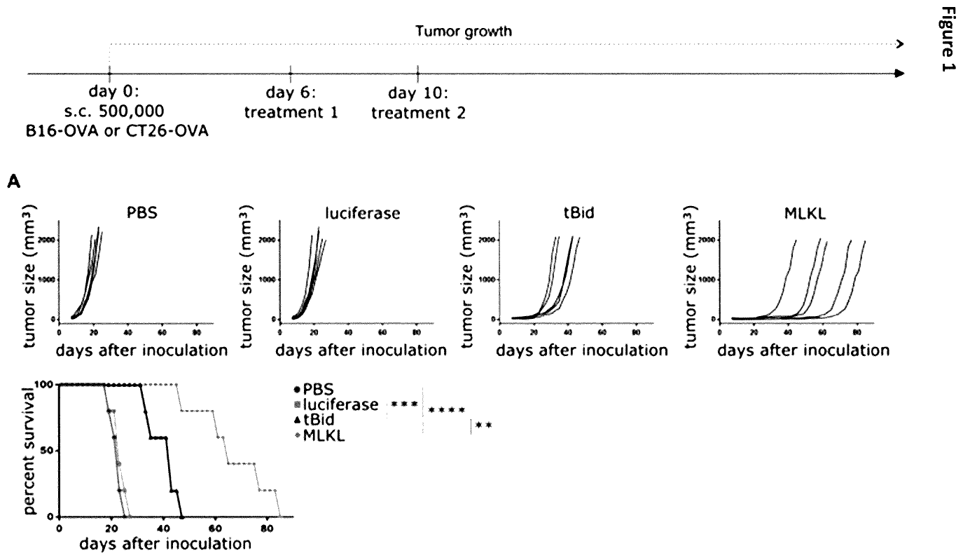

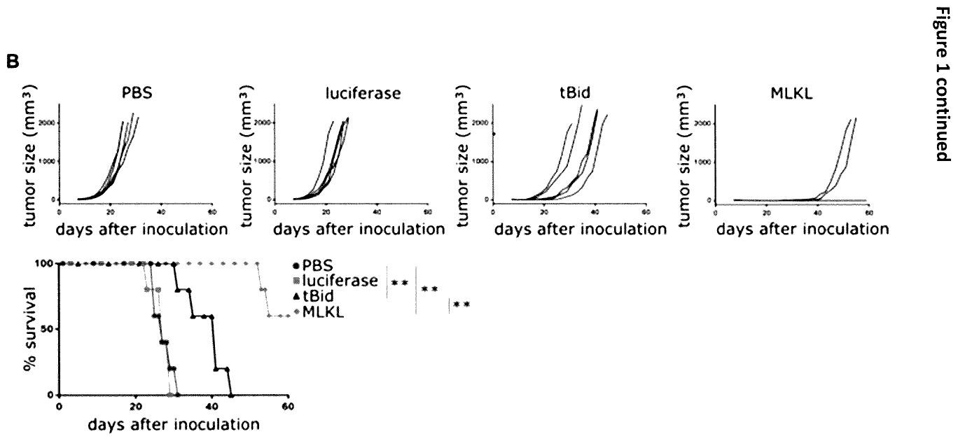

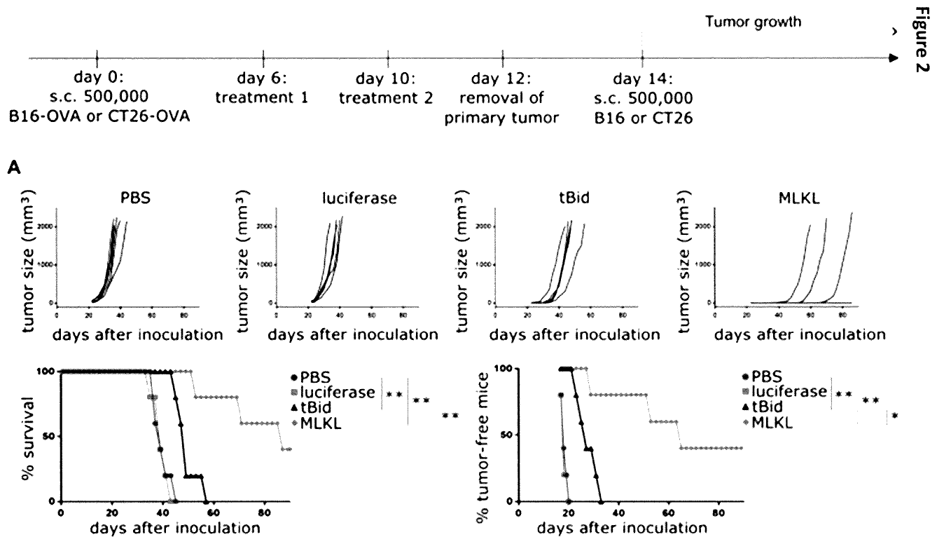

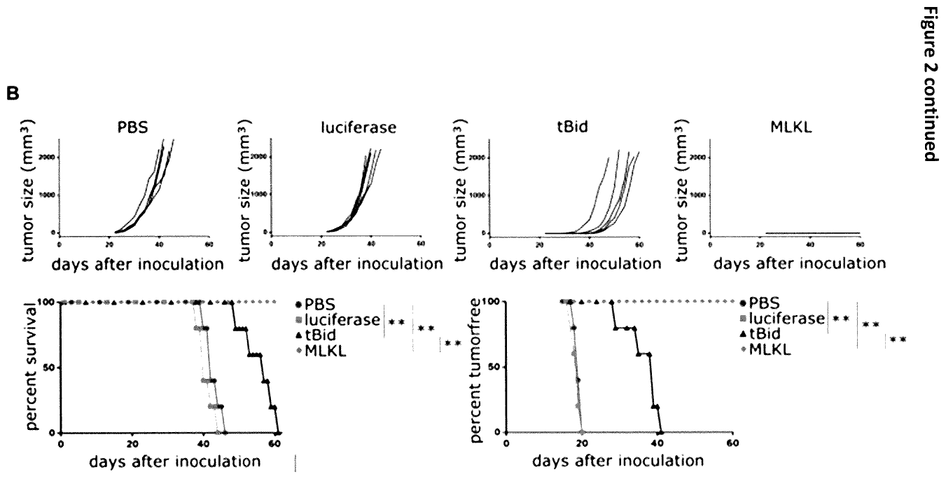

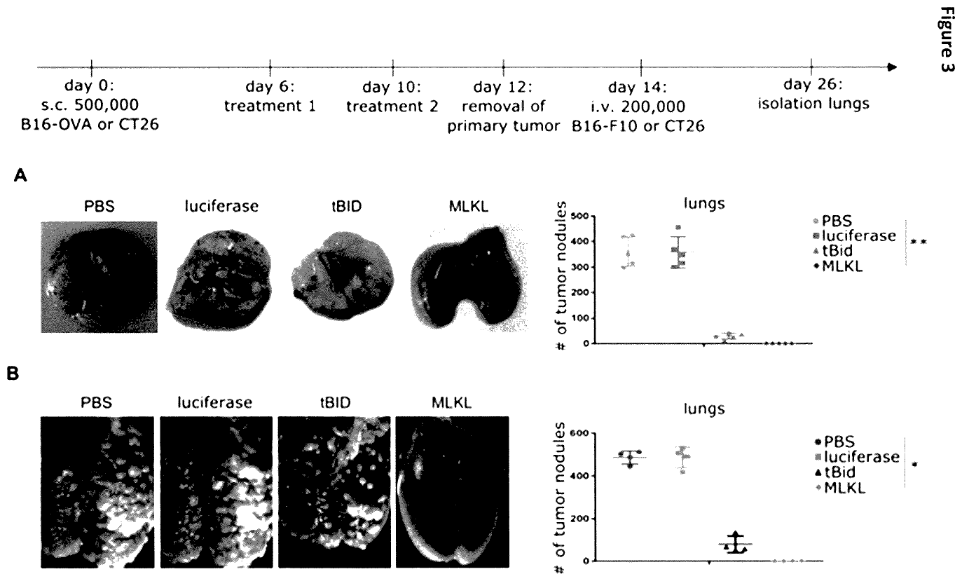

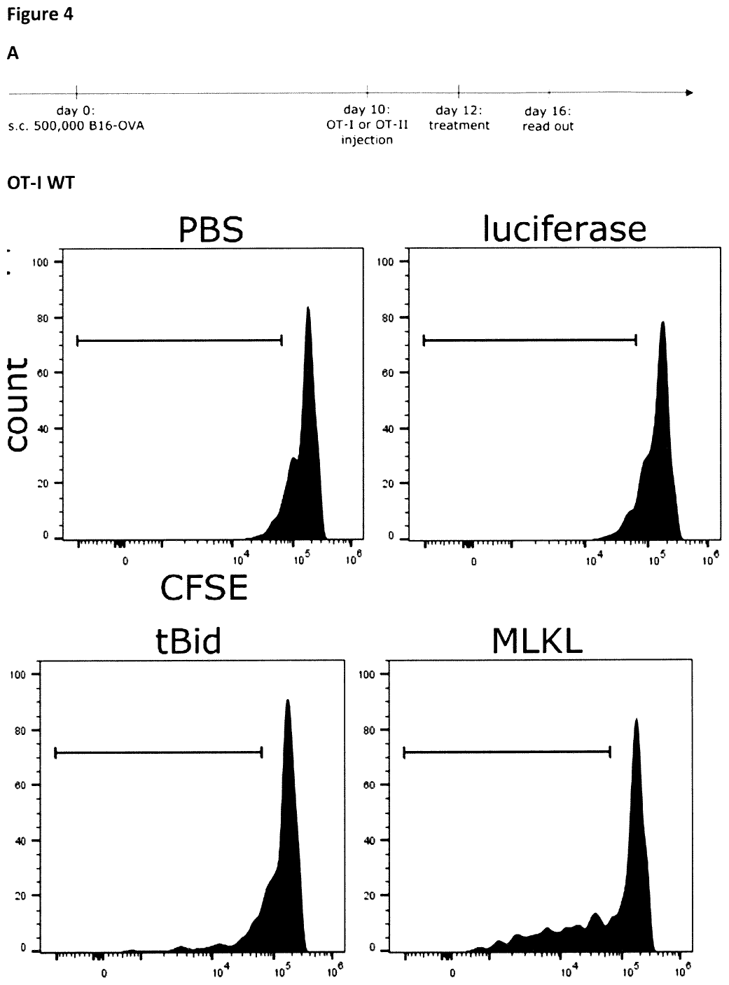

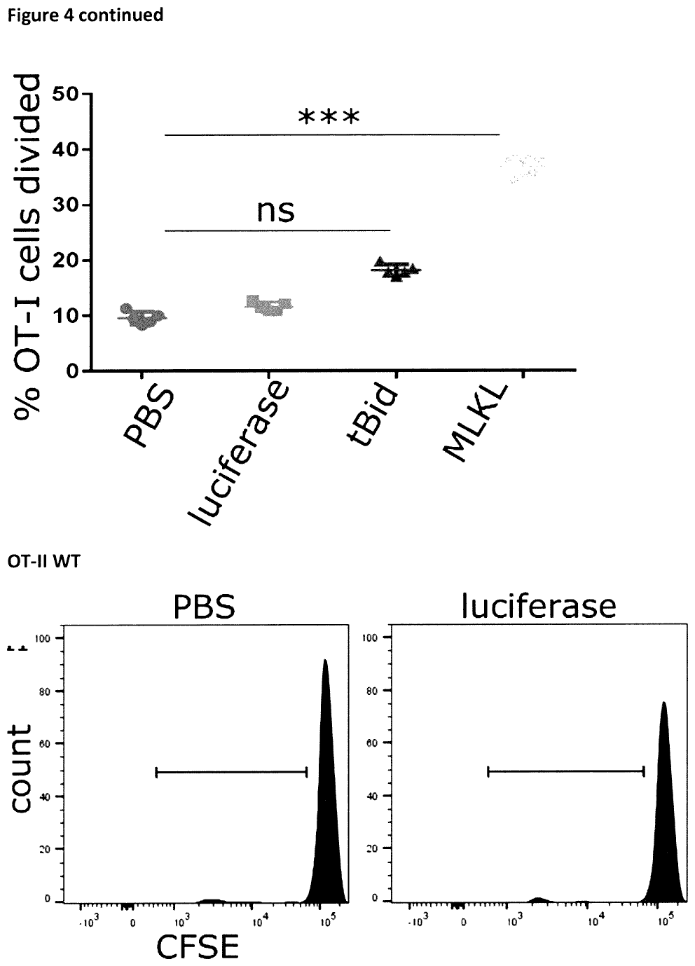

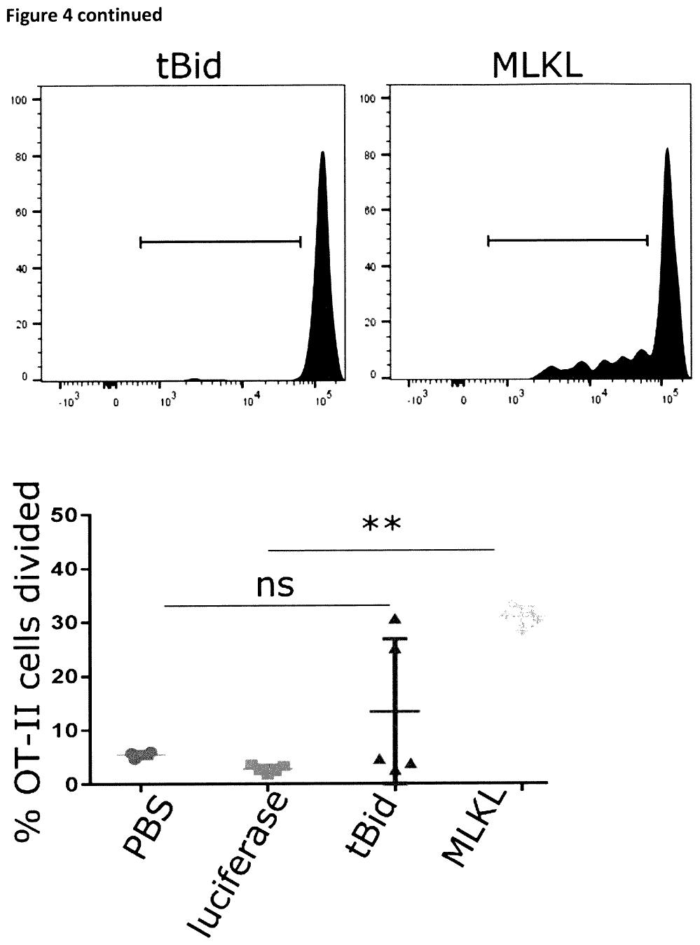

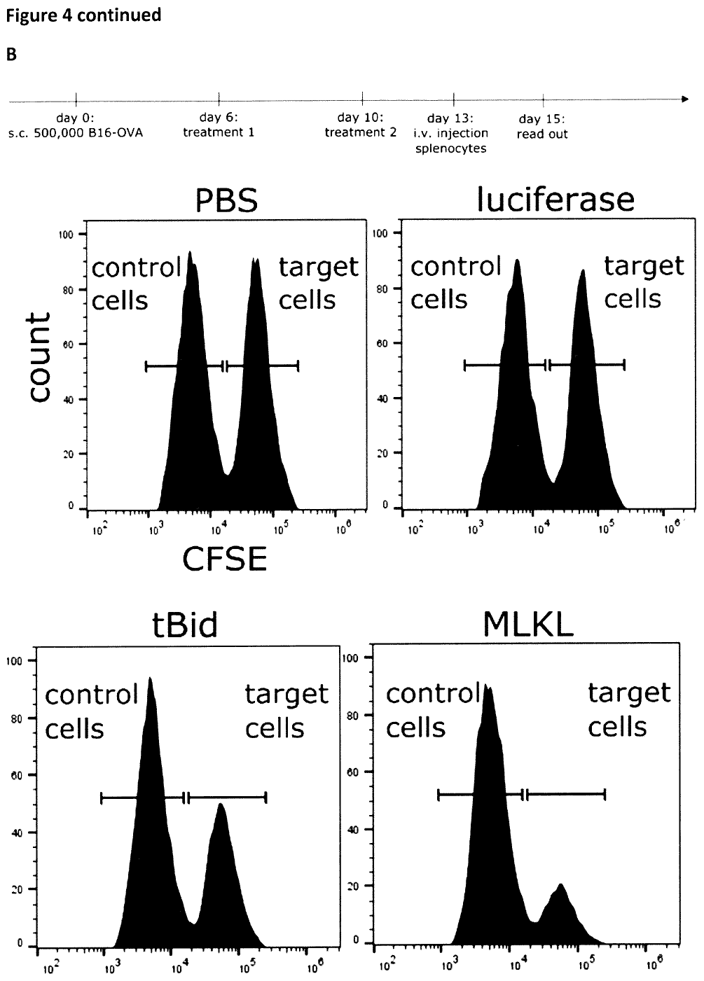

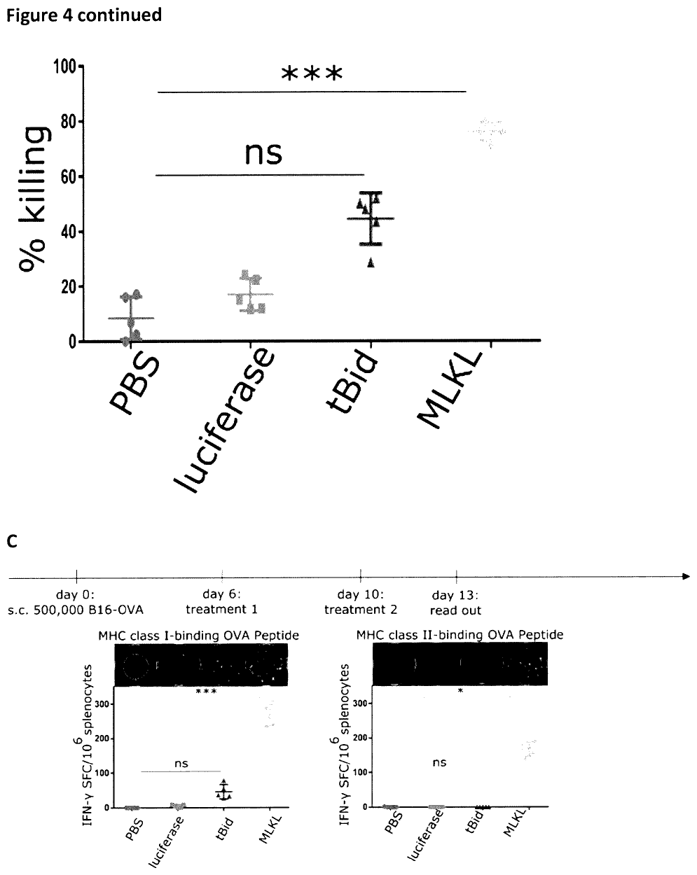

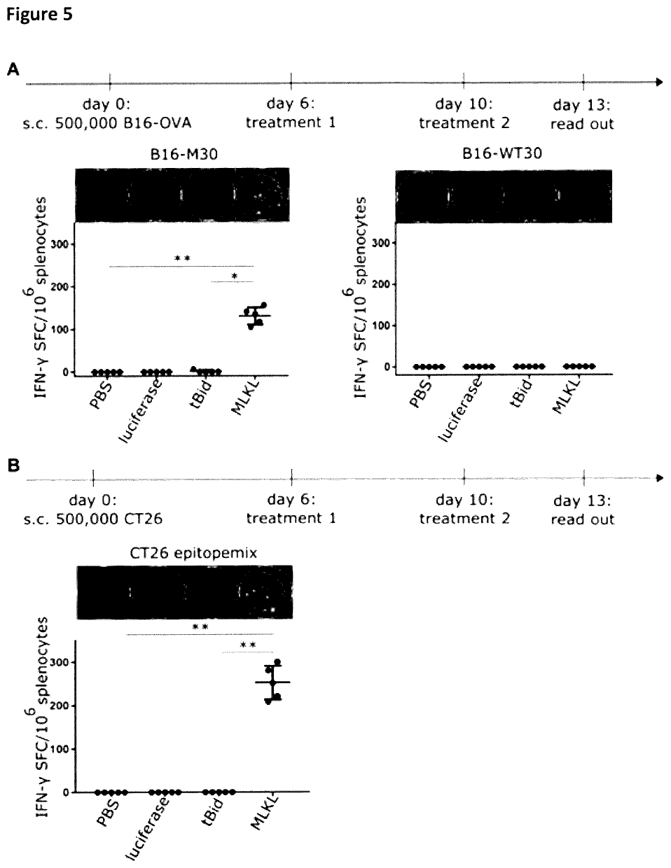

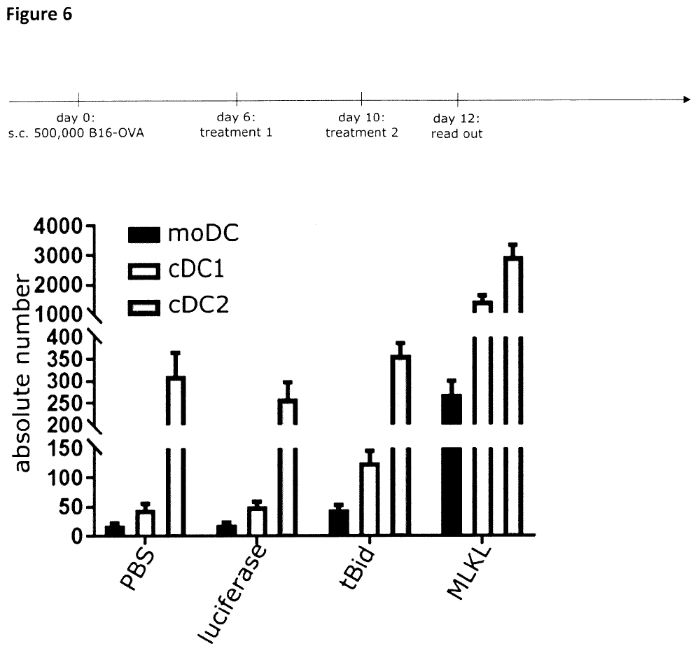

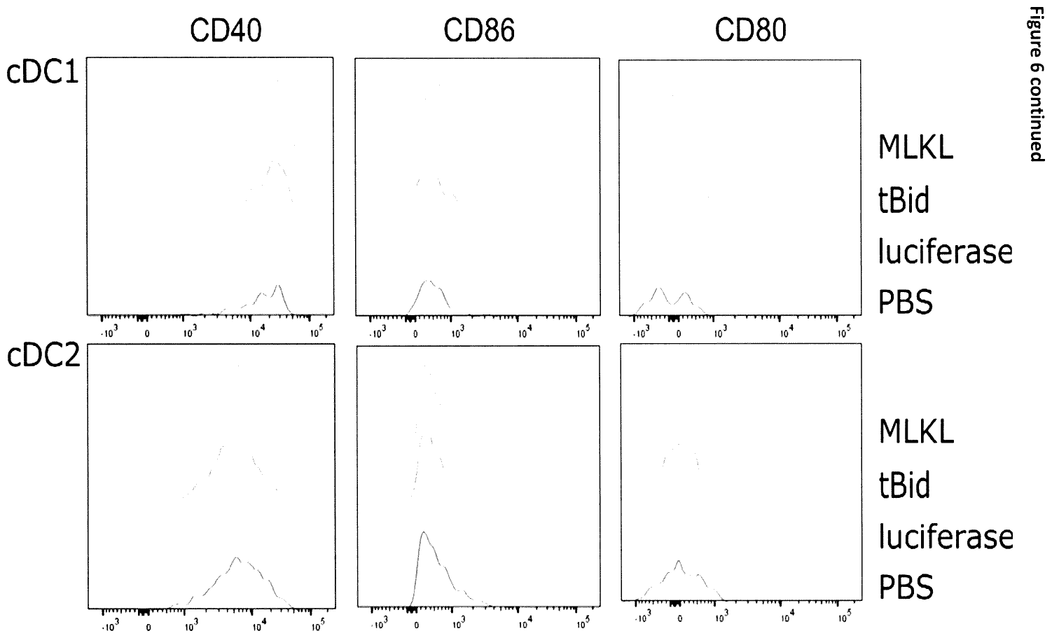

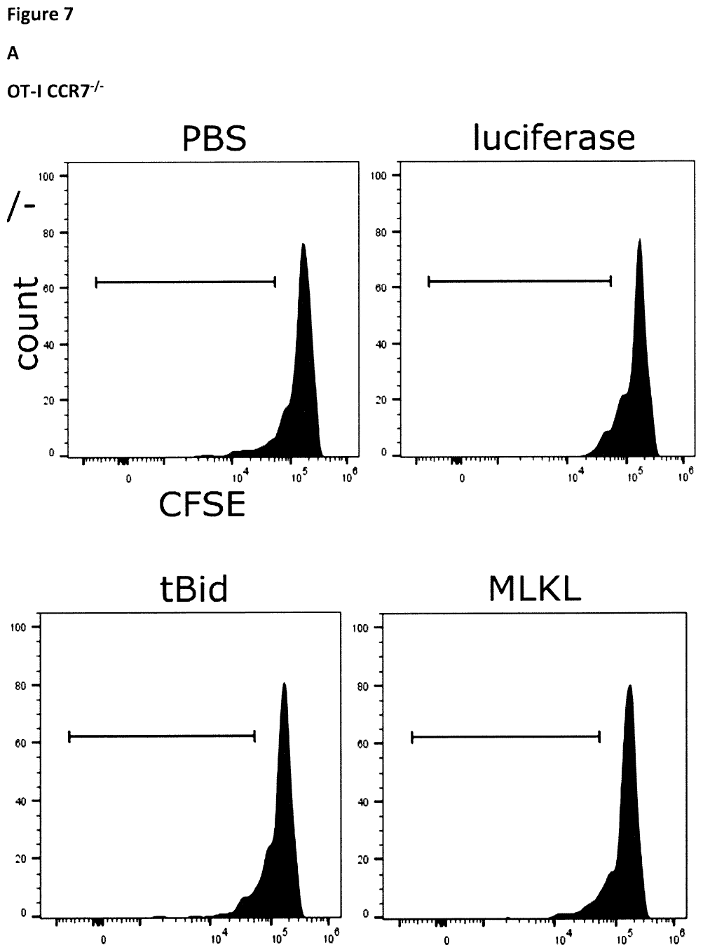

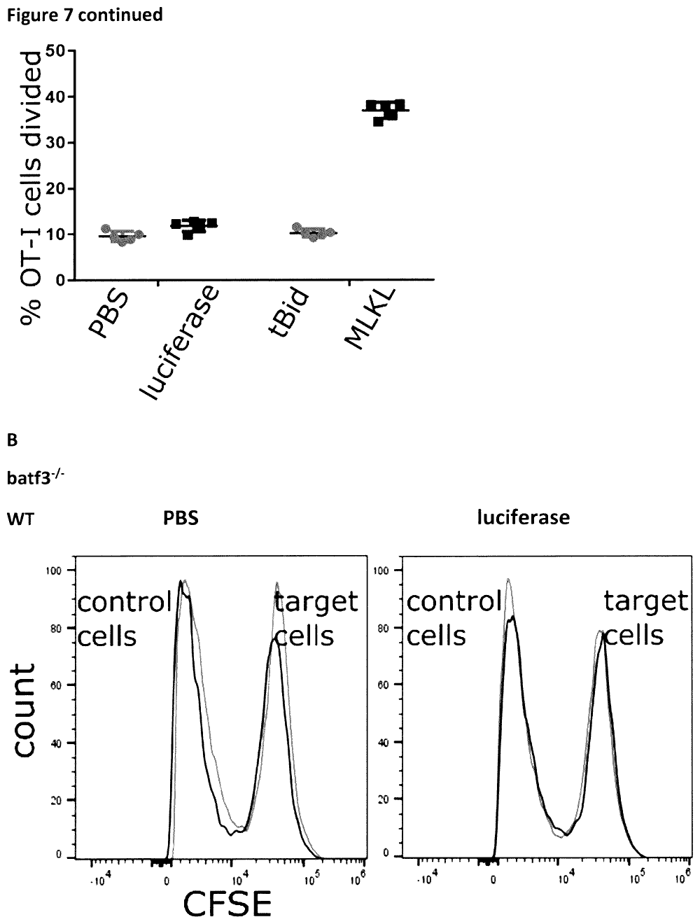

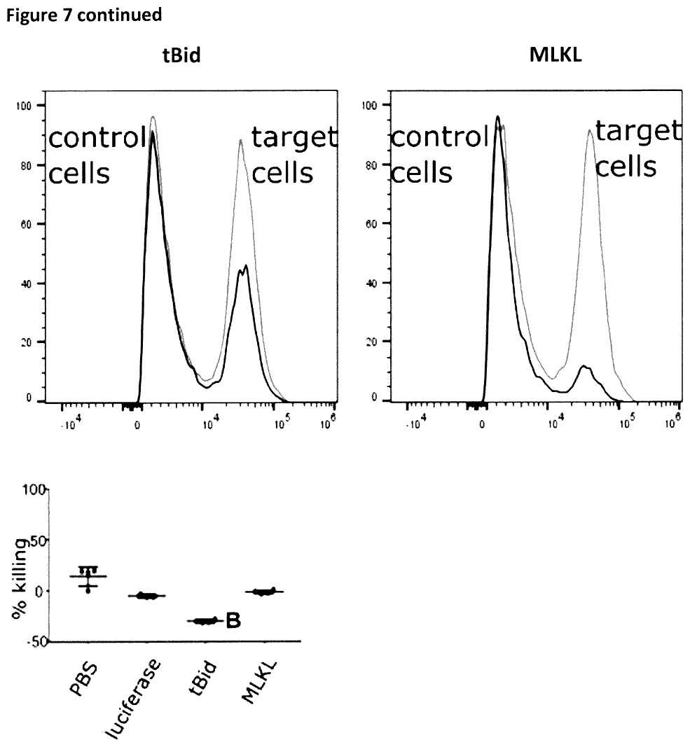

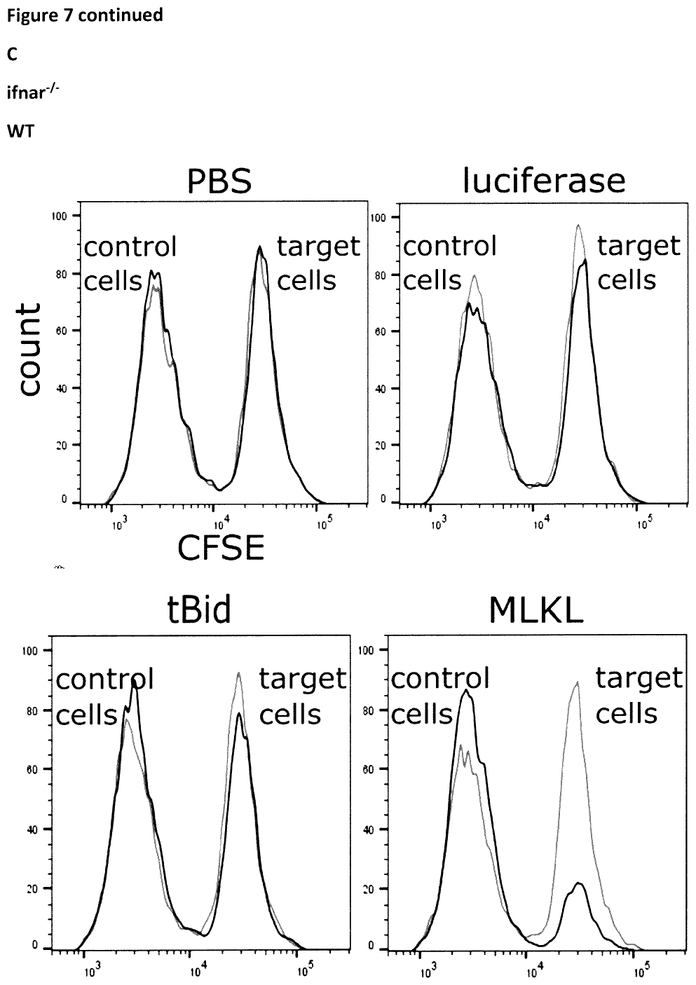

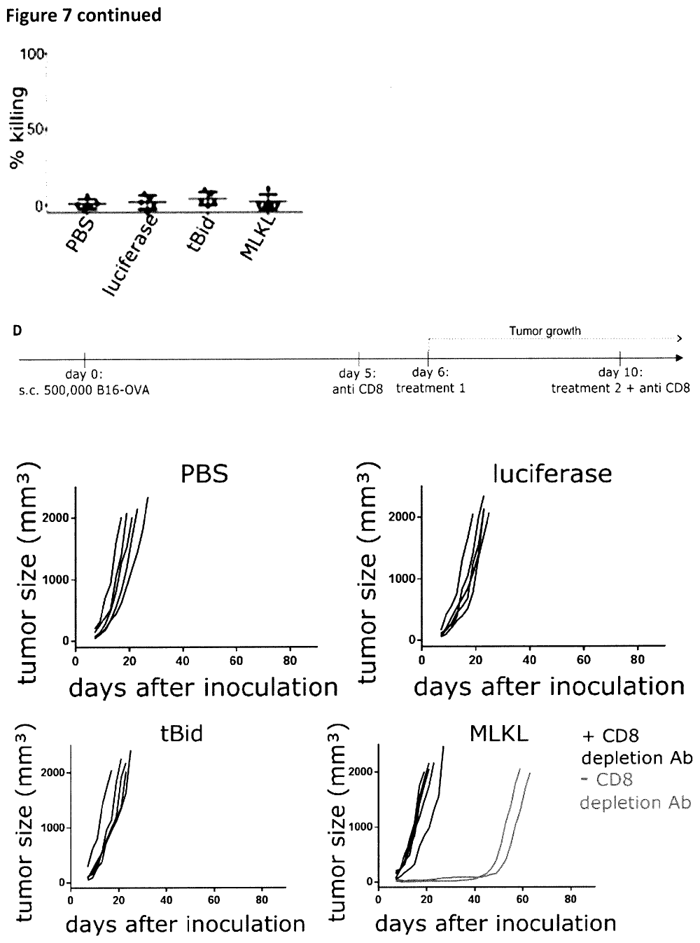

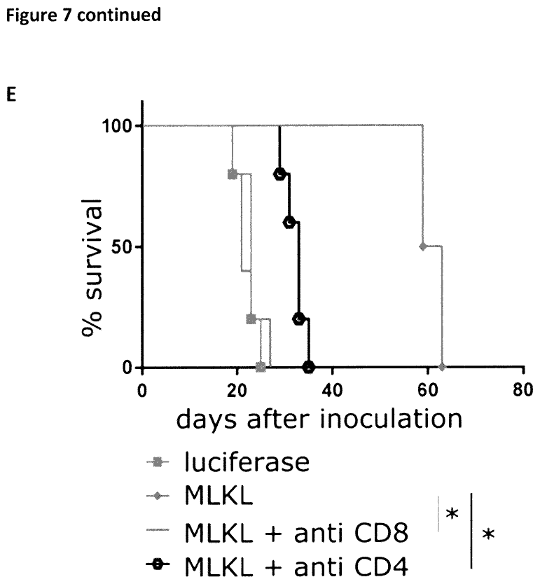

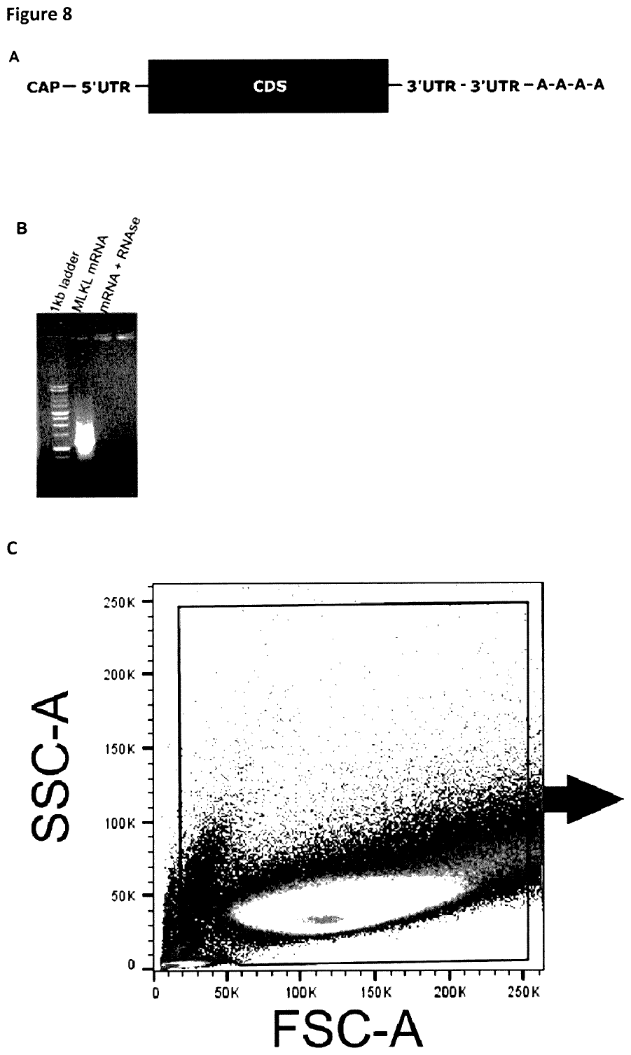

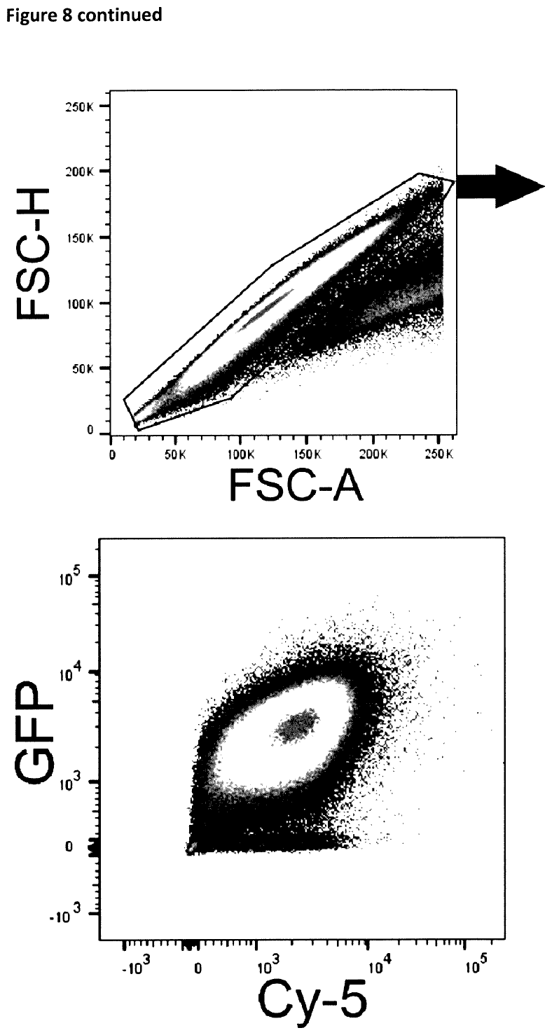

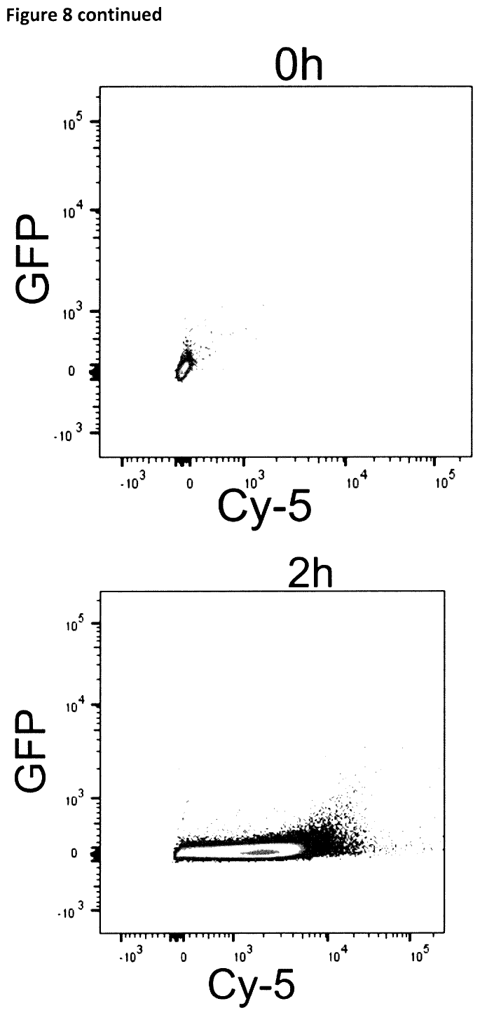

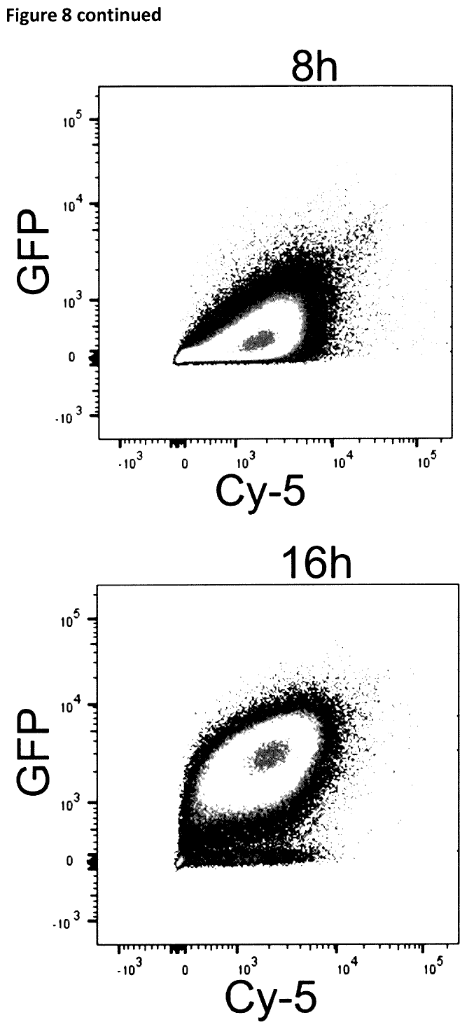

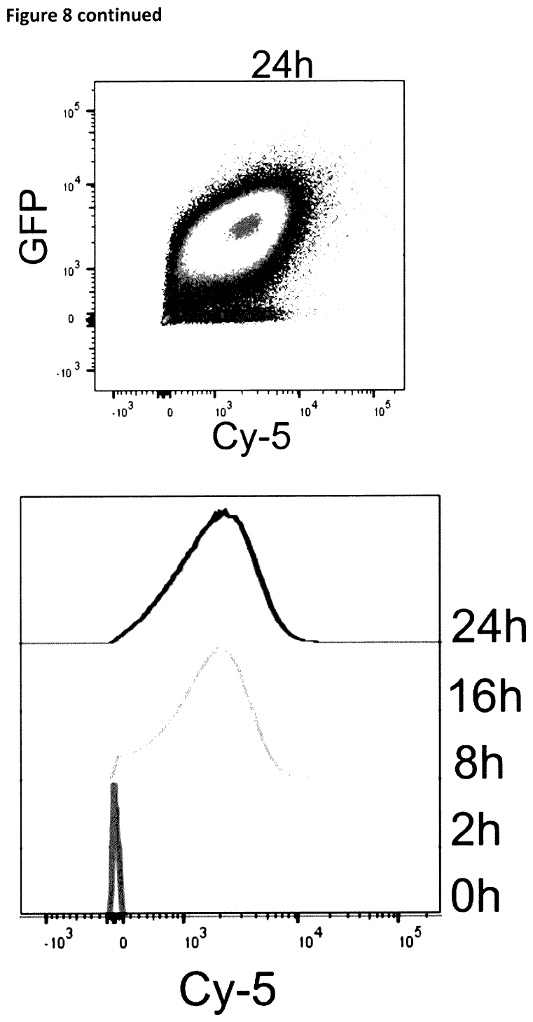

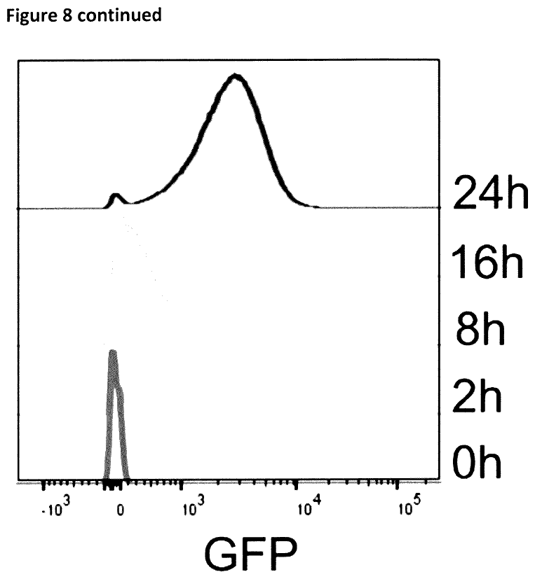

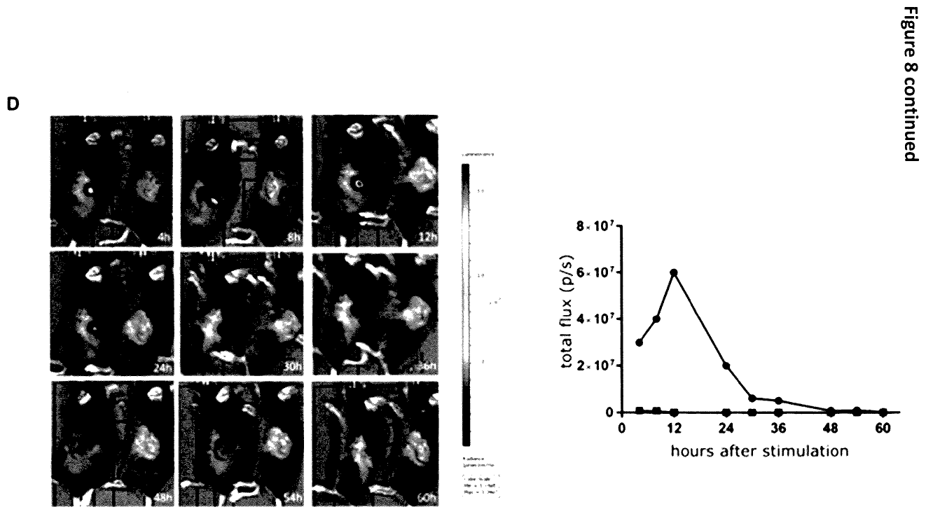

In a next step, it was evaluated in a mouse model if the intra-tumoral (IT) delivery if mRNA encoding tBid or MLKL could suppress the progression of primary tumor growth and so improve the survival. The mRNA-based treatments were tested in the syngeneic B16 (melanoma; an aggressive tumor model) (Griswold 1972, Cancer Chemother Rep Part 2, 3:315-324) and CT26 (colon carcinoma) (Brattain et al. 1980, Cancer Res 40:2142:2146) tumor models. For the B16 model, C57BL/6 mice were subcutaneously inoculated with 500,000 B16-ovalbumin (B16-OVA) melanoma cells. Six and 10 days later, the tumors were injected with saline or mRNA encoding luciferase, tBid or MLKL and subsequently in vivo electroporated to enable intracellular mRNA delivery. Mice were euthanized when the tumor had reached a size of 2,000 mm³. Tumor growth and time until the ethical endpoint was reached were comparable after IT-administration of saline or mRNA encoding luciferase (FIG. 1A). The tumor growth rate following saline injection and luciferase-mRNA injection followed by electroporation was identical, indicating that mRNA electroporation by itself does not induce adequate immune activation. In contrast, IT-treatment with tBid mRNA significantly delayed tumor growth and increased survival of mice from a median survival time of 23 days to 43 days (FIG. 1A). But the most striking anti-tumor effect was observed with mRNA encoding MLKL. This treatment resulted in a significant delay in tumor growth and significantly increased the median survival time (64 days) compared to the three other groups (FIG. 1A). Since, as noted above (A.2.1), tBid- and MLKL-mRNA treatment induced very similar levels of tumor cell death, we can also conclude that the mere induction of cell death is not sufficient to induce a strong anti-tumor response. Genetic and epigenetic alterations in the pathways that lead to necroptosis are common in cancer cells (Moriwaki et al. 2015, Cell Death Dis 6:e1636). Although CT26 tumor cells do not express RIPK3 (Aaes et al. 2016, Cell Rep 15:274-287), the therapeutic effect of MLKL-mRNA treatment resulted in a very pronounced antitumor effect.

The superior therapeutic potential of MLKL over tBid mRNA treatment was also observed in BALB/c mice that had been inoculated subcutaneously with the colon carcinoma cell line CT26. IT injection of mRNA encoding tBid followed by electroporation significantly delayed tumor growth and extended the survival time from 27 days to 41 days compared to saline or luciferase control mRNA treated mice (FIG. 1B). But again, the MLKL mRNA treatment resulted in the most pronounced anti-tumor effect, increasing the median survival time to more than 60 days and even 60% of the treated mice remained tumor free up to 80 days after CT26 inoculation (FIG. 1B).

Some of the standard of care therapies for cancer patients induce immunogenic cell death, such as treatment with doxorubicin (dox) (Obeid et al. 2007, Nature Med 13:54-61). Therefore, the anti-tumor effect of the MLKL mRNA treatment approach was compared with repeated injections of dox in the B16 melanoma model (FIG. 16A). Dox injections were administered every other day into the tumor or intraperitoneally for 3 weeks or mice from one group were treated on day 6, 8 and 10 only, by intra-tumor injection of dox. MLKL mRNA treatment was associated with significantly prolonged survival of the mice compared with any of the dox treatment set ups (FIGS. 16B and 16C). In addition, the prolonged treatment with dox during 3 weeks was associated with significantly reduced body weight loss and lymphocytopenia compared with the MLKL mRNA treated group (FIGS. 16D and 16E).

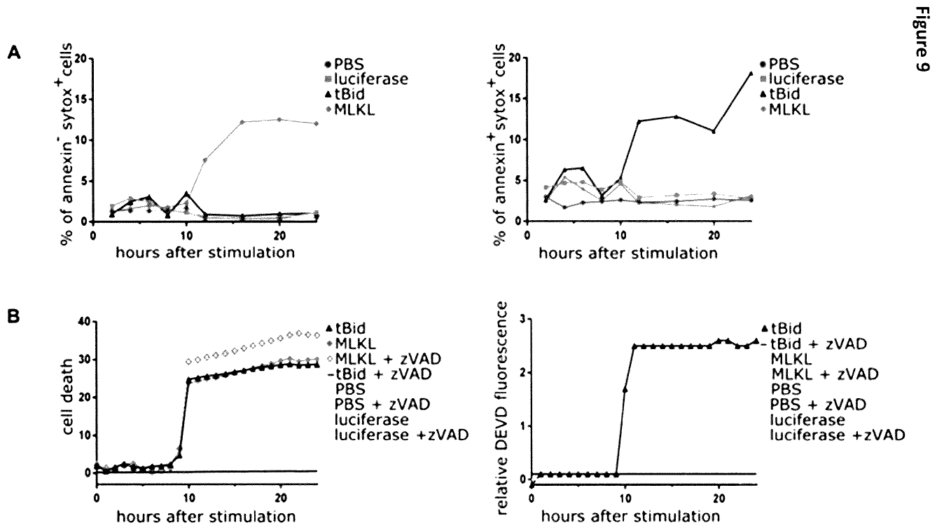

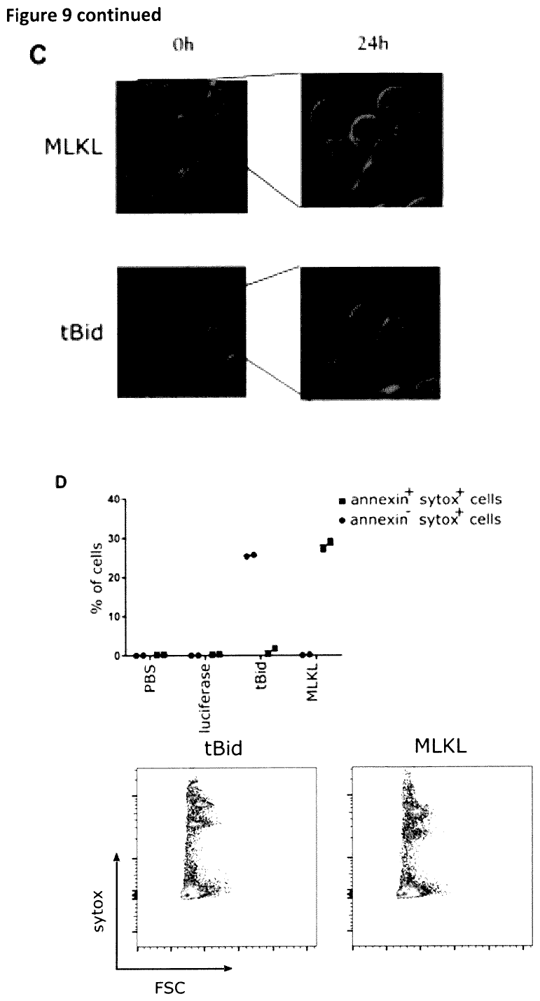

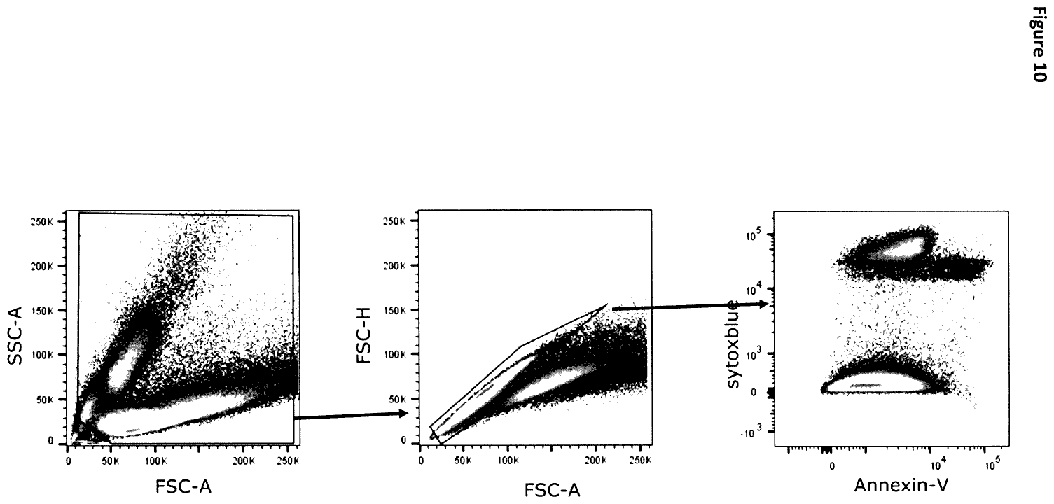

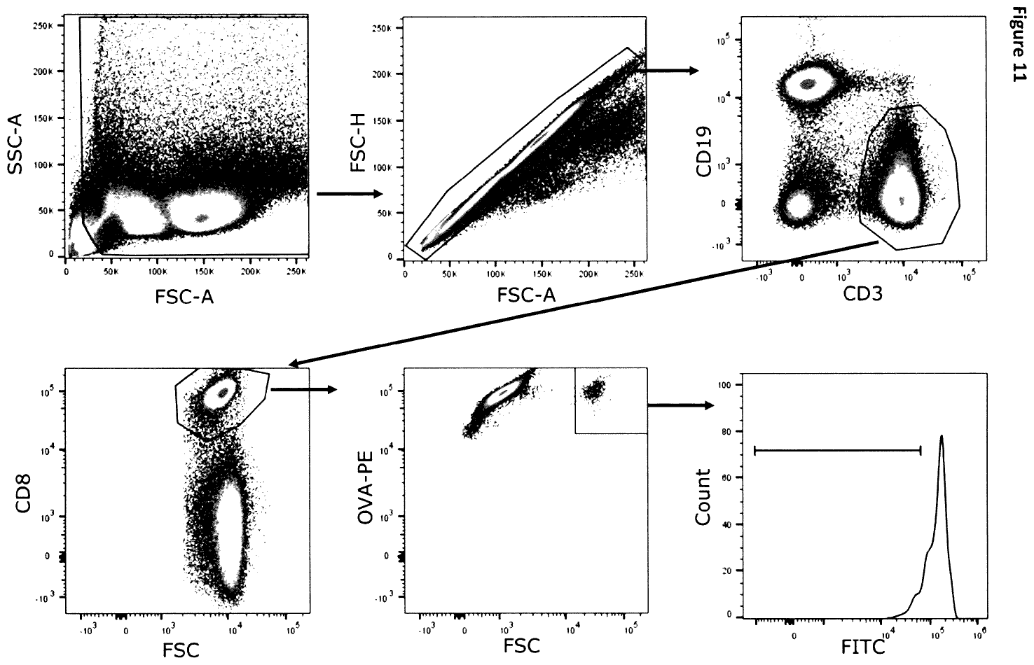

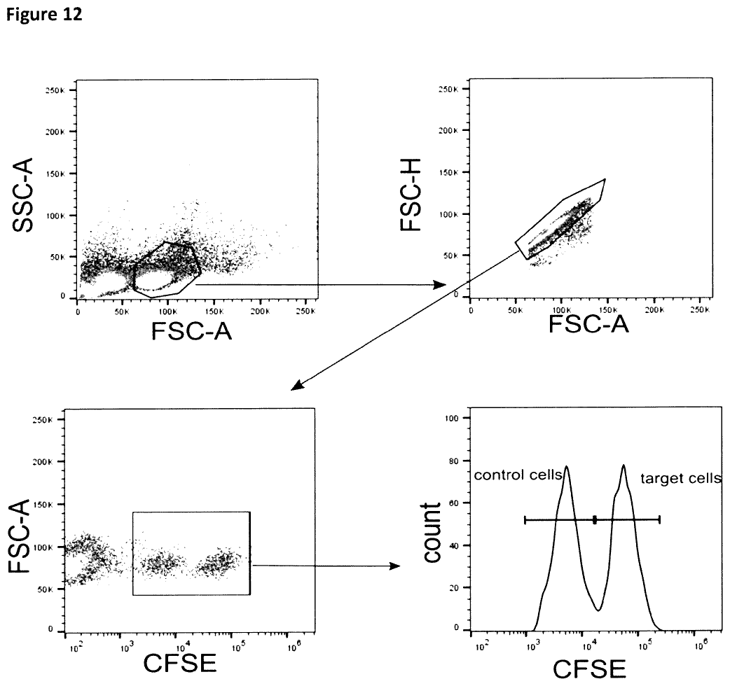

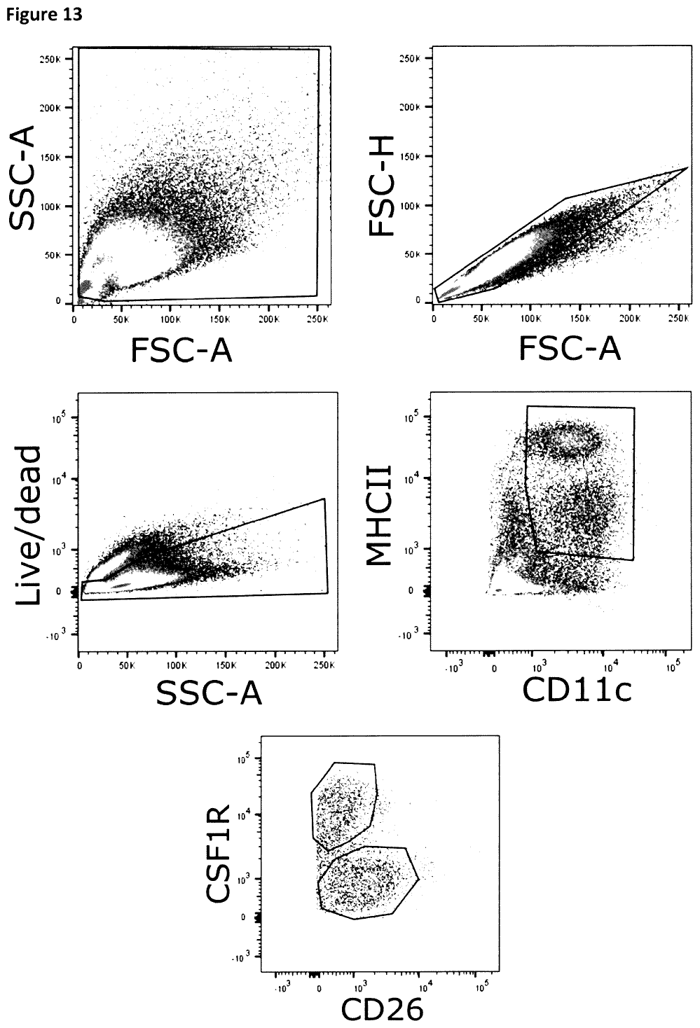

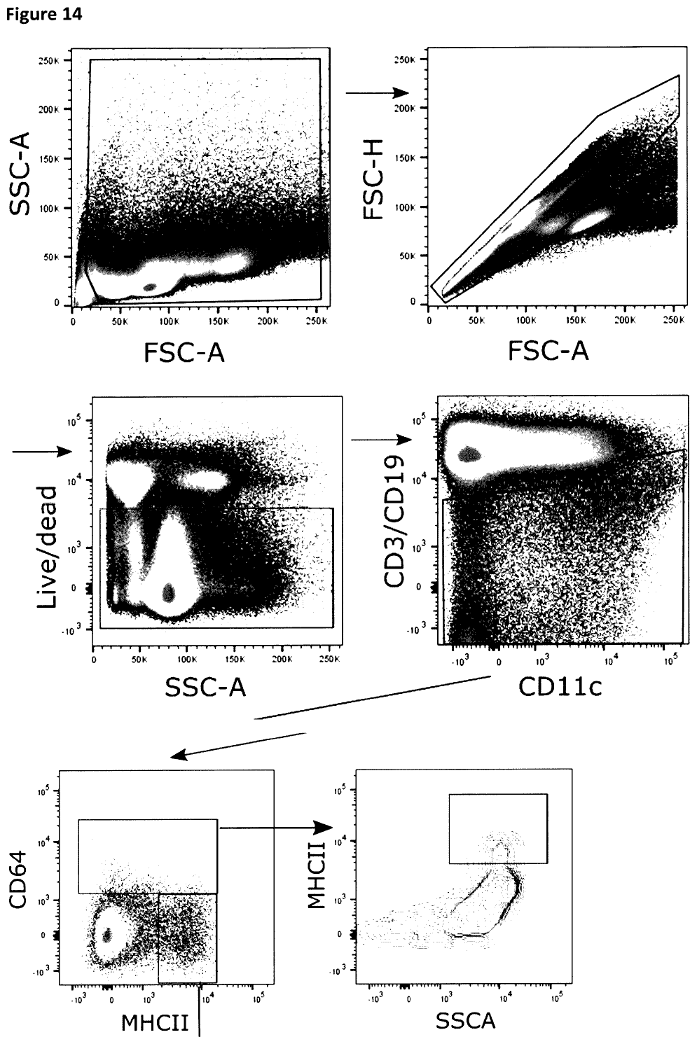

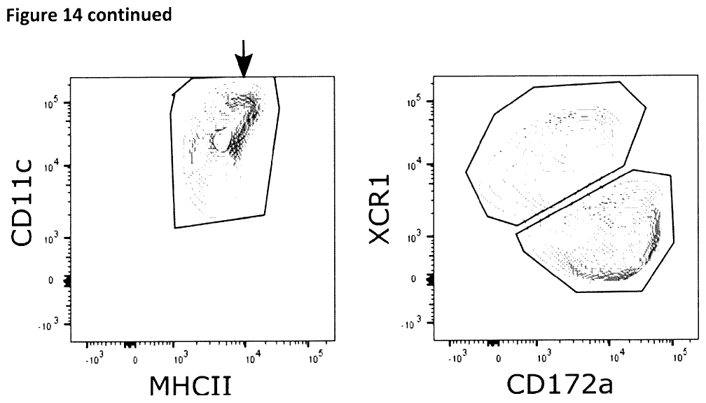

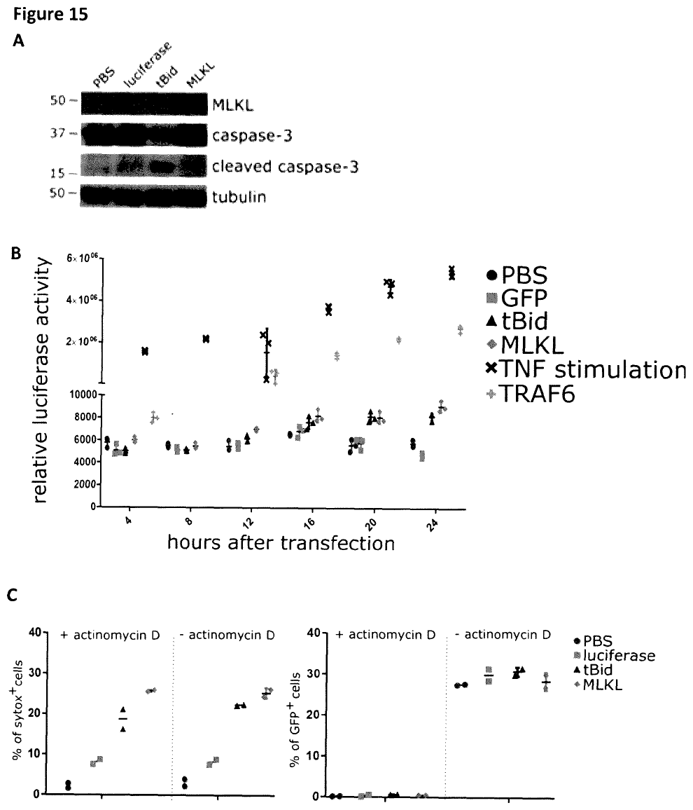

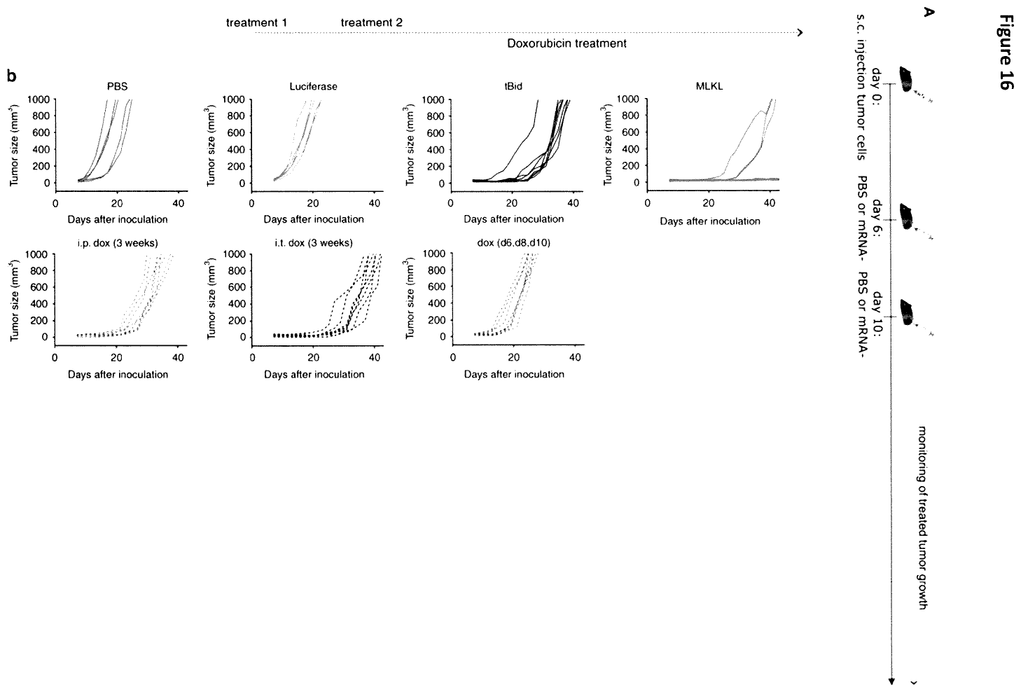

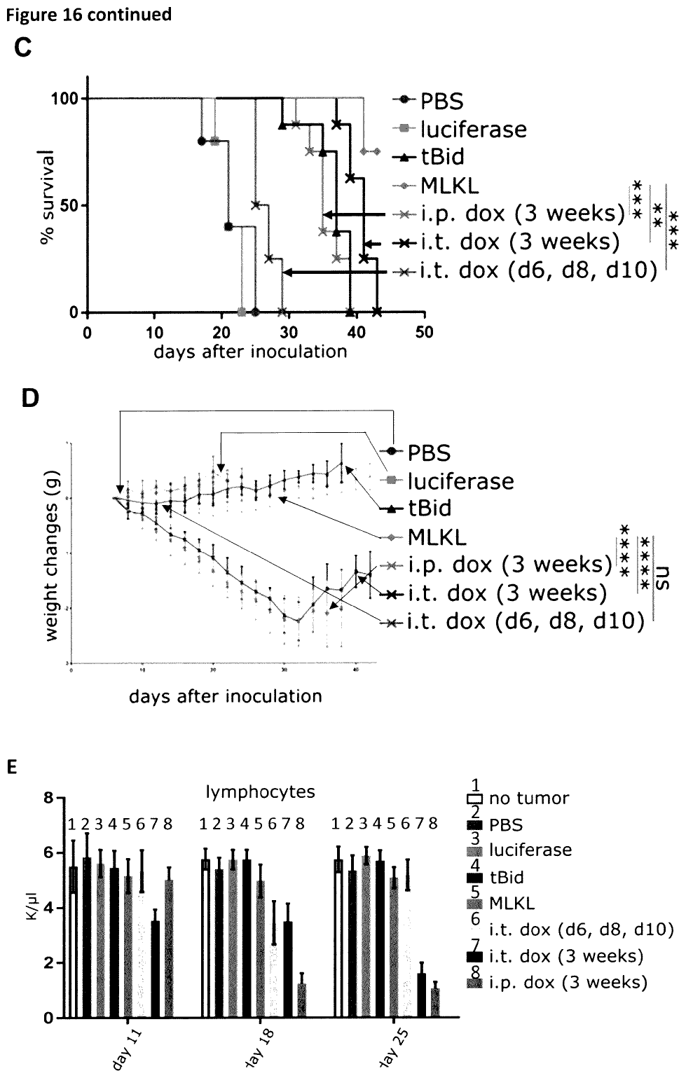

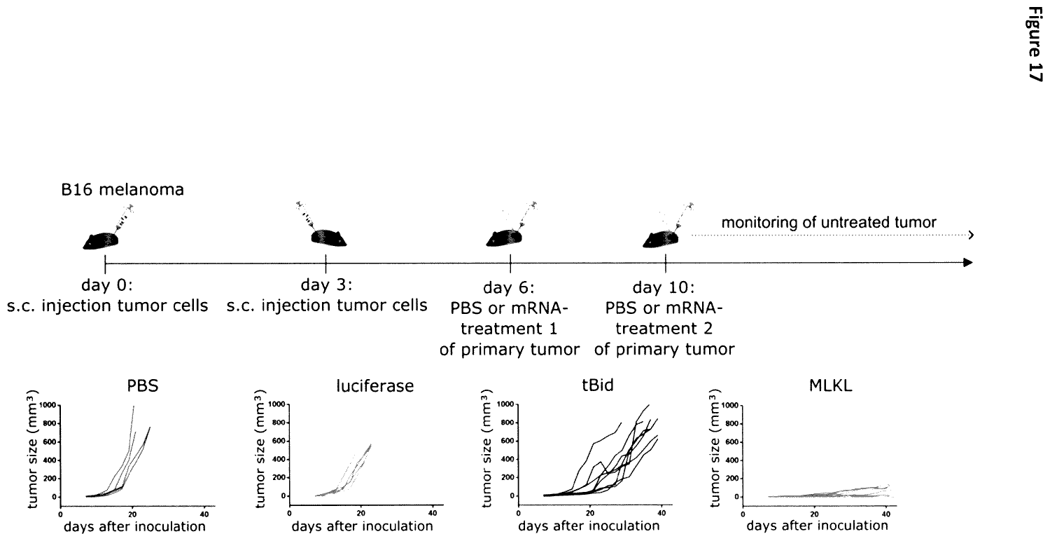

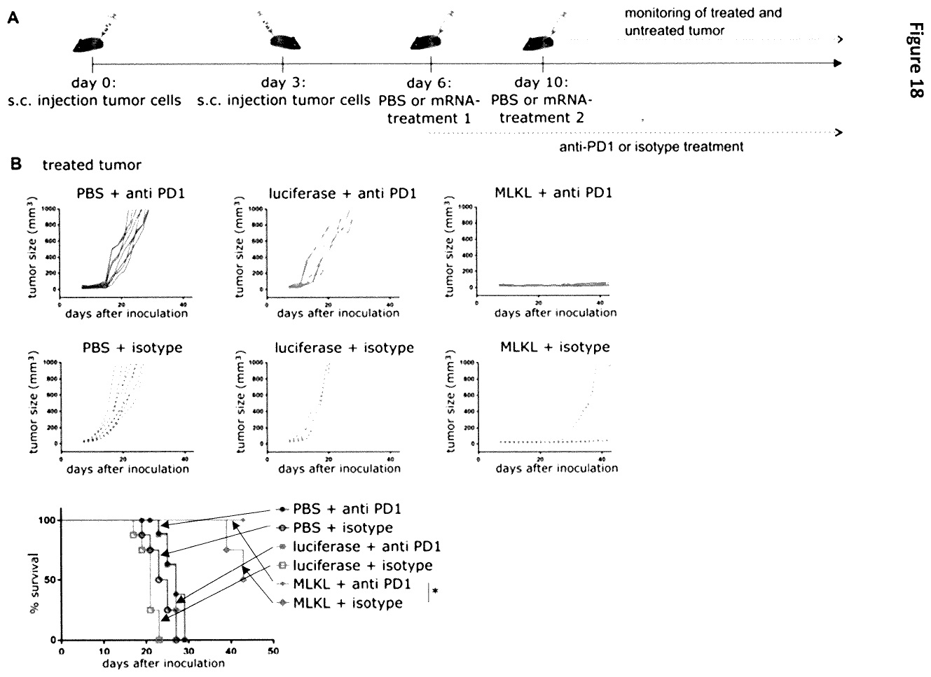

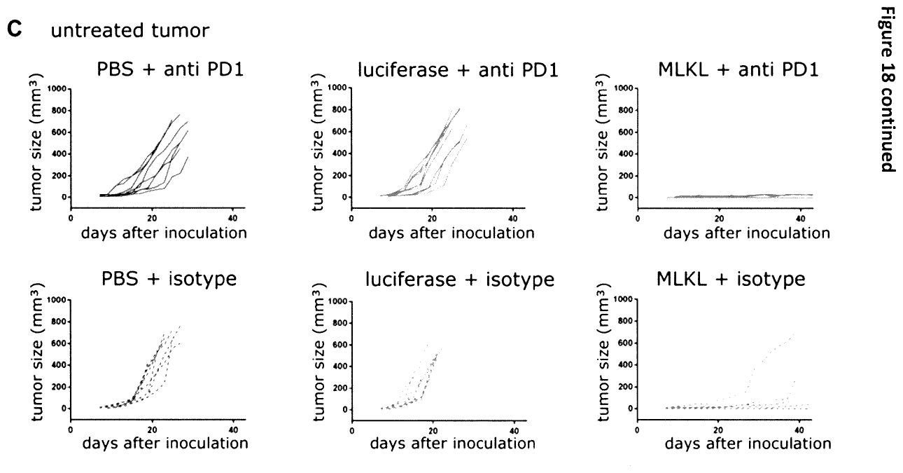

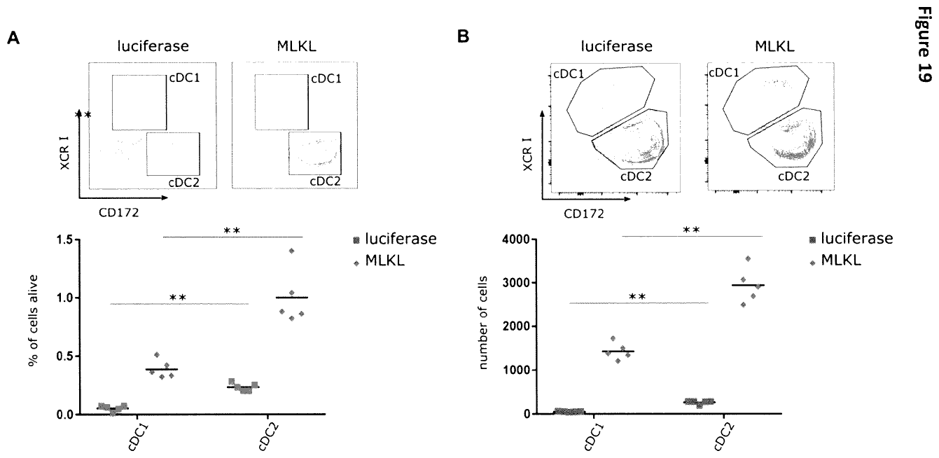

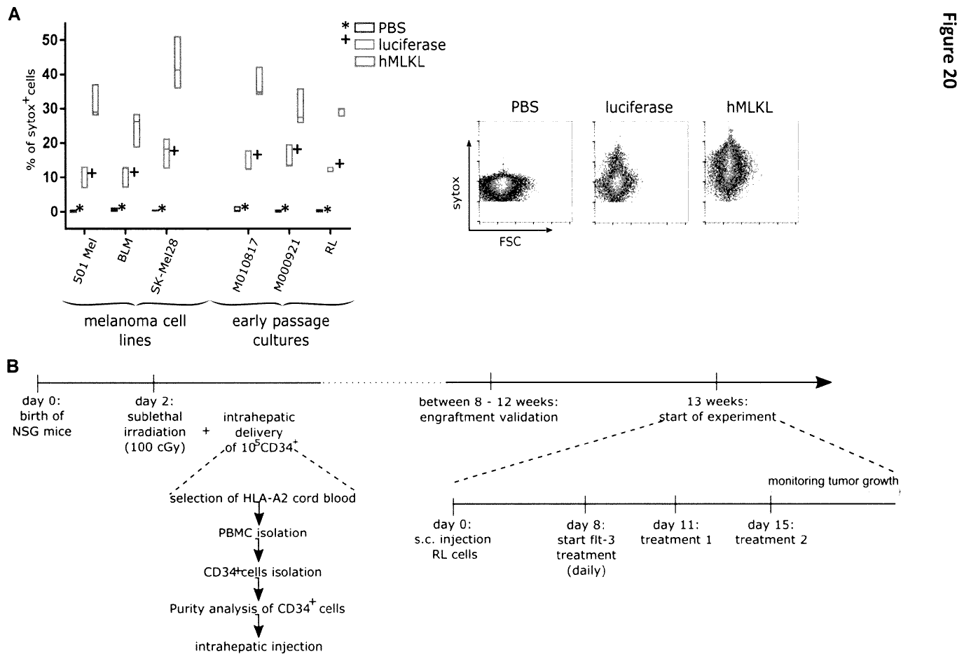

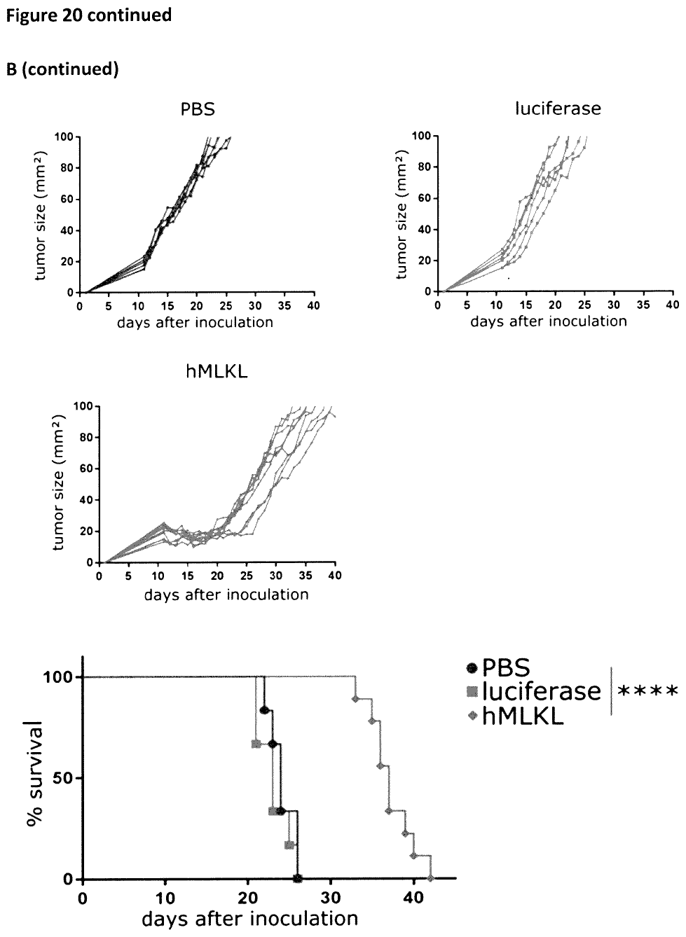

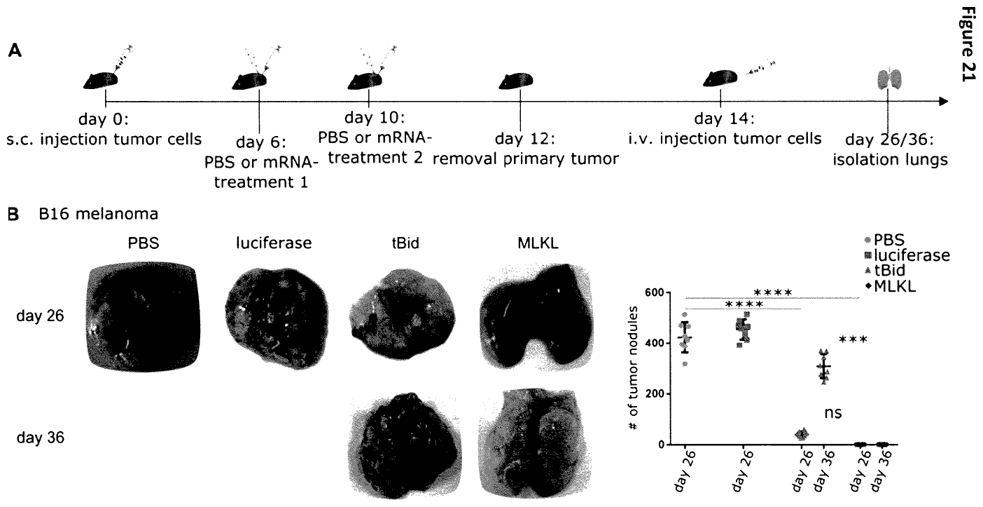

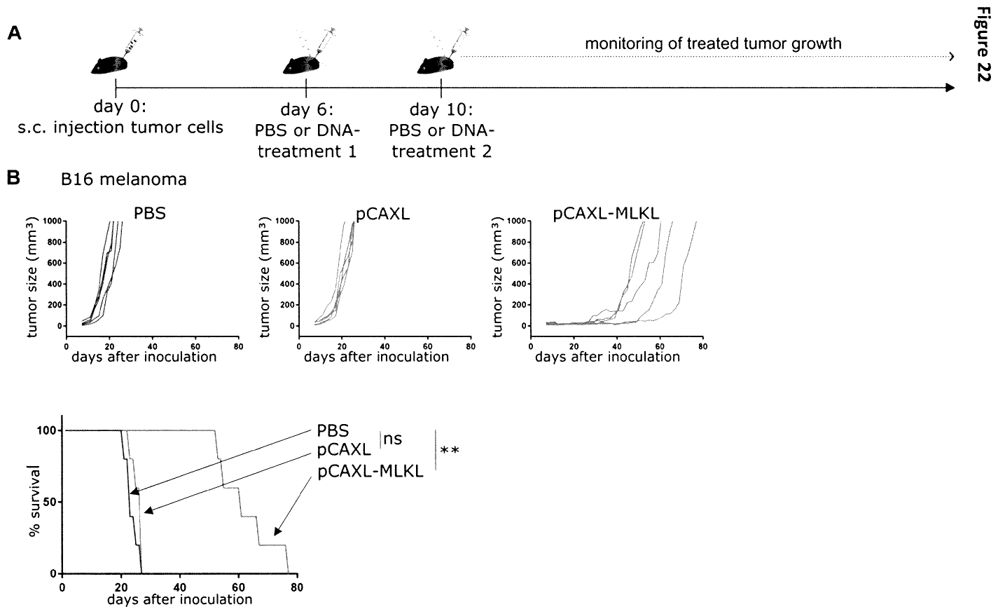

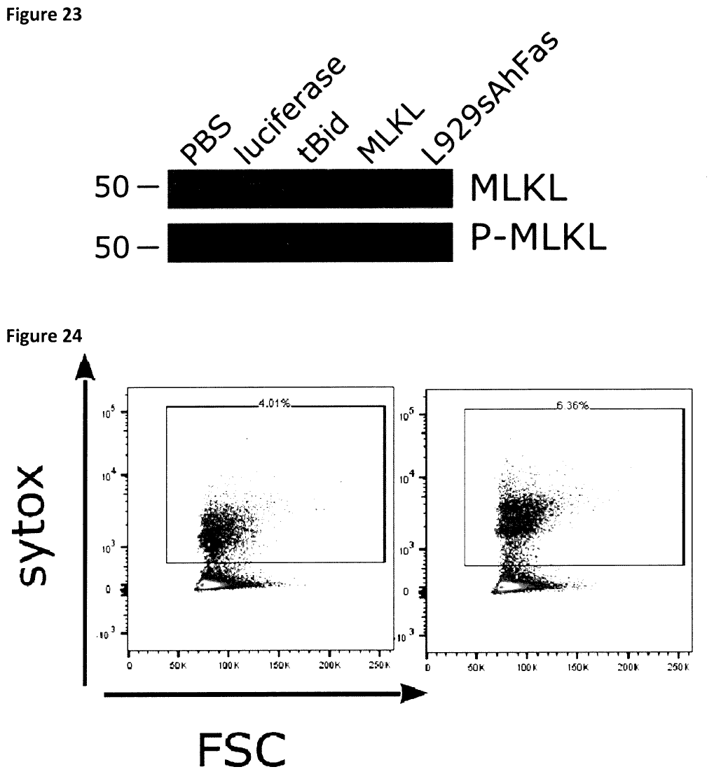

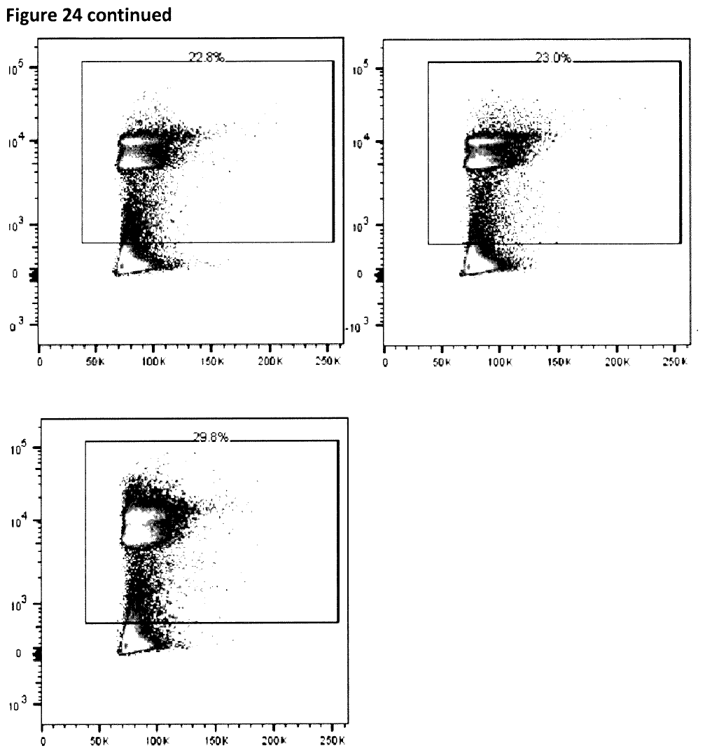

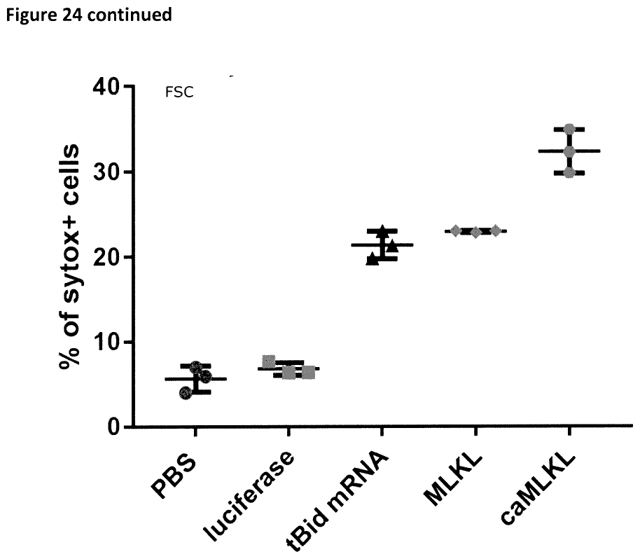

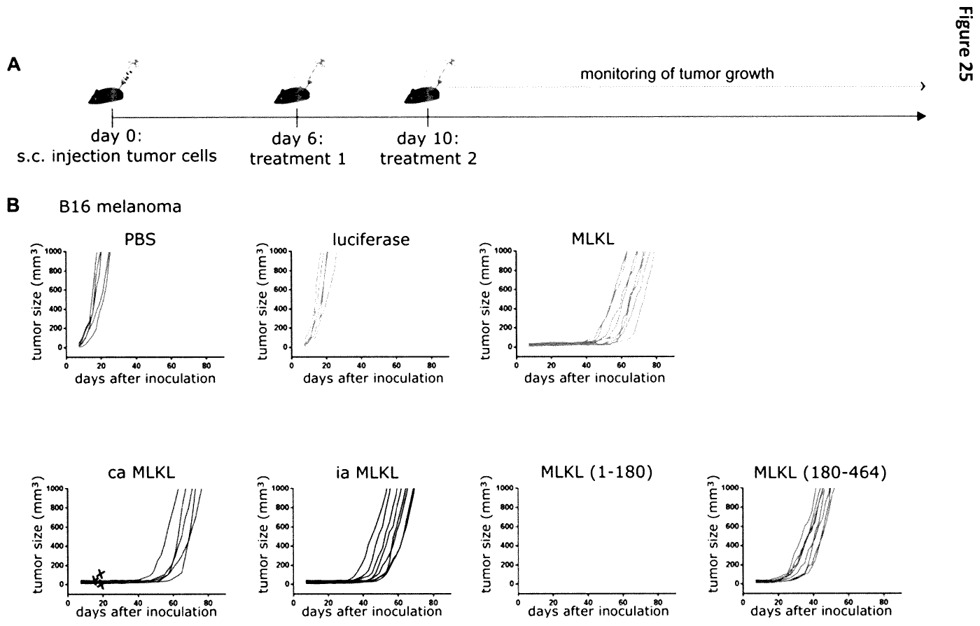

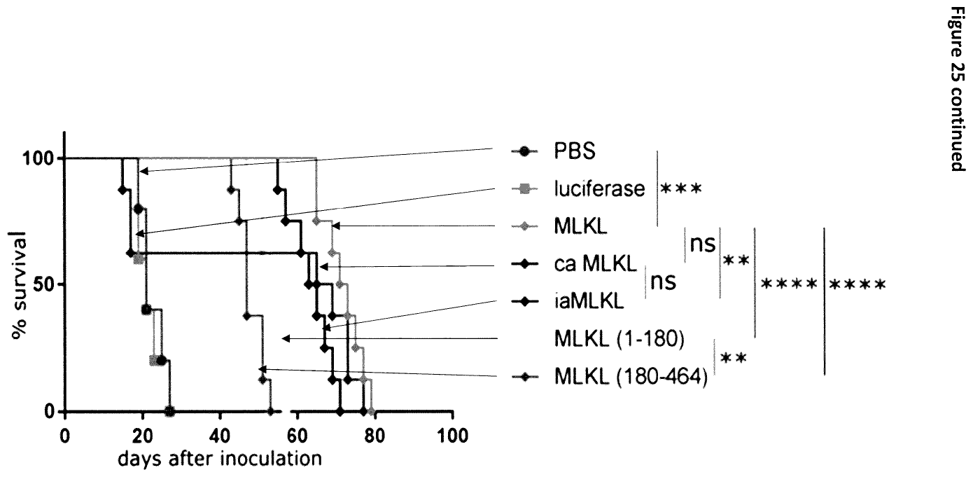

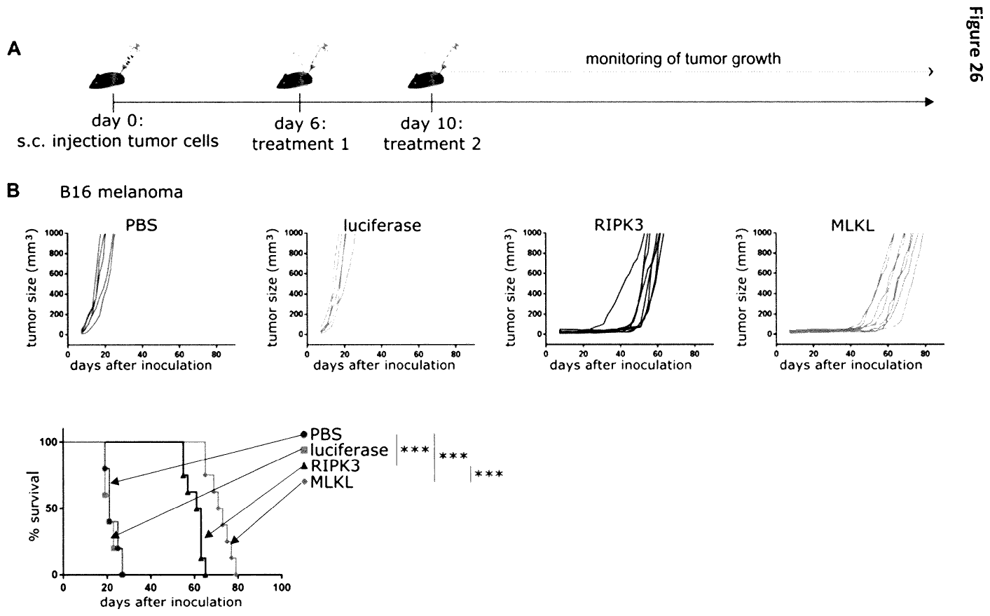

The induction of cell death in the tumor by the injection of mRNA encoding MLKL followed by electroporation could hypothetically promote the induction of anti-tumor T cell responses through the release of tumor antigens alongside danger-associated molecular patterns (DAMPs) that activate the immune system. Such anti-tumor T cell responses can in principle also blunt or prevent non-treated distal tumors and metastases. To address whether local IT treatment of tumors could indeed induce immune related abscopal effects in non-treated tumors, the primary OVA-expressing tumor was surgically removed two days after the second treatment. Another two days after tumor removal, the mice were re-challenged by subcutaneous injection of 500,000 B16 or CT26 cells in the opposite flank (FIGS. 2A and 2B). IT treatment of primary tumors with saline or control mRNA resulted in comparable tumor growth and all control treated mice had to be euthanized by day 44 in the case of the B16 model (FIG. 2A) and before day 42 in the case of the CT26 model (FIG. 2B). tBID mRNA treatment of the primary B16-OVA (FIG. 2A) or CT26-OVA (FIG. 2B) tumor resulted in a modest protection against tumor re-challenge. But clearly, the protection against tumor re-challenge was most pronounced after IT treatment with mRNA encoding MLKL In this case 40% of the B16 inoculated mice were still tumor free by day 86 of the experiment (FIG. 2A) and in the case of CT26 all mice remained tumor free up to 86 days after inoculation of the primary tumor (FIG. 2B).

Systemic immunity was further tested in a second model in which a possible abscopal effect could be evaluated (Singh et al. 2017, Nature Comm 8:1447). Mice were inoculated with B16 cells in either flank but on different days: the tumor in the left flank was implanted three days later than the tumor in the right flank. Only the tumor in the right flank of the animals was subsequently treated, starting day 6 after the first tumor inoculation (and thus 3 days after the injection of the 2^{nd} tumor on the opposite site, which was not treated). The growth of the distant untreated tumor in the left flank was monitored over time (FIG. 17). Also in this set-up a pronounced delay in tumor growth of the untreated tumor was observed in the case of MLKL RNA administration to the treated tumor (FIG. 17).

Encouraged by these results, the protective potential of IT mRNA delivery in a model of metastasis. In this model, as described above, the primary tumor was treated in a prime-boost scheme and removed two days after the second treatment. Another two days later, the mice were challenged by intravenous (i.v.) injection of 200,000 B16-F10 cells (FIG. 3A) or CT26 cells (FIG. 3B). In saline or control mRNA treated mice, this resulted in the rapid development of tumor nodules in the lungs (FIGS. 3A and 3B). In contrast, IT treatment with mRNA coding for MLKL completely protected against tumor nodule formation whereas protection by tBid mRNA was incomplete (FIGS. 3A and 3B). Extended results are shown in FIG. 21.

In summary, these in vivo experiments revealed that IT injection of mRNA encoding MLKL into primary tumors followed by electroporation elicits a strong and systemic anti-tumor immune response. This response is not only able to reduce the primary tumor growth but can also combat secondary tumor growth and on top of that gives protection in a model of tumor metastasis. Moreover, treatment with a necroptosis effector molecule (MLKL) is superior to an apoptosis inducer (tBid) in terms of inducing an anti-tumor immune responses.

A.2.3. Intra-Tumoral Treatment with MLKL mRNA Instigates Cellular Anti-Tumor Responses Directed Against Neo-Epitopes

Next we analyzed the impact of our mRNA based treatment on the magnitude and functionality of the induced T cell responses. First we addressed the effect of IT mRNA treatment on the initial priming of antigen specific CD8⁺ and CD4⁺ T cells because both these cell types are implicated in anti-tumor immunity (Ahrends et al. 2016, Cancer Res 76:2921-2931; Savelveya et al. 2017, Curr Top Microbiol Immunol 405:123-143; Bevan 2004, Nature Rev Immunol 4:595-602; Arens & Schoenberger 2010, Immunol Rev 235:190-205). To this end, B16 melanoma cells transduced with OVA and OVA-specific transgenic CD8⁺ T cells (OT-I) and CD4⁺ T cells (OT-II) were used. Two days before a single IT mRNA treatment, B16-OVA bearing mice received an adoptive transfer of CFSE-labeled OT-I or OT-II cells and the proliferation of these cells was monitored another two days later by flow cytometry of cells isolated from the tumor draining lymph node (see FIG. 11 for the gating strategy). A strongly elevated OT-I and OT-II proliferation of up to 40% and 33%, respectively, was observed in mice that had been treated with mRNA encoding MLKL (FIG. 4A). From this it can be concluded that mRNA encoding MLKL treatment of B16-OVA tumors efficiently primed both CD8⁺ and CD4⁺ T cell.

Next, the impact of the designed treatment on the cytolytic capacity of the treatment induced CD8⁺ T cell response was analyzed by an in vivo killing assay. In brief, B16-OVA bearing mice were IT treated with saline, mRNA encoding luciferase, tBid or MLKL on day 6 and 10 after the tumor cell inoculation. Three days after the second treatment, the mice received a 1:1 ratio of OVA peptide-pulsed CFSE^{hi} splenocytes (target cells) and irrelevant peptide-pulsed CFSE^{low} splenocytes (non-target cells) by i.v. injection (FIG. 4B). Two days later, the mice were sacrificed, the spleens were dissected and the ratio of target cells versus non-target cells was analyzed by flow cytometry to determine the extent of target cell-specific killing (see FIG. 12 for the gating strategy). IT treatment with MLKL mRNA resulted in up to 75% specific killing of target cells compared to 40% of killing in mice that had been treated with mRNA encoding tBid (FIG. 4B). In control treated mice, target cell killing was negligible (FIG. 4B).

Finally, to analyze the effector function of the T cell response, the number of IFN-γ producing OVA-specific CD8⁺ and CD4⁺ T cells was quantified by ELISpot. Splenocytes were used that had been isolated three days after the second IT mRNA treatment. A highly significant increase in the number of MHC class I and II OVA-specific IFN-γ secreting splenocytes derived from mice that had been treated with mRNA encoding MLKL was observed (FIG. 4C). Recently, Kreiter et al. 2015 (Nature 520:692-696) reported on a powerful tool to identify potential neo-epitopes in tumor cells, including the B16 and CT26 cells used here. It was therefore assessed whether IT MLKL mRNA treatment would also result in an immune response against these neo-epitopes, next to the reactivity observed against the exogenous model antigen OVA. In the B16 model, the CD4⁺ T cell response against the B16-M30 class II-restricted neo-epitope was evaluated and, as a control, against the parental non mutated B16-WT30 peptide (FIG. 5A). For the CT26 model, focus was on the CD8⁺ T cell CT26-M26 epitope and the CD4⁺ T cell CT26-M20, CT26-M03, CT26-M37 and CT26-M27 epitopes (FIG. 5B). These epitopes have been described in Kreiter et al. 2015 (Nature 520:692-696) as mutant neo-epitopes that can be used to induce anti-tumor immunity. The results from the ELISpot showed an explicit induction of CD8 and CD4 neo-epitope-specific IFN-γ secreting cells upon IT treatment with mRNA coding for MLKL (FIG. 5).

Taken together, the anti-tumor response that is induced by IT treatment with mRNA coding for MLKL correlates with tumor antigen-specific CD8⁺ and CD4⁺ T cell priming, the induction of a functional cytotoxic T cell response as well as the generation of neo-epitope-specific IFN-γ secreting cells. This shows that the MLKL mRNA-based anti-cancer treatment can circumvent the time consuming identification of patient specific neo-antigens that subsequently still need to be incorporated into a (vectored) vaccination platform before the patient treatment can start. In other words, IT treatment with mRNA encoding MLKL can rapidly induce neo-epitope-specific cellular immune responses, without prior knowledge of the tumor mutanome.

A.2.4. MLKL mRNA Treatment is Associated with cDC1 and cDC2 Activation

The effectiveness of vaccines that intend to induce long-lasting tumor-specific T-cell responses requires the engagement of professional antigen-presenting cells, especially dendritic cells (DCs). Laoui et al. 2016 (Nature Communications 7:13720) showed that distinct subsets of DC populations have different functions in the process that leads to the induction of an anti-tumor T cell response. The canonical view is that monocyte-derived DCs (moDCs) are very efficient in the uptake of tumor antigens but these cells have limited capacity to stimulate T cells (likely due to nitric oxide-mediated immunosuppression). cDC1s on the other hand, efficiently activate CD8⁺ T cells whereas cDC2s are important for the induction of Th17 and Th2 responses (Laoui et al. 2016, Nature Communications 7:13720; Plantinga et al. 2013, Immunity 38:322-335; Persson et al. 2013, Immunity 38:958-969; Gao et al. 2013, Immunity 39:722-732; Schlitzer et al. 2013, Immunity 38:970-983).

Apoptosis and necroptosis have been exploited to stimulate adaptive immune responses against co-delivered vaccine antigens (Aaes et al. 2016, Cell Rep 15:274-287; Sasaki et al. 2001, Nature Biotechnol 19:543-547). In line with this, it was found that in vitro co-culture of tBid and MLKL mRNA transfected B16 melanoma cells with bone marrow derived dendritic cells and macrophages resulted in upregulation of the activation markers CD40, CD80 and CD86 in the antigen-presenting cell population (FIG. 13).

Next, the in vivo influx of different DC subtypes and their activation state in the tumor draining lymph node after control, tBid or MLKL mRNA treatment was analyzed. Two days after the second treatment of B16-OVA tumor-bearing mice, the draining lymph nodes were dissected and the influx of different DC subtypes was examined by flow cytometry (FIG. 6; see FIG. 14 for the gating strategy). A strong influx of type 1 and 2 cDCs as well as moDCs was apparent in the draining lymph nodes of mice that had been IT treated with mRNA coding for MLKL. This influx was much more modest in the tBid mRNA treated group and negligible in the mock and negative control treated animals (FIG. 6).

Type I IFN-mediated activation of the Batf3-dependent CD103⁺ DC subset is critically required for the spontaneous induction of antitumor T cell responses and for the therapeutic benefit of intratumor treatment with TLR and STING agonists, and oncolytic viruses (Curran et al. 2016, Cell Rep 15:2357-2366; Foote et al. 2017, Cancer Immunol Res 5:468-479; Heinrich et al. 2017, Oncotargets Ther 10:2389-2401; Kim et al. 2015, Viruses 7:6506-6525). To gain more insight in the immune pathways responsible for the MLKL-mRNA mediated antitumor responses, we probed the influx of DCs in the tumor bed and in the tumor draining lymph node by flow cytometry on day 1 and 2, respectively, after two intratumor treatments with mRNA encoding MLKL (FIG. 19A; FIG. 14 for the gating strategy). Batf3-dependent DCs (conventional type 1 DCs or cDC1) and IRF4-dependent DCs (cDC2) represent the two major classes of DCs and can be discriminated by their distinct expression of XCR1 (cDC1) versus CD172α (cDC2). Compared to mock treated mice, a strong influx of cDC1 and cDC2 DCs was apparent in the tumor bed and its draining lymph nodes in mice that had been treated intratumorally with mRNA coding for MLKL (FIGS. 19A and 19B).

To study the contribution of DC and T cell migration between the tumor site and the tumor-draining lymph node for the induction of tumor antigen-specific T cell priming by MLKL mRNA treatment, a proliferation assay as described in FIG. 4A was performed in wild type and CC-chemokine receptor 7 (CCR7)-deficient mice. Mice that are deficient in this chemokine receptor show impaired homing of T cells and DCs from the tissue to the draining lymph nodes (Scimone et al. 2006, Proc Natl Acad Sci USA 103:7006-7011; Sallusto et al. 2004, Ann Rev Immunol 22:745-763; Braun 2011, Nature 472:423-424). IT treatment with mRNA encoding MLKL of B16-OVA bearing CCR7-deficient mice was not associated with OT-I proliferation (FIG. 7A) which was in stark contrast to the OT-I proliferation observed in WT mice.

Next, the possible role of CD8α⁺ DCs in the induction of cytolytic CD8⁺ T cell responses after IT mRNA treatment was studied. To this end a killing assay in Batf3-deficient mice, which lack CD8α⁺ DCs in lymphoid tissues (Hildner et al. 2008, Science 322:1097-1100) was performed. In contrast to wt mice, B16-OVA inoculated Batf3 knockout mice did not mount a cytotoxic T cell response upon IT treatment with MLKL or tBid mRNA (FIG. 7B). These two experiments suggest that intact lymphocyte homing and CD8α⁺ DCs are required for the induction of a tumor antigen-specific CD8⁺ T cell response after IT treatment with mRNA encoding MLKL and tBid.

mRNA vaccines, especially when complexed with lipids, can elicit a strong induction of type I interferons (IFNs), that are known as potent inflammatory cytokines that impact T cell differentiation and survival (Pollard et al. 2013, Mol Ther 21:251-259). Recent reports have attributed opposing roles, i.e. profoundly stimulatory to strongly inhibitory, for type I IFN signaling in modulating CD8⁺ T cell immunity induced by mRNA vaccines. The mechanisms behind this duality remains unclear (Broos et al. 2016, Mol Ther Nucl Acids 5:e326; Kranz et al. 2016, Nature 534:396-401; De Beuckelaer et al. 2016, Mol Ther 24:2012-2020; De Beuckelaer et al. 2017, Trends Mol Med 23:216-226). To clarify the possible involvement of type I IFN signaling in our mRNA treatment strategy, an in vivo killing in IFNARI^{-/-} mice (FIG. 7C) was performed. IT mRNA treatment of IFNARI^{-/-} did not result in detectable cytolytic activity in any of the four settings, suggesting that type I IFNs are necessary for the induction of cytolytic CD8⁺ T cell responses upon IT treatment with mRNA encoding MLKL (FIG. 7C).

The data above show that the anti-tumor response induced by IT treatment with mRNA coding for MLKL correlates with the induction of tumor antigen-specific CD8⁺ and CD4⁺ T cell priming and establishment of tumor epitope-specific effector CD8⁺ and CD4⁺ T cells (FIGS. 4 and 5). The contribution of CD8⁺ and CD4⁺ T cells to the protection against primary tumor growth by the mRNA treatment was determined next. To this end, CD4⁺ or CD8⁺ T cells were depleted by antibody treatment as described in Van Lint et al. (unpublished). It was found that deletion of CD8⁺ T cells and to a lesser extent of CD4⁺ T cells abolished the anti-tumor effect evoked by the IT treatment with mRNA encoding MLKL (FIGS. 7D and 7E). These results indicate that both CD8⁺ and CD4⁺ T cells are essential for the therapeutic anti-tumor effect of MLKL mRNA.

A.2.5. Combining MLKL-mRNA Treatment with PD1 Blockage Improves the Anti-Tumor Effect

The anti-tumor activity of MLKL-mRNA treatment might be further improved upon combination with cancer treatment options that are already clinically established such as checkpoint blockade approaches. Once inside the tumor bed, T cells primed by intratumor MLKL-mRNA treatment might be silenced by multiple immune suppressive mechanisms used by tumors to evade elimination (Chen & Mellman 2013, Immunity 39:1-10). Checkpoint inhibitors such as anti-CTLA4, -PD-1 and -PD-L1, IDO inhibitors or Treg depletion strategies primarily act by taking away these breaks yet are poorly effective in patients with tumors with a low number of tumor-infiltrating T cells (Tumeh et al. 2014, Nature 515:568-571). Since the MLKL-based mRNA therapy reported here induces robust infiltration of APCs into the tumor, it is possible that a combination therapy with a checkpoint inhibitor could further improve the curative potential of intratumor delivery of MLKL-mRNA. B16 tumors were implanted s.c. in the right flank of the mice and three days later in the left flank of the mice, followed by i.t. treatment with MLKL in combination with i.p. administration of anti-PD-1 (FIG. 18). This combination therapy was significantly more effective at stalling the growth of the primary treated tumor and the growth of the distant untreated tumor than the MLKL-treatment on its own (FIG. 18).

A.2.6. Intra-Tumoral Delivery of MLKL Encoding Plasmid DNA Stalls Primary Tumor Growth

Mouse MLKL cDNA was cloned in the pCAXL plasmid under the transcriptional control of the chicken β-actin/rabbit β-globin hybrid promoter and human cytomegalovirus immediate early promoter enhancer. The resulting plasmid was named pCAXL-MLKL, amplified in E. coli DH5α and purified using an endotoxin free plasmid preparation kit (Qiagen).

In total 5.10⁵ B16 (OVA) cells in 100 μl of Hank's Balanced Salt Solution (HBSS; Gibco) were injected subcutaneously into the right flank of C57BL/6 mice. On day six and ten after inoculation of the tumor cells, 100 μg of DNA dissolved in 10 μl of HBSS was injected in the tumor using a U-100 insulin needle (BD Biosciences, San Diego, Calif., USA). Next a conductive gel (EKO-GEL, ultrasound transmission gel, Egna, Italy) was applied at the tumor site to ensure electrical contact of the electrodes with the skin and electroporation was performed. Eight pulses of 20 ms and 100 V/cm were delivered through spaced plate electrodes by a ECM® 830 Electroporation System (BTX® Harvard apparatus). The tumor size was measured every two days with an electronic digital caliper. The tumor volume was calculated as the length×width×height (in mm³). The mice were humanely euthanized when the volume of the tumor had reached 1000 mm³.

Results of an initial experiment are depicted in FIG. 22 and basically mirror the results obtained with MLKL-encoding RNA regarding stalling of primary tumor growth (A.2.2). These data provide initial support for application of plasmid-encoded MLKL as a means for protecting against tumor rechallenge and metastasis.

A.2.7. MLKL Protein Expressed from MLKL mRNA is not Phosphorylated

The phosphorylation status of MLKL in mRNA transfected B16 melanoma cells was checked. As a positive control for necroptosis induction, lysates of L929sAhFas cells that had been stimulated with TNF for 8 hours were included (Vercammen et al. 1998, J. Exp. Med. 188, 919-930; Krysko et al. 2003, J Morphol 258:336-345). MLKL was detectable in lysates of B16 cells that had been transfected with MLKL mRNA and of L929sAhFas. Phosphorylated MLKL, however, was only detectable in the TNF stimulated L929sAhFas cell lysates (FIG. 23).

A.2.8. Effect of Constitutive MLKL Mutants

B16 melanoma cells were transfected with mRNA coding for luciferase, tBid, MLKL and a constitutively active mutant of MLKL (MLKLS345D, abbreviated caMLKL; Murphy et al. 2013, Immunity 39:443-453). Twenty four hours after transfection, cell viability was monitored by flow cytometry and the percentage of sytox blue positive cells was determined. Transfection with caMLKL mRNA was associated with an increased percentage of cells that became sytox positive compared to tBid and MLKL mRNA transfected cells (approximately 30% compared with approximately 20% for tBid or MLKL mRNA)(FIG. 24).

The in vitro effect of other MLKL mutants as described hereinabove is tested.

The in vivo anti-tumor response of caMLKL variant mRNA and of other MLKL mutants as described hereinabove is tested.

It has been reported that an MLKL fragment comprising amino acids 1-180 (4HD domain) can induce cell death in mouse dermal fibroblasts independent of caspase or RIPK1 activity and independent of the presence of RIPK3 (Hildebrand et al. 2014, Proc Nat Acad Sci USA 111:15072-15077. Therefore, an experiment was set up wherein it was tested if mRNA encoding an MLKL1-180 or MLKL 180-464 (encompassing the pseudokinase domain) could delay B16-Ova tumor growth as efficiently as a full length MLKL construct. In addition, it was evaluated whether a non-phosphorylatable mutant (“iaMLKL”: full-length MLKL harboring the mutation of Ser345 to Ala, S345A) as well as a “constitutively active” MLKL variant (“caMLKL”: full-length MLKL harboring the mutation Ser345 to Asp, S345D) would be able to retard B16-Ova tumor outgrowth. It was found that wtMLKL performed the best followed by caMLKL although in this group 3 out of 8 mice had to be euthanized because of the development of severe lesions at the tumor site by day 20. iaMLKL performed slightly less well than wtMLKL mRNA and the two fragments performed intermediate between the control mRNA groups and wt MLKL. Results are shown in FIG. 25.

A.2.9. Anti-Tumor Effect of Intratumoral Delivery of MLKL Nucleic Acid is More Potent than of Intratumoral Delivery or RIPK3 Nucleic Acid

RIPK3 is the upstream kinase of MLKL (Sun et al. 2012, Cell 148:213-227). An experiment was set up in which the effect of treatment with equal amounts of intratumor MLKL-encoding mRNA and RIPK3-encoding mRNA on tumor growth were compared. The results are shown in FIG. 26 and indicate that MLKL mRNA delayed B16-Ova tumor growth significantly better than RIPK3 mRNA.

A.2.10. Transduced MLKL Protein Induces Necroptotic-Like Cell Death in Tumor Cells In Vitro

On day 1, 30×10\hat{ }3 B16 cells are seeded in 96-well plate. On day 2, 8×10\hat{ }7 AuNPs/ml (gold-coated nanoparticles, 70 nm) are added to the cells. During a 30 minutes incubation, the gold nanoparticles are adsorbed to the cell membrane. Next, the MLKL-protein is added to the extracellular medium and the photoporation treatment is performed. A homemade setup including an optical system and electric timing system is used for photoporation. A pulsed laser illuminate the AuNPs. In this way vapour nanobubbles create transient openings in the cell membrane and the MLKL-protein can passively diffuse through the pore. Cell death is subsequently analyzed via sytox blue staining and flow cytometry. At first instance, wild-type MLKL protein is applied in this assay and results are compared with those obtained with transfected MLKL protein-encoding mRNA (as described hereinabove).

### B. Human Tumor Cell Lines

B.1. Evaluation of Cell Death Evoked by MLKL or tBid mRNA in Human Cancer Cells In Vitro

To show the potential of the described IT mRNA treatment strategy in human cancer cells, the degree and type of cell death following transfection with mRNA coding for human tBid and MLKL is analyzed in human melanoma cell lines (501Mel, SKMel28 and BLM) and also in primary human tumor tissue-derived cells. The cells are mock transfected or transfected with mRNA coding for luciferase, tBid or MLKL and subsequently stained with SYTOX blue to determine membrane permeability and annexin-V for phosphatidylserine exposure at the membrane. The percentages of annexin⁺/SYTOX blue⁺ cells (left) and annexin⁻/SYTOX blue⁺ cells (right) of the total single cell population are determined. The type of cell death, apoptotic or necroptotic, is determined.

In a first set of experiments, the sensitivity of different human melanoma cell lines and early passage human tumor cells to cell death induced after transfection of mRNA encoding hMLKL was evaluated. Flow cytometry analyses of mRNA-transfected human melanoma cell lines, early passage melanoma cells and RL human B lymphoma cells showed that, unlike mock transfection, Fluc-mRNA (luciferase) and more extensively hMLKL-mRNA transfection resulted in cell death (FIG. 20A).

B.1. Evaluation of Protective Immune Response Against Human Cancer Cells In Vivo

Mice with a fully humanized immune system are inoculated with human melanoma cancer cells. The primary tumor is treated by intra-tumoral administration of human MLKL mRNA. The treated primary tumor is surgically removed and the mice are subsequently challenged by inoculation with untreated human melanoma cancer cells at a site remote from the primary tumor site. The abscopal effect of the treatment of the primary tumor with human MLKL mRNA is determined.

To assess the therapeutic potential of intratumor hMLKL-mRNA treatment of a human tumor in vivo, mice with a humanized adaptive immune system were used. Irradiated newborn NOD-SCID-gamma (NSG) mice that had received an intrahepatic injection of human CD34⁺ stem cells, were inoculated s.c. with 2.5×10⁶ human RL follicular lymphoma cells. At day 11 and 15, when a palpable tumor could be observed, the tumors were treated with saline or with mRNA encoding Fluc or hMLKL (FIG. 20B). Intratumor administration of saline or mRNA encoding Fluc (luciferase) resulted in comparable tumor growth. However, a striking antitumor effect following intratumor treatment with hMLKL-mRNA was observed, which significantly delayed tumor growth and increased the median survival time of the mice (FIG. 20B). These results point to hMLKL-mRNA-based antitumor treatment as a sound candidate for testing in a clinical setting.

### C. Conclusion

Most cancer cells express mutant proteins that can be recognized by the adaptive immune system. Therapeutic cancer vaccine approaches aim at inducing a protective T cell response against these mutant proteins, also named neo-antigens. However, the composition of these vaccines requires detailed knowledge of the neo-antigens of an individual patient's tumor before a personalized treatment can be started. We developed a generic mRNA-based therapy to elicit a highly protective CD4⁺ and CD8⁺ T cell response directed against tumor specific neo-antigen epitopes without prior knowledge of the tumor mutatome. This therapy is based on the controlled delivery of mRNA that encodes the mixed lineage kinase domain-like (MLKL) protein. Transient in vitro and in vivo expression of MLKL in tumor cells resulted in necroptotic cell death. Furthermore, intra-tumor delivery of MLKL-mRNA stalled the growth of primary tumors and protected against distal and metastatic tumors in syngeneic mouse melanoma and colon carcinoma tumor models. The MLKL-mRNA treatment induced infiltration of moDCs, cDC1s and cDC2s and the anti-tumor immunity was dependent on CD103⁺/CD8α⁺ DCs.

To address the question if an intratumor MLKL-mRNA treatment method holds promise for clinical application, experiments were performed in mice with a grafted human immune system that were subsequently inoculated with HLA-matched human lymphoma-derived cancer cells. In these mice, hMLKL-mRNA treatment strongly suppressed tumor growth, suggesting that this approach could be effective in the clinic.

Further, it was shown that MLKL encoded by DNA (exemplified by plasmid DNA) recapitulates the in vivo effects of MLKL mRNA; this renders extrapolation to recapitulation of the in vitro and in vivo effects by means of MLKL protein administration plausible.

Finally, it was shown that combining treatment with MLKL mRNA with an immune checkpoint inhibitor further improves the anti-tumorigenic effect. The anti-tumorigenic effect of MLKL mRNA was superior to that of doxorubicin, with the further advantage of not displaying the negative side effects of doxorubicin.

These combined results strongly suggest that MLKL-based tumor treatment can be exploited as an immunotherapeutic strategy in human cancers.

It has been reported that chemically induced RIPK3 dimerisation can trigger immunogenic cell death and anti-tumor immunity, by a process that requires the production of inflammatory cytokines by the dying cells and NF-κB-dependent cytokine expression (Yatim et al. 2015, Science 350: 328-334). However, no evidence was found that NF-κB was activated following MLKL mRNA transfection.

Several lines of evidence gathered hereinabove indicate that the therapeutic effect of MLKL nucleic acid (and plausibly MLKL protein) administered to a tumor is independent of RIPK3.

It is furthermore surprising that transfected mRNA encoding wild type MLKL could kill cells because it has been reported that induced expression of wild type MLKL in mouse dermal fibroblasts failed to do so (Murphy et al. 2013, Immunity 39:443-453). Not only wild-type MLKL, but also several fragments and variants of MLKL, were shown hereinabove to have a therapeutic effect.

In the fight against cancer, active immunization strategies are being pursued to evoke T cells recognizing an individual patient's tumor neo-epitopes (e.g. Sahin et al. 2017, Nature 547:222-226). A limitation of such a strategy is the critical dependency on the reliability of the algorithms used to predict the immunogenicity of mutated peptide sequences. In addition, neo-epitopes are highly specific for a given tumor and patient, which implies that for each patient a personalized new vector or delivery system has to be generated to elicit neo-epitope-specific responses (Schumacher et al. 2015, Science 348:69-74; Vormehr et al. 2016, Curr Opin Immunol 39:14-22). The MLKL-mRNA or plasmid DNA-based therapy described here, resulted in the clear induction of tumor antigen-specific CD4⁺ and CD8⁺ T cell responses without a necessity for tumor sequencing, epitope prediction and production of a personalized vaccine vector.

1.-24. (canceled)

25. A method of immunotherapeutic treatment, immunotherapeutic suppression, or immunotherapeutic inhibition of a tumor, cancer, or neoplasm in a mammal harboring the tumor, cancer, or neoplasm, the method comprising administering an effective amount of a nucleic acid encoding a mixed-lineage kinase domain-like (MLKL) protein or an isolated MLKL protein to the mammal.

26. The method of claim 25, wherein the tumor, cancer or neoplasm is deficient in receptor-interacting serine/threonine protein kinase 3 (RIPK3).

27. The method of claim 25, wherein the method is combined with a further therapy against the tumor, cancer or neoplasm.

28. The method of claim 27, wherein the further therapy is surgery, radiation, chemotherapy, immune checkpoint or other immune stimulating therapy, neo-antigen or neo-epitope vaccination, cancer vaccine administration, oncolytic virus therapy, antibody therapy, or other nucleic acid therapy targeting or treating the tumor, cancer or neoplasm.

29. The method of claim 25, wherein the nucleic acid encodes a full-length wild-type MLKL protein, a full-length MLKL protein comprising an amino acid substitution, a fragment of wild-type MLKL protein, or a fragment of a MLKL protein comprising an amino acid substitution.

30. The method of claim 25, wherein the nucleic acid is a hypo-inflammatory nucleic acid.

31. The method of claim 25, wherein the nucleic acid is DNA or RNA.

32. The method of claim 31, wherein the DNA is naked DNA, plasmid DNA, DNA included in a viral vector, or complexed DNA.

33. The method of claim 31, wherein the RNA is naked RNA, RNA included in a viral vector, mRNA, or complexed (m)RNA.

34. The method of claim 33, wherein the mRNA comprises a 5′ cap and/or a 3′ poly(A)tail and/or a 5′ untranslated region and/or a 3′ untranslated region.

35. The method of claim 25, wherein the nucleic acid is administered by intra-tumor, intra-cancer or intra-neoplasm delivery, or wherein the nucleic acid is administered remotely from the tumor, cancer or neoplasm.

36. The method of claim 25, wherein expression of the MLKL protein in the tumor, cancer or neoplasm is transient or inducible.

37. A method of inducing or enhancing necroptotic-like death of or an immune response to a tumor, a cancer, or neoplasm cells in a mammal harboring the tumor, cancer, or neoplasm cells, the method comprising administering an effective amount of a nucleic acid encoding a mixed-lineage kinase domain-like (MLKL) protein or an isolated MLKL protein to the mammal.

38. The method of claim 37, wherein the immune response is an adaptive immune response or a cellular immune response.

39. A method of treating, suppressing, or inhibiting a secondary tumor, cancer, or neoplasm growth in a mammal harboring the secondary tumor, cancer, or neoplasm, the method comprising administering an effective amount of a nucleic acid encoding a mixed-lineage kinase domain-like (MLKL) protein or an isolated MLKL protein.

40. A medicament or a pharmaceutical composition comprising:
(a) an isolated full-length wild-type mixed-lineage kinase domain-like (MLKL) protein, an isolated full-length MLKL protein comprising an amino acid substitution, an isolated fragment of wild-type MLKL protein, or an isolated fragment of a MLKL protein comprising an amino acid substitution; or
(b) a nucleic acid encoding a full-length wild-type MLKL protein, a full-length MLKL protein comprising an amino acid substitution, a fragment of wild-type MLKL protein, or a fragment of a MLKL protein comprising an amino acid substitution.
